(19) United States
(12) Patent Application Publication
(10) Pub. No.: US 2019/0135793 A1
(43) Pub. Date: May 9, 2019

(54) HETEROCYCLIC INHIBITORS OF KDM5 FOR THE TREATMENT OF DISEASE

(71) Applicant: Board of Regents, The University of Texas System, Austin, TX (US)

(72) Inventors: Alessia PETROCCHI, Houston, TX (US); Maria Emilia DI FRANCESCO, Houston, TX (US); Philip JONES, Houston, TX (US); Richard Thomas LEWIS, Missouri City, TX (US); Naphtali REYNA, Arlington, TX (US); Matthew HAMILTON, Missouri City, TX (US); Michelle HAN, Houston, TX (US)

(21) Appl. No.: 15/982,740

(22) Filed: May 17, 2018

Related U.S. Application Data
(60) Provisional application No. 62/508,137, filed on May 18, 2017.

(51) Int. Cl. C07D 403/14; A61K 45/06; A61K 31/5377; A61K 31/506; C07D 417/14; C07D 417/04; C07D 413/14; C07D 405/14; C07D 401/14; C07D 403/04; C07D 401/04

(52) U.S. Cl. CPC C07D 403/14; A61K 45/06; A61K 31/5377; A61K 31/506; C07D 417/14; C07D 401/04; C07D 413/14; C07D 405/14; C07D 401/14; C07D 403/04; C07D 417/04

(57) ABSTRACT

The present invention relates to compounds and methods useful as inhibitors of KDM5 for the treatment or prevention of cancer.

This application claims the benefit of priority of U.S. Provisional Application No. 62/508,137, filed May 18, 2017, the disclosure of which is hereby incorporated by reference as if written herein in its entirety.

Disclosed herein are new heterocyclic compounds and compositions and their application as pharmaceuticals for the treatment of disease. Methods of inhibition of KDM5/JARID1 activity in a human or animal subject are also provided for the treatment diseases such as cancer.

There is growing evidence that the Jumonji family of demethylases are deregulated across a number of disease, notably cancer, and therefore inhibition of their function will have a positive impact on human health. Particularly their utility in the treatment of cancers or other indications where regulation of epigenetic marks plays a role in the disease context. (Hancock et al, Epigenomics (2015), 7(5), 791-811, Zheng et al Medicinal Research Reviews (2015), 35(5), 1032-1071, Chin et al Expert Opinion on Therapeutic Patents (2015), 25(2), 135-144.)

Inhibitors of the histone demethylase class of epigenetic enzymes present a novel approach for intervention in cancers and other proliferative diseases (Hojfeldt et al, Nat Rev Drug Discovery (2013), 12, 917-930; Pedersen and Helin, Trends in Cell Biology (2010), 20 (11), 662-671; McAllister et al, J. Med. Chem., (2016), 59 (4), 1308-1329; Thinnes et al, Biochimica et Biophysica Acta (2014), 1839(12), 1416-1432)

The KDM5/JARID1 family of histone demethylases are one of the sub-families of histone demethylases, and play a role in cancer and also contribute to the development of cancer resistance to chemotherapy (Rasmusson et al. Epigenomics (2014), 6(3), 277-286). The four KDM5 proteins (KDM5A/JARID1A, KDM5B/JARID1B, KDM5C/JARID1C and KDM5D/JARID1D) belong to the Jumonji domain histone demethylases and are responsible for the demethylation of trimethylated lysine 4 in histone H3 (H3K4me3), a mark for actively transcribed genes.

JARID1B is overexpressed in several cancers, including breast cancer (Yamamoto, Cancer Cell (2014), 25(6), 762-777; Yamane et al, Molecular Cell (2007), 25, 801-812), prostate cancer (Xiang et al, PNAS USA (2007), 104(49), 19226-19231), and lung cancer. In addition, JARID1B is required for mammary tumor formation in syngeneic or xenograft mouse models. Overexpression of JARID1B in hematopoietic stem cells in mice results in development of myeloid or B-lymphoid leukemia with complete penetrance (Ueda et al, Blood (2015), 125 (22), 3437-3446). A subset of melanoma cells are slow-cycling cells with doubling times of >4 weeks that depend on JARID1B (Roesch et, Cell (2010), 141(4), 583-594). Treatment of melanoma and other cancer cells with various drugs, including cisplatin and vemurafenib, uniformly leads to enrichment of slow-cycling, long-term tumor-maintaining melanoma cells expressing JARID1B, these are associated with increased self-renewal character (Roesch et al, Cancer Cell (2013), 23(6), 811-825). Furthermore, Jarid1a and Jarid1b contributes to retinoblastoma-mediated gene silencing during cellular senescence (Chicas et al PNAS USA. (2012), 109(23), 8971-8976)

Therefore, JARID1B represents an attractive target for cancer therapy.

Likewise KDM5A/JARID1A is overexpressed in a variety of human cancers, including lung cancer (Teng, Cancer Res (2013), 73(15), 4711-4721), and breast cancer (Cao et al, Cell Rep (2014), 6(5), 868-877). Cell culture and in vivo studies indicate that KDM5A and KDM5B contribute to cancer cell proliferation, drug resistance mechanisms, stem cell like properties and changes of cellular metabolism. There is also evidence that drug-tolerant cancer cells showed changes in “stemness” genes via epigenetic mechanisms involving molecules such as KDM5A (Sharma et al, Cell (2010), 141(1), 69-80; Yan et al, PloS One (2011), 6(9), e24397).

There is currently a need for compounds that inhibit of KDM5 demethylases for treating hyperproliferative diseases, preventing drug resistance, and/or for improving the efficacy of other cancer treatments (e.g. targeted therapies, chemotherapies, and radiotherapies.

Novel compounds and pharmaceutical compositions, certain of which have been found to inhibit KDM5 have been discovered, together with methods of synthesizing and using the compounds including methods for the treatment of KDM5-mediated diseases in a patient by administering the compounds.

In certain embodiments, compounds have structural Formula I:

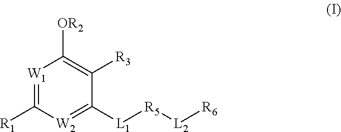

or a salt thereof, wherein:

W₁ and W₂ are independently chosen from N and CH;

R₁ is heteroaryl, which may be optionally substituted with one R₄ group;

R₂ is chosen from H and methyl;

R₃ is chosen from alkyl, cycloalkyl, haloalkyl and halocycloalkyl, any of which may be optionally substituted with one to three R^{x} groups;

R₄ is chosen from alkyl, amino, and heteroaryl, any of which may be optionally substituted with one to three R^{y} groups;

L₁ is chosen from a bond, O, alkyl, alkynyl, alkoxy, amino, alkylamino, amidoalkoxy, acyl, and acylalkoxy;

R₅ is null, halogen, or is chosen from alkyl, cycloalkyl, heterocycloalkyl, aryl, and heteroaryl, any of which may be optionally substituted with one to three R^{y} groups;

L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;

R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and

each R^{x}, R^{y}, and R^{z} is independently chosen from hydroxy, alkyl, alkoxy, halo, oxo, perhalomethyl, perhalomethoxy, cyano, and NH₂.

Certain compounds disclosed herein may possess useful KDM5 inhibiting activity, and may be used in the treatment or prophylaxis of a disease or condition in which KDM5 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting KDM5. Other embodiments provide methods for treating a KDM5-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of KDM5.

In certain embodiments, R₃ is C₁₋₄alkyl, C₃₋₄cycloalkyl and C₁₋₄haloalkyl.

In certain embodiments, R₃ is chosen from C₁₋₄alkyl and C₁₋₄haloalkyl.

In certain embodiments, R₃ is chosen from isopropyl, cyclopropyl, trifluoromethyl and ethyl.

In certain embodiments, R₃ isopropyl.

In certain embodiments, R₁ is heteroaryl optionally substituted with R₄.

In certain embodiments, R₁ is chosen from

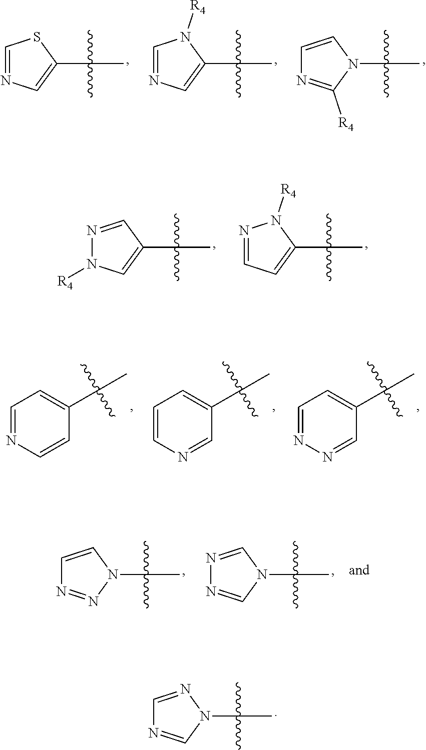

In certain embodiments, R₁ is chosen from

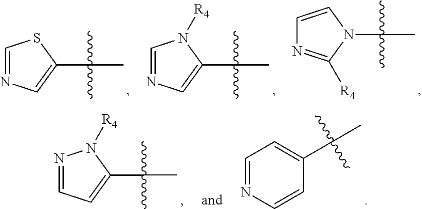

In certain embodiments, R₄ is chosen from alkyl and amino, any of which may be optionally substituted with one to three R_{z} groups.

In certain embodiments, R₄ is chosen from methyl, ethyl and amino, any of which may be optionally substituted with one to three R^{z} groups.

In certain embodiments, L₁ is chosen from a bond, O, C₁₋₄alkyl, C₁₋₄alkynyl, C₁₋₄alkoxy, amido, amidoC₁₋₄alkoxy, C₁₋₄alkylamidoC₁₋₄alkoxy, and acylC₁₋₄alkoxy.

In certain embodiments, L₂ is chosen from a bond, O, C₁₋₄alkyl, C₁₋₄alkoxy, C₁₋₄alkylamino, aminoC₁₋₄aklylamidoC₁₋₄alkyl, amido, C₁₋₄alkylamido, amidoC₁₋₄alkyl, C₁₋₄alkylamidoC₁₋₄alkyl, amidoC₁₋₄alkoxy, C₁₋₄alkylamidoC₁₋₄alkoxy, acyl, and acylC₁₋₄alkoxy.

In certain embodiments, compounds have structural Formula II:

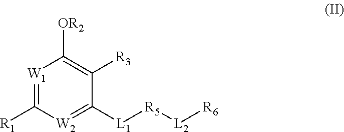

or a salt thereof, wherein:

W₁ and W₂ are independently chosen from N and CH;

R₁ is chosen from

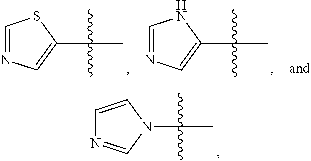

any of which may be optionally substituted with one R₄ group;

R₂ is chosen from H and methyl;

R₃ is chosen from isopropyl, cyclopropyl, trifluoromethyl and ethyl;

R₄ is chosen from alkyl, amino, and heteroaryl, any of which may be optionally substituted with one to three R^{x} groups;

L₁ is chosen from a bond, O, alkyl, alkynyl, alkoxy, amino, alkylamino, amidoalkoxy, acyl, and acylalkoxy;

R₅ is null, halogen, or is chosen from alkyl, cycloalkyl, heterocycloalkyl, aryl, and heteroaryl, any of which may be optionally substituted with one to three R^{y} groups;

L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;

R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and

each R^{x}, R^{y}, and R^{z} is independently chosen from hydroxy, alkyl, alkoxy, halo, oxo, perhalomethyl, perhalomethoxy, cyano, and NH₂.

In certain embodiments, compounds have structural Formula III:

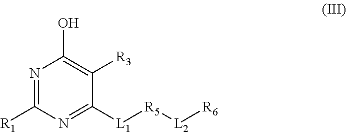

or a salt thereof, wherein:

R₁ is chosen from

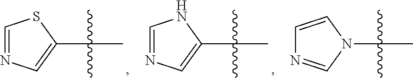 [SMILES: *c1cncs1 ; *c1cnc[nH]1 ; *n1ccnc1]

any of which may be optionally substituted with one R₄ group;

R₃ is chosen from isopropyl, cyclopropyl, trifluoromethyl and ethyl;

R₄ is chosen from alkyl, amino, and heteroaryl, any of which may be optionally substituted with one to three R^{x} groups;

L₁ is chosen from a bond, O, alkyl, alkynyl, alkoxy, amino, alkylamino, amidoalkoxy, acyl, and acylalkoxy;

R₅ is null, halogen, or is chosen from alkyl, cycloalkyl, heterocycloalkyl, aryl, and heteroaryl, any of which may be optionally substituted with one to three R^{y} groups;

L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;

R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{x} groups; and

each R^{x}, R^{y}, and R^{z} is independently chosen from hydroxy, alkyl, alkoxy, halo, oxo, perhalomethyl, perhalomethoxy, cyano, and NH₂.

In certain embodiments, compounds have structural Formula IV:

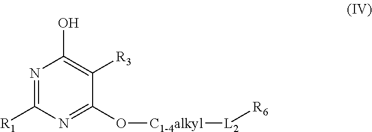

or a salt thereof, wherein:

R₁ is chosen from

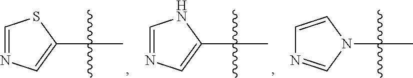 [SMILES: *c1cncs1 ; *c1cnc[nH]1 ; *n1ccnc1]

any of which may be optionally substituted with one R₄ group;

R₃ is chosen from isopropyl, cyclopropyl, trifluoromethyl and ethyl;

R₄ is chosen from alkyl, amino, and heteroaryl, any of which may be optionally substituted with one to three R^{x} groups;

L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;

R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and

each R^{x} and R^{z} is independently chosen from hydroxy, alkyl, alkoxy, halo, oxo, perhalomethyl, perhalomethoxy, cyano, and NH₂.

In certain embodiments, compounds have structural Formula V:

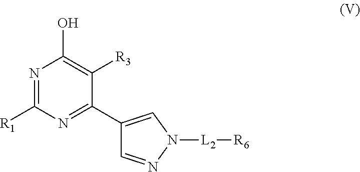

or a salt thereof, wherein:

R₁ is chosen from

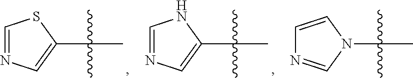 [SMILES: *c1cncs1 ; *c1cnc[nH]1 ; *n1ccnc1]

any of which may be optionally substituted with one R₄ group;

R₃ is chosen from isopropyl, cyclopropyl, trifluoromethyl and ethyl;

R₄ is chosen from alkyl, amino, and heteroaryl, any of which may be optionally substituted with one to three R^{x} groups;

L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;

R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and

each R^{x} and R^{z} is independently chosen from hydroxy, alkyl, alkoxy, halo, oxo, perhalomethyl, perhalomethoxy, cyano, and NH₂.

In certain embodiments, compounds have structural Formula VI:

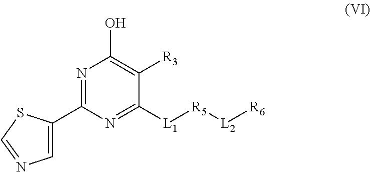

or a salt thereof; wherein:

R₃ is chosen from isopropyl, cyclopropyl, trifluoromethyl and ethyl;

L₁ is chosen from a bond, O, alkyl, alkynyl, alkoxy, amino, alkylamino, amidoalkoxy, acyl, and acylalkoxy;

R₅ is null, halogen, or is chosen from alkyl, cycloalkyl, heterocycloalkyl, aryl, and heteroaryl, any of which may be optionally substituted with one to three R^{y} groups;

L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;

R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and

each R^{y} and R^{z} is independently chosen from hydroxy, alkyl, alkoxy, halo, oxo, perhalomethyl, perhalomethoxy, cyano, and NH₂.

In certain embodiments, compounds have structural Formula VII:

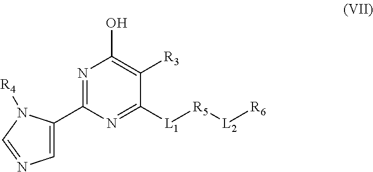

or a salt thereof; wherein

R₃ is chosen from isopropyl, cyclopropyl, trifluoromethyl and ethyl;

R₄ is chosen from alkyl, amino, and heteroaryl, any of which may be optionally substituted with one to three R^{x} groups;

L₁ is chosen from a bond, O, alkyl, alkynyl, alkoxy, amino, alkylamino, amidoalkoxy, acyl, and acylalkoxy;

R₅ is null, halogen, or is chosen from alkyl, cycloalkyl, heterocycloalkyl, aryl, and heteroaryl, any of which may be optionally substituted with one to three R^{y} groups;

L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;

R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and

each R^{x}, R^{y}, and R^{z} is independently chosen from hydroxy, alkyl, alkoxy, halo, oxo, perhalomethyl, perhalomethoxy, cyano, and NH₂.

In certain embodiments, compounds have structural Formula VIII:

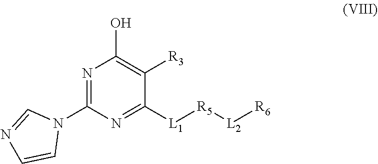

or a salt thereof; wherein:

R₃ is chosen from isopropyl, cyclopropyl, trifluoromethyl and ethyl;

L₁ is chosen from a bond, O, alkyl, alkynyl, alkoxy, amino, alkylamino, amidoalkoxy, acyl, and acylalkoxy;

R₅ is null, halogen, or is chosen from alkyl, cycloalkyl, heterocycloalkyl, aryl, and heteroaryl, any of which may be optionally substituted with one to three R^{y} groups;

L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;

R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and

each R^{y} and R^{z} is independently chosen from hydroxy, alkyl, alkoxy, halo, oxo, perhalomethyl, perhalomethoxy, cyano, and NH₂.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

Also provided is a compound chosen from the Examples disclosed herein.

The present invention also relates to a method of inhibiting at least one KDM5 function comprising the step of contacting KDM5 with a compound as described herein. The cell phenotype, cell proliferation, activity of KDM5, change in biochemical output produced by active KDM5, expression of KDM5, or binding of KDM5 with a natural binding partner may be monitored. Such methods may be modes of treatment of disease, biological assays, cellular assays, biochemical assays, or the like.

Also provided herein is a method of treatment of a KDM5-mediated disease comprising the administration of a therapeutically effective amount of a compound as disclosed herein, or a salt thereof, to a patient in need thereof.

In certain embodiments, the disease is cancer.

In some embodiments, the compounds of the present disclosure may be used to prevent or treat cancer, wherein the cancer is one or a variant of Acute Lymphoblastic Leukemia (ALL), Acute Myeloid Leukemia (AML), Adrenocortical Carcinoma, AIDS-Related Cancers (Kaposi Sarcoma and Lymphoma), Anal Cancer, Appendix Cancer, Atypical Teratoid/Rhabdoid Tumor, Basal Cell Carcinoma, Bile Duct Cancer (including Extrahepatic), Bladder Cancer, Bone Cancer (including Osteosarcoma and Malignant Fibrous Histiocytoma), Brain Tumor (such as Astrocytomas, Brain and Spinal Cord Tumors, Brain Stem Glioma, Central Nervous System Atypical Teratoid/Rhabdoid Tumor, Central Nervous System Embryonal Tumors, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma), Breast Cancer, Bronchial Tumors, Burkitt Lymphoma, Basal Cell Carcinoma, Bile Duct Cancer (including Extrahepatic), Bladder Cancer, Bone Cancer (including Osteosarcoma and Malignant Fibrous Histiocytoma), Carcinoid Tumor, Carcinoma of Unknown Primary, Central Nervous System (such as Atypical Teratoid/Rhabdoid Tumor, Embryonal Tumors and Lymphoma), Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma (Mycosis Fungoides and Sézary Syndrome), Duct, Bile (Extrahepatic), Ductal Carcinoma In Situ (DCIS), Embryonal Tumors (Central Nervous System), Endometrial Cancer, Ependymoblastoma, Ependymoma, Esophageal Cancer, Esthesioneuroblastoma, Ewing Sarcoma Family of Tumors, Extracranial Germ Cell Tumor, Extragonadal Germ Cell Tumor, Extrahepatic Bile Duct Cancer, Eye Cancer (like Intraocular Melanoma, Retinoblastoma), Fibrous Histiocytoma of Bone (including Malignant and Osteosarcoma) Gallbladder Cancer, Gastric (Stomach) Cancer, Gastrointestinal Carcinoid Tumor, Gastrointestinal Stromal Tumors (GIST), Germ Cell Tumor (Extracranial, Extragonadal, Ovarian), Gestational Trophoblastic Tumor, Glioma, Hairy Cell Leukemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors (Endocrine, Pancreas), Kaposi Sarcoma, Kidney (including Renal Cell), Langerhans Cell Histiocytosis, Laryngeal Cancer, Leukemia (including Acute Lymphoblastic (ALL), Acute Myeloid (AML), Chronic Lymphocytic (CLL), Chronic Myelogenous (CML), Hairy Cell), Lip and Oral Cavity Cancer, Liver Cancer (Primary), Lobular Carcinoma In Situ (LCIS), Lung Cancer (Non-Small Cell and Small Cell), Lymphoma (AIDS-Related, Burkitt, Cutaneous T-Cell (Mycosis Fungoides and Sézary Syndrome), Hodgkin, Non-Hodgkin, Primary Central Nervous System (CNS), Macroglobulinemia, Waldenström, Male Breast Cancer, Malignant Fibrous Histiocytoma of Bone and Osteosarcoma, Medulloblastoma, Medulloepithelioma, Melanoma (including Intraocular (Eye)), Merkel Cell Carcinoma, Mesothelioma (Malignant), Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma and Multiple Myeloma, Myeloproliferative Disorders (Chronic), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer (such as Epithelial, Germ Cell Tumor, and Low Malignant Potential Tumor), Pancreatic Cancer (including Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (like Ewing Sarcoma Family of Tumors, Kaposi, Soft Tissue, Uterine), Sézary Syndrome, Skin Cancer (such as Melanoma, Merkel Cell Carcinoma, Nonmelanoma), Small Cell Lung Cancer, Small Intestine Cancer, Soft Tissue Sarcoma, Squamous Cell Carcinoma, Squamous Neck Cancer with Occult Primary, Metastatic, Stomach (Gastric) Cancer, Supratentorial Primitive Neuroectodermal Tumors, T-Cell Lymphoma (Cutaneous, Mycosis Fungoides and Sézary Syndrome), Testicular Cancer, Throat Cancer, Thymoma and Thymic Carcinoma, Thyroid Cancer, Transitional Cell Cancer of the Renal Pelvis and Ureter, Trophoblastic Tumor (Gestational), Unknown Primary, Unusual Cancers of Childhood, Ureter and Renal Pelvis, Transitional Cell Cancer, Urethral Cancer, Uterine Cancer, Endometrial, Uterine Sarcoma, Waldenström Macroglobulinemia or Wilms Tumor.

Also provided herein is a compound as disclosed herein for use as a medicament.

Also provided herein is a compound as disclosed herein for use as a medicament for the treatment of a KDM5-mediated disease.

Also provided is the use of a compound as disclosed herein as a medicament.

Also provided is the use of a compound as disclosed herein as a medicament for the treatment of a KDM5-mediated disease.

Also provided is a compound as disclosed herein for use in the manufacture of a medicament for the treatment of a KDM5-mediated disease.

Also provided is the use of a compound as disclosed herein for the treatment of a KDM5-mediated disease.

Also provided herein is a method of inhibition of KDM5 comprising contacting KDM5 with a compound as disclosed herein, or a salt thereof.

Also provided herein is a method for achieving an effect in a patient comprising the administration of a therapeutically effective amount of a compound as disclosed herein, or a salt thereof, to a patient, wherein the effect is chosen from cognition enhancement.

In certain embodiments, compounds disclosed herein are selective for KDM5 isoforms over other KDM over other classes of KDM, such as KDM1, KDM2, KDM3, KDM4, KDM6, and KDM7. Additionally, compounds disclosed herein may be selective amongst the KDM5 isoforms KDM5A, KDM5B, KDM5C and KDM5D in various ways. For example, compounds described herein may be pan-inhibitors of all the isoforms, or be selective for only one isoform, for example KDM5B.

In certain embodiments, the KDM5-mediated disease is cancer.

In certain embodiments, said cancer is squamous cell carcinoma.

In certain embodiments, the squamous cell carcinoma is located in the skin, lips, mouth, esophagus, urinary bladder, prostate, lungs, vagina, or cervix.

In certain embodiments, said cancer is chosen from leukemia, lymphoma, oral cancer, laryngeal cancer, esophageal cancer, prostate cancer, bladder cancer, renal cancer, uterine cancer, ovarian cancer, testicular cancer, rectal cancer, colon cancer, lung cancer, brain cancer, breast cancer, pancreatic cancer, stomach cancer, liver cancer, thyroid cancer, melanoma, and multiple myeloma.

Also provided is a method of modulation of a KDM5-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral

In certain embodiments, the pharmaceutical composition is formulated for parenteral administration.

In certain embodiments, the oral pharmaceutical composition is chosen from a tablet and a capsule.

### DETAILED DESCRIPTION

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)-group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group—with R and R′ as defined herein.

The term “N-carbamyl” as used herein, alone or in combination, refers to a ROC(O)NR′-group, with R and R′ as defined herein.

The term “carbonyl,” as used herein, when alone includes formyl [—C(O)H] and in combination is a —C(O)-group.

The term “carboxyl” or “carboxy,” as used herein, refers to —C(O)OH or the corresponding “carboxylate” anion, such as is in a carboxylic acid salt. An “O-carboxy” group refers to a RC(O)O-group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, pyrrolidinonyl, tetrahydropyridinyl, piperidinyl, piperidinonyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen and lower alkyl, either of which may be optionally substituted.

The term “mercaptyl” as used herein, alone or in combination, refers to an RS-group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to a —S(O)₂—, —S(O)₂R, or —S(O)₂R— group, with R as defined herein.

The term “sulfonamido,” as used herein, alone or in combination, includes both N-sulfonamido and S-sulfonamido. The term “N-sulfonamido” refers to either a RS(O)₂NR′— or —S(O)₂NR′— group with R and R′ as defined herein. The term “S-sulfonamido” refers to a —S(O)₂NRR′ or —S(O)₂NR— group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. For example, keto-enol tautomers are provided; where one form is drawn or named, the other is provided as well. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“KDM5 inhibitor” is used herein to refer to a compound that exhibits an IC₅₀ with respect to KDM5 activity of no more than about 100 μM and more typically not more than about 50 μM, as measured in the KDM5 assay described generally herein. “IC₅₀” is that concentration of inhibitor which reduces the activity of an enzyme (e.g., KDM5) to half-maximal level. Certain compounds disclosed herein have been discovered to exhibit inhibition against KDM5. In certain embodiments, compounds will exhibit an IC₅₀ with respect to KDM5 of no more than about 10 μM; in yet further embodiments, compounds will exhibit an IC₅₀ with respect to KDM5 of not more than about 1 μM; in yet further embodiments, compounds will exhibit an IC₅₀ with respect to KDM5 of not more than about 100 nM, as measured in the KDM5 assay described herein; in yet further embodiments, compounds will exhibit an IC₅₀ with respect to KDM5 of not more than about 50 nM, as measured in the KDM5 assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

### Compounds

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

### Pharmaceutical Compositions

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

### Oral Administration

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

### Parenteral Administration

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

### Rectal, Buccal, and Sublingual Administration

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

### Topical Administration

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

### Administration by Inhalation

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with anti-cancer (chemotherapeutic) drugs. Classes of anti-cancer drugs include, but are not limited to: alkylating agents, anti-metabolites, antimitotics, checkpoint inhibitors, plant alkaloids and terpenoids, topoisomerase inhibitors, cytotoxic antibiotics, aromatase inhibitors, angiogenesis inhibitors, anti-steroids and anti-androgens, mTOR inhibitors, tyrosine kinase inhibitors, and others.

For use in cancer and neoplastic diseases a KDM5/JARID1 inhibitor may be optimally used together with one or more of the following non-limiting examples of anti-cancer agents:
(1) alkylating agents, including but not limited to carmustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, and cyclophosphamide (ENDOXAN);
(2) anti-metabolites, including but not limited to cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX), raltitrexed;
(3) antimitotics, which are often plant alkaloids and terpenoids, or derivateves thereof, including but not limited to taxanes such as docetaxel (TAXITERE) and paclitaxel (ABRAXANE, TAXOL); vinca alkaloids such as vincristine (ONCOVIN), vinblastine, vindesine, and vinorelbine (NAVELBINE);
(4) topoisomerase inhibitors, including but not limited to camptothecin (CTP), irinotecan (CAMPTOSAR), topotecan (HYCAMTIN), teniposide (VUMON), and etoposide (EPOSIN);
(5) cytotoxic antibiotics, including but not limited to actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOVANTRONE), plicamycin;
(6) aromatase inhibitors, including but not limited to aminoglutethimide, anastrozole (ARIMIDEX), letrozole (FEMARA), vorozole (RIVIZOR), exemestane (AROMASIN);
(7) angiogenesis inhibitors, including but not limited to genistein, sunitinib (SUTENT) and bevacizumab (AVASTIN);
(8) anti-steroids and anti-androgens such as aminoglutethimide (CYTADREN), bicalutamide (CASODEX), cyproterone, flutamide (EULEXIN), nilutamide (NILANDRON);
(9) tyrosine kinase inhibitors, including but not limited to imatinib (GLEEVEC), erlotinib (TARCEVA), lapatininb (TYKERB), sorafenib (NEXAVAR), and axitinib (INLYTA);
(10) mTOR inhibitors such as everolimus, temsirolimus (TORISEL), and sirolimus;
(11) monoclonal antibodies such as trastuzumab (HERCEPTIN) and rituximab (RITUXAN);
(12) other agents, such as amsacrine; Bacillus Calmette-Guérin (B—C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating KDM5-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of KDM5-mediated disorders.

Specific diseases to be treated by the compounds, compositions, and methods disclosed herein include cancer.

Specific cancers to be treated by the compounds, compositions, and methods disclosed herein include squamous cell carcinoma.

Specific cancers to be treated by the compounds, compositions, and methods disclosed herein also include leukemia, lymphoma, oral cancer, laryngeal cancer, esophageal cancer, prostate cancer, bladder cancer, renal cancer, uterine cancer, ovarian cancer, testicular cancer, rectal cancer, colon cancer, lung cancer, brain cancer, breast cancer, pancreatic cancer, stomach cancer, liver cancer, thyroid cancer, melanoma, and multiple myeloma.

Cancers to be treated by the methods disclosed herein include colon cancer, breast cancer, ovarian cancer, lung cancer and prostrate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic. Additional types of cancers which may be treated using the compounds and methods of the invention include, but are not limited to, adrenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### List of Abbreviations

Ac₂O=acetic anhydride; AcCl=acetyl chloride; AcOH=acetic acid; AIBN=azobisisobutyronitrile; aq.=aqueous; BAST=bis(2-methoxyethyl)aminosulfur trifluoride; Bu=butyl; Bu₃SnH=tributyltin hydride; CD₃OD=deuterated methanol; CDCl₃=deuterated chloroform; CDI=1,1′-carbonyldiimidazole; DAST=(diethylamino)sulfur trifluoride; dba=dibenzylideneacetone DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethyl-formamide; DMSO-d₆=deuterated dimethyl sulfoxide; DMSO=dimethyl sulfoxide; DPPA=diphenylphosphoryl azide; dppf=1,1′-bis(diphenylphosphino)ferrocene; EDC.HCl=EDCI.HCl=1-ethyl-3-(3-dimethylaminopropyl)carbodiimide hydrochloride; Et=ethyl; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyl-disilazane; HOBT=1-hydroxybenzotriazole; i-Pr=isopropyl=2-propyl; i-PrOH=isopropanol; LAH=lithium aluminiumhydride; LDA=lithium diisopropyl amide; LiHMDS=Lithium bis(trimethylsilyl)amide; MeCN=acetonitrile; MeI=methyl iodide; MeOH=methanol; MP-carbonate resin=macroporous triethylammonium methylpolystyrene carbonate resin; MsCl=mesyl chloride; MTBE=methyl tertiary butyl ether; n-BuLi=n-butyllithium; NaHMDS=Sodium bis(trimethylsilyl)amide; NaOEt=sodium ethoxide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(0); Pd₂(dba)₃=tris(dibenzylideneacetone)dipalladium(0); PdCl₂(PPh₃)₂=bis(triphenylphosphine)palladium(II) dichloride; PG=protecting group; Ph=phenyl; prep-HPLC=preparative high-performance liquid chromatography; PMBCl=para-methoxybenzyl; PMBCl=para-methoxybenzyl chloride; PMBOH=para-methoxybenzyl alcohol; PyBop=(benzotriazol-1-yloxy)tripyrrolidino-phosphonium hexafluorophosphate; Pyr=pyridine; RT=room temperature; RuPhos=2-dicyclohexylphosphino-2′,6′-diisopropoxybiphenyl; sat.=saturated; ss=saturated solution; tBu=t-Bu=tert-butyl=1,1-dimethylethyl; TBDPS=t-butyldiphenylsilyl; t-BuOH=tert-butanol; T3P=Propylphosphonic Anhydride; TEA=Et₃N=triethylamine; TFA=trifluoroacetic acid; TFAA=trifluoroacetic anhydride; THF=tetrahydrofuran; TIPS=triisopropylsilyl; Tol=toluene; TsCl=tosyl chloride; Trt=trityl=(triphenyl)methyl; Xantphos=4,5-Bis(diphenylphosphino)-9,9-dimethylxanthene; XPhos=2-dicyclohexylphosphino-2′,4′,6′-triisopropylbiphenyl.

### General Synthetic Schemes for Examples

In Scheme 1 and elsewhere, it is understood that hydroxy pyrimidines may exist in keto/enol tautomeric equilibrium. For brevity, only one of the possible structures is shown for several of these hydroxy pyrimidine species.

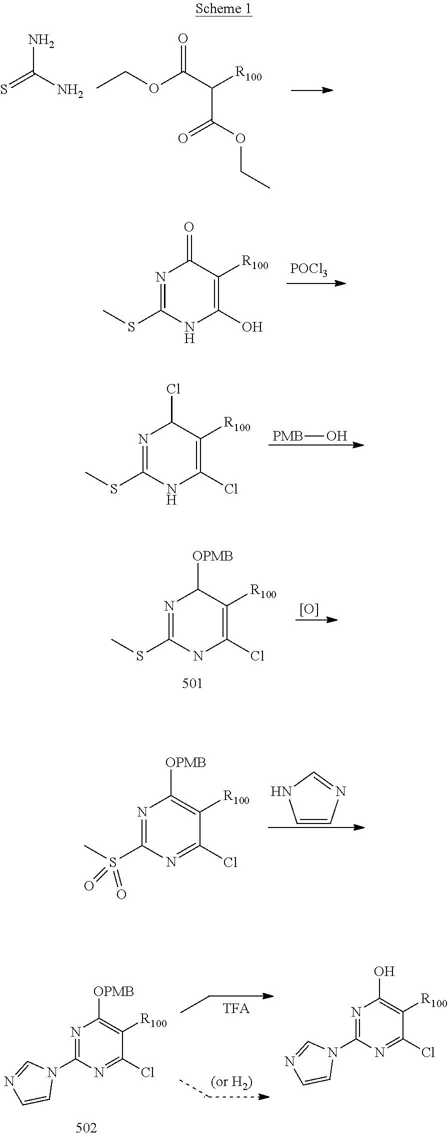

Example 1, and similar compounds, can be synthesized using the general synthetic procedure set forth in Scheme 1. Substituted malonic ester and thiourea are combined to give the pyrimidine core. Conversion to the dichloro compound is accomplished with phosphorus oxychloride. The PMB ether 501 is formed by displacement, under basic conditions, with p-methoxybenzyl alcohol (“PMBOH”). The thioether functionality is oxidized to sulfone, and displacement with nucleophilic imidazole gives the mono-chloro product 502. Finally, the PMB ether is cleaved with TFA. An alternative method for cleavage of the PMB ether, here and in the remaining schemes, is with catalytic hydrogenolysis.

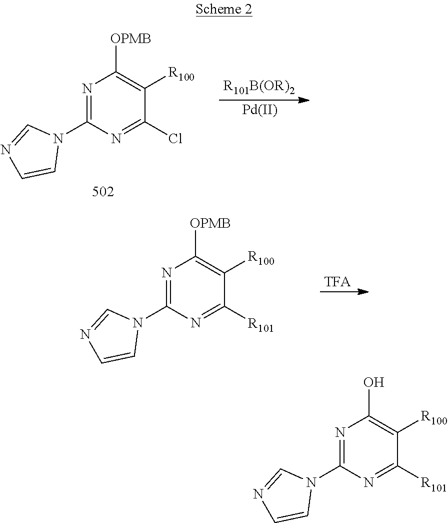

Example 2 and Example 3, and similar compounds, can be synthesized using the general synthetic procedure set forth in Scheme 2. Mono-chloro compound 502, from Scheme 1, is coupled under Suzuki conditions with a suitable boronic acid or boronic ester. Next, the PMB group is cleaved with TFA.

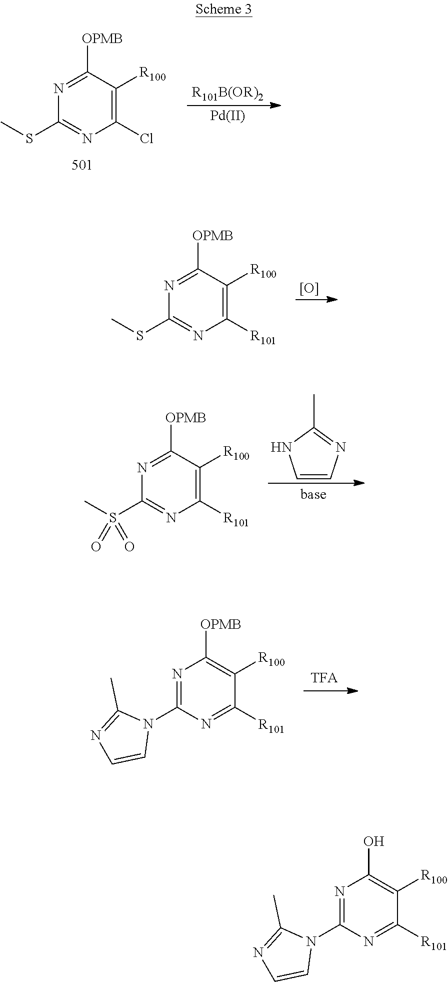

Example 4, and similar compounds, can be synthesized using an alternate synthetic procedure set forth in Scheme 3. The starting material 501, obtained from Scheme 1, is coupled with an appropriate boronic ester under Suzuki conditions. The sulfide is oxidized to a sulfone, and the sulfone is then displaced with nucleophilic imidazole. The PMB ether group is cleaved with acid.

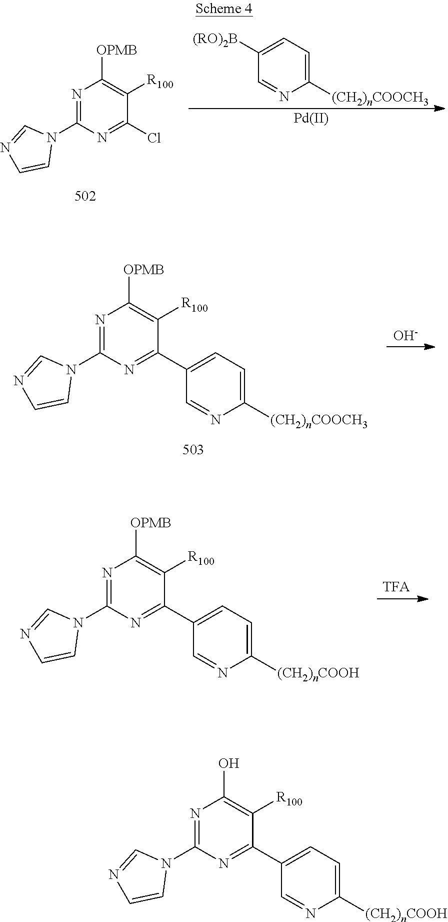

Example 5, and similar compounds, can be synthesized using the procedure shown in Scheme 4. The starting material 502, obtained from Scheme 1, is coupled under Suzuki conditions with the carboxylic ester shown, affording coupled product 503. The ester moiety of the product is cleaved with hydroxide, and the PMB ether is then cleaved with TFA.

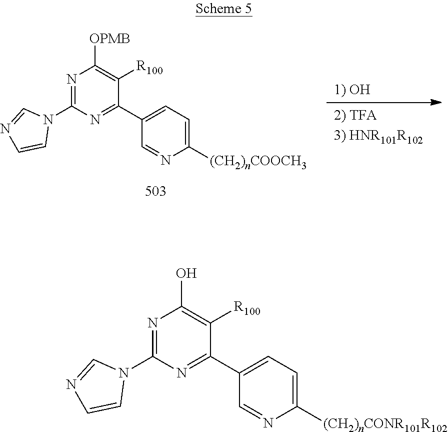

Example 6 and Example 7 and Example 8 and Example 9, and similar compounds, can be synthesized using the procedure shown in Scheme 5. Intermediate 503, obtained from Scheme 3, is treated with hydroxide to afford the corresponding carboxylic acid. The compound is then treated with TFA, to cleave the PMB ether. Finally, the carboxylic acid functionality is coupled with an amine under conventional conditions to yield the amide product.

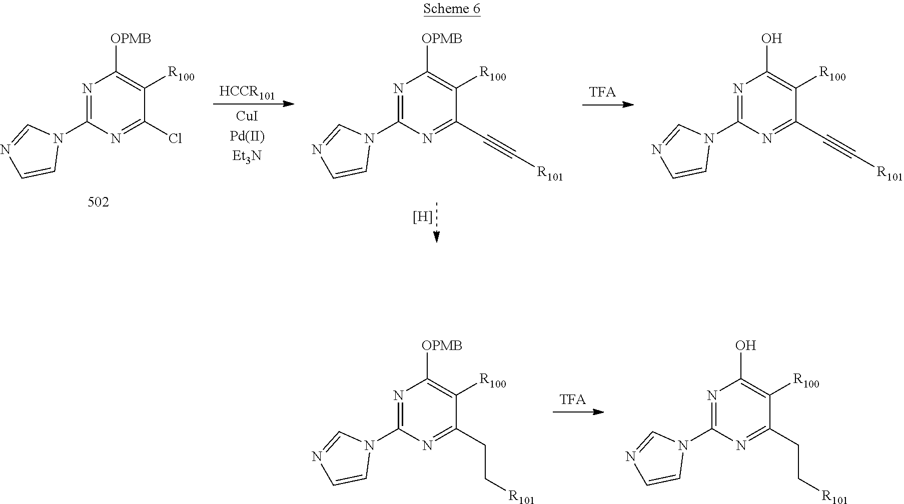

Example 10a/b, and similar compounds, can be synthesized using the procedure shown in Scheme 6. The starting material 502, obtained from Scheme 1, is subjected to Sonogashira coupling conditions with a terminal alkyne. The alkyne can be optionally hydrogenated at this step. The PMB ether is then cleaved with TFA.

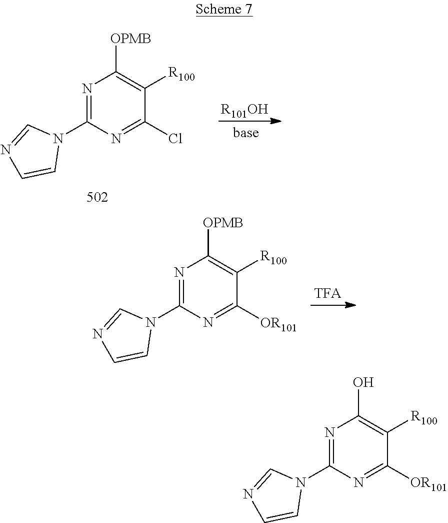

Example 11 and Example 12, and similar compounds, can be synthesized using the procedure shown in Scheme 7. The starting material 502, obtained from Scheme 1, is reacted with the alcohol shown, under basic conditions. The PMB ether is then cleaved with TFA.

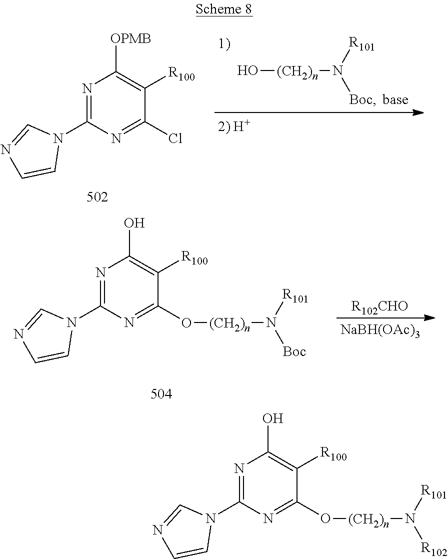

Example 13, and similar compounds, can be synthesized using the procedure shown in Scheme 8. The starting material 502, obtained from Scheme 1, is reacted under basic conditions with a Boc-protected amino alcohol, represented here as HO—(CH₂)ₙ—N(Boc)(R₁₀₁), to give the ether product. Treatment with acid removes both the Boc group and the PMB group to give 504. Reductive amination conditions are then used to couple this amine moiety with an aldehyde, giving the indicated product.

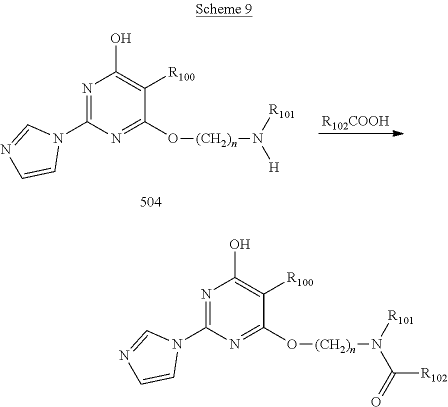

Example 14, and similar compounds, can be synthesized using the procedure shown in Scheme 9. The starting material, 504, obtained from Scheme 8, is subjected to standard reagents to couple the amine moiety with a carboxylic acid, giving the indicated product.

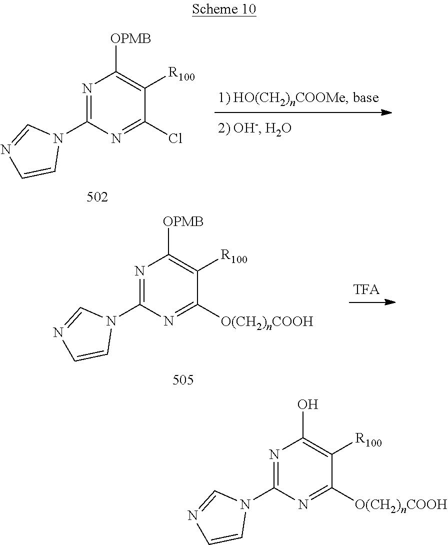

Example 15, and similar compounds, can be synthesized using the procedure shown in Scheme 10. The starting material 502, obtained from Scheme 1, is reacted under basic conditions with a hydroxy ester, represented here as HO(CH₂)ₙCOOMe. The ester is cleaved with hydroxide, giving carboxylic acid 505. The PMB ether is then cleaved with TFA.

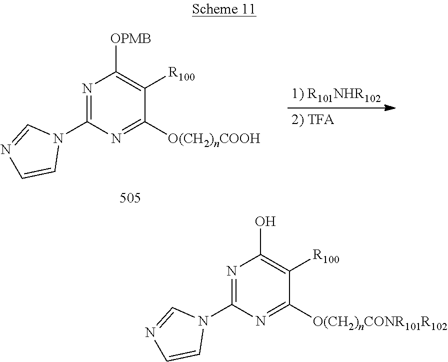

Example 16 and Example 17, and similar compounds, can be synthesized using the procedure shown in Scheme 11. Carboxylic acid 505 is coupled with an amine to form an amide using conventional techniques. The PMB ether is then cleaved with TFA.

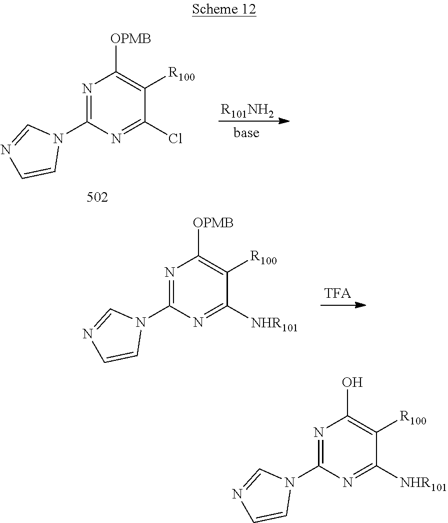

Example 18, and similar compounds, can be synthesized using the procedure shown in Scheme 12. The starting material 502, obtained from Scheme 1, is reacted under basic conditions with an amine to form the aminopyrimidine shown. The PMB ether is then cleaved with TFA.

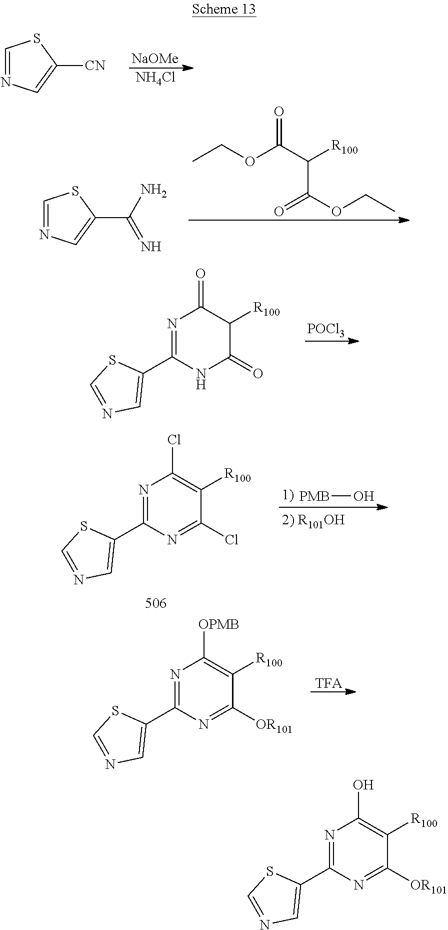

Example 19, and similar compounds, can be synthesized using an alternate synthetic procedure set forth in Scheme 13. The pyrimidine core is formed by condensation of a substituted carboximidamide with a substituted malonic ester. Conversion to the dichloro compound 506 is accomplished with phosphorus oxychloride. Reaction with a single equivalent of p-methoxybenzyl alcohol gives the PMB ether. Reaction with a second alcohol gives a differentially substituted compound. The PMB ether is then cleaved with TFA.

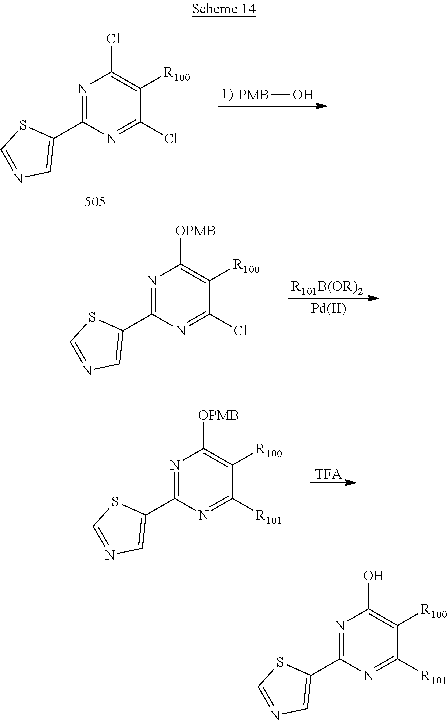

Example 20, and similar compounds, can be synthesized using an alternate synthetic procedure set forth in Scheme 14. Intermediate 506, from Scheme 13 is reacted under Suzuki conditions with a suitable boronic ester to give the coupled product. The PMB ether is then cleaved with TFA.

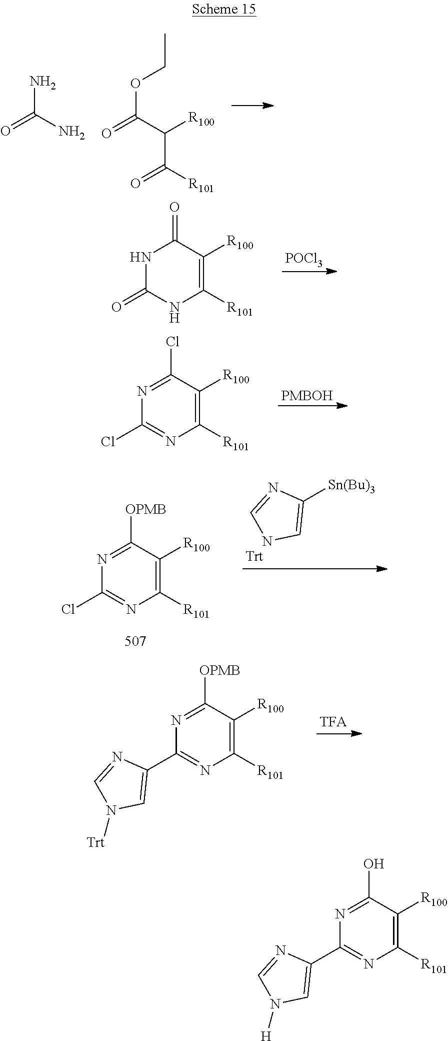

Example 21, and similar compounds, can be synthesized using the procedure shown in Scheme 15. Substituted acetoacetic ester and urea are combined to give the pyrimidinedione core. Conversion to the dichloro compound is accomplished with phosphorus oxychloride. The mono-ether is formed by displacement, under basic conditions, with PMBOH. The resulting monochloro compound 507 is coupled under with an organostannane. The PMB ether is then cleaved with TFA.

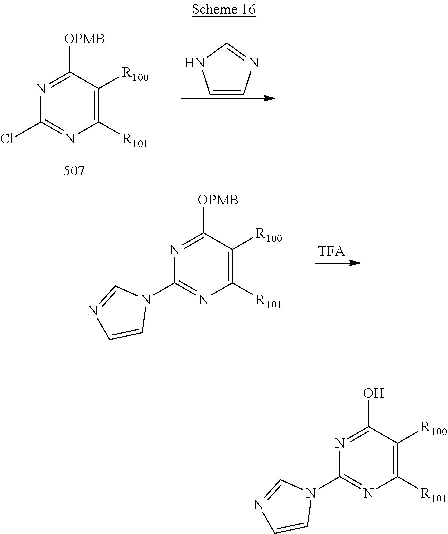

Examples 22, 23, 24a/b, and 25, and similar compounds, can be synthesized using the procedure shown in Scheme 16. Intermediate 507, obtained from Scheme 15, is reacted with imidazole to form the coupled product shown. This coupling reaction is optionally mediated by base or transition metal catalyst. The PMB ether is then cleaved with TFA.

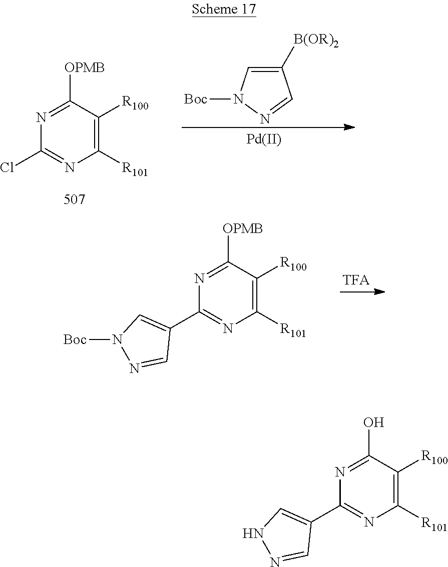

Example 26, and similar compounds, can be synthesized using an alternate synthetic procedure set forth in Scheme 17. Intermediate 507, obtained from Scheme 15, is coupled under Suzuki conditions with an arylboronic ester. Treatment of the product with acid serves to cleave both the PMB ether and the Boc carbamate protecting group.

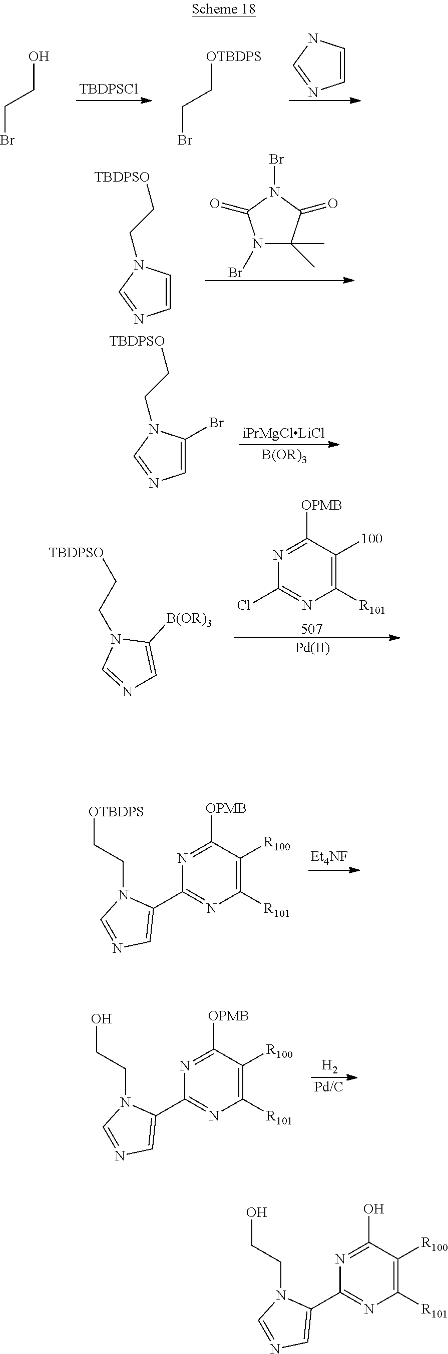

Example 27, and similar compounds, can be synthesized using an alternate synthetic procedure set forth in Scheme 18. Bromoethanol is protected as a silyl ether, then coupled with imidazole, which is then oxidatively brominated. The bromo functionality is then converted to a boronic ester, which is coupled under Suzuki conditions with intermediate 507, obtained from Scheme 15. Finally, deprotection is carried out in two steps: reaction with fluoride anion cleaves the silyl ether, then hydrogenolysis cleaves the PMB ether.

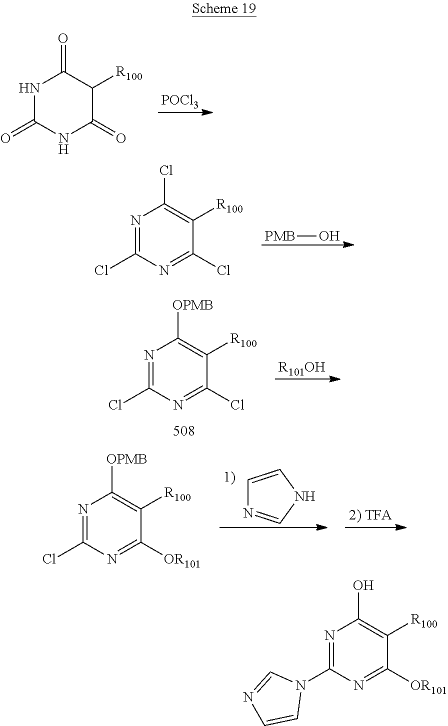

Example 28 and Example 29, and similar compounds, can be synthesized using an alternate synthetic procedure set forth in Scheme 19. Substituted barbituric acid is converted to the trichloro compound with phosphorus oxychloride. Reaction with a single equivalent of p-methoxybenzyl alcohol gives the PMB ether 508. Reaction with a second alcohol gives a differentially substituted compound. The third chlorine is then displaced by reaction with imidazole. The PMB ether is then cleaved with TFA.

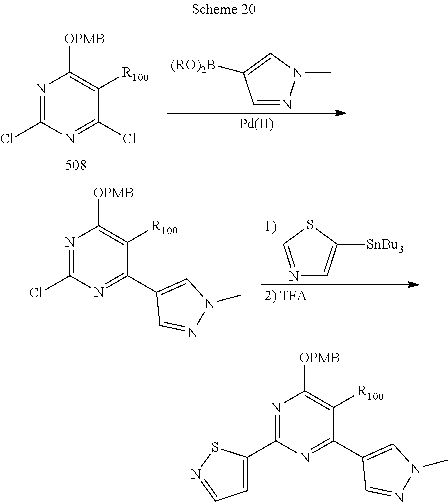

Example 30, and similar compounds, can be synthesized using an alternate synthetic procedure set forth in Scheme 20. Intermediate 508, from Scheme 19, is reacted under Suzuki conditions with a suitable boronic ester reagent to provide the substituted product. A second organometallic coupling reaction, here shown as a Stille coupling, is used to substitute the remaining chloride. The PMB ether is then cleaved with TFA.

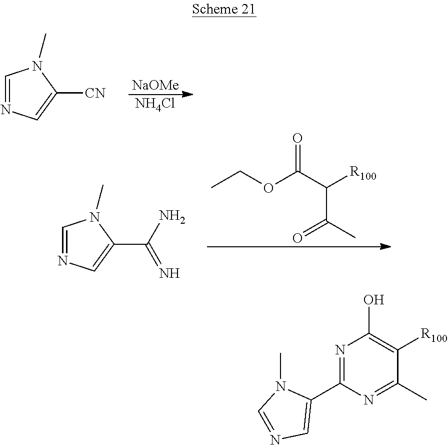

Example 31, and similar compounds, can be synthesized using an alternate synthetic procedure set forth in Scheme 21. The nitrile starting material is reacted with ammonia under strongly basic conditions to form the carboximidamide shown. The pyrimidine core is then formed directly through condensation with a suitable acetoacetic ester.

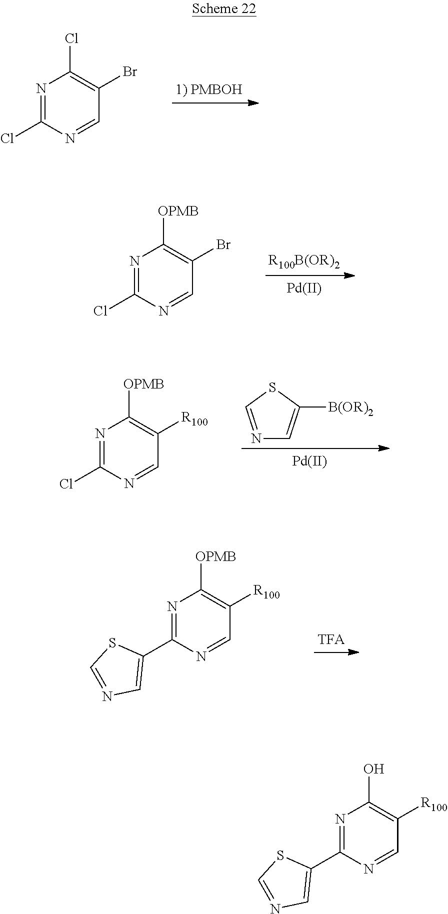

Example 32, and similar compounds, can be synthesized using an alternate synthetic procedure set forth in Scheme 22. Reaction with a single equivalent of p-methoxybenzyl alcohol with the commercially available triply halogenated pyrimidine gives the PMB ether. Regioselective coupling with a suitable boronic ester is achieved under Suzuki conditions. Displacement of the remaining halogen can be accomplished with a second Suzuki coupling. The PMB ether is then cleaved with TFA.

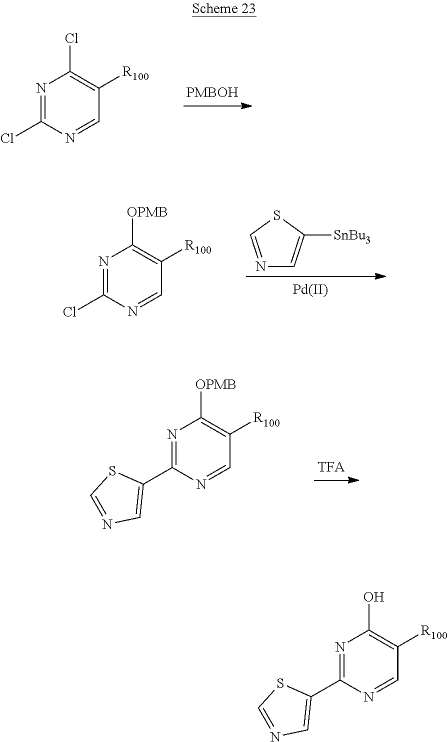

Example 33, and similar compounds, can be synthesized using an alternate synthetic procedure set forth in Scheme 23. Reaction with a single equivalent of PMBOH with the doubly halogenated pyrimidine shown gives the PMB ether. Displacement of the remaining halogen can be accomplished by Pd(II) mediated coupling with an organostannane. The PMB ether is then cleaved with TFA.

The invention is further illustrated by the following examples.

### Example 1

### 2-(1H-imidazol-1-yl)-5-isopropyl-6-(1-isopropyl-1H-pyrazol-4-yl)pyrimidin-4-ol

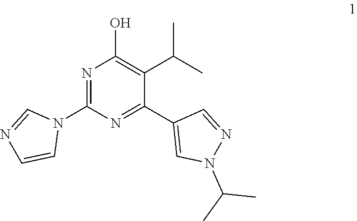 [SMILES: CC(C)c1c(O)nc(-n2ccnc2)nc1-c1cnn(C(C)C)c1]

### Step 1: 6-hydroxy-5-isopropyl-2-(methylthio)pyrimidin-4(1H)-one

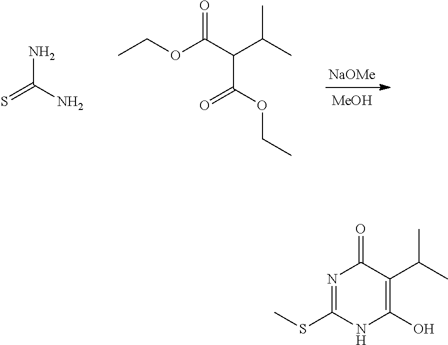

To a solution of thiourea (1.71 g, 22.5 mmol) in MeOH (15 mL) was added freshly prepared NaOMe (NaH in 60% in mineral oil (0.99 g, 24.8 mmol) and MeOH (5 mL) were stirred at RT for 15 min). To this mixture diethyl 2-isopropylmalonate (4.56 g, 22.5 mmol) was added in MeOH (15 mL). The resulting mixture was heated at reflux overnight. The reaction was allowed to cool to RT, additional NaOMe (NaH (60% in mineral oil; 0.99 g, 24.8 mmol) and MeOH (5 mL)) was added and the reaction was heated at reflux for 1.5 h. The reaction was allowed to cool to RT, MeI (1.55 mL, 24.8 mmol) was added and the reaction was heated at reflux overnight. The reaction was concentrated, ice-water was added and then acidified with cone HCl. The resulting mixture was filtered, washed with water, diethyl ether, and hexanes to give the product (3.78 g, 18.9 mmol, 84% yield) as a white solid.

MS (ES⁺) C₈H₁₂N₂O₂S requires: 200, found: 201 [M+H]⁺.

### Step 2: 4,6-dichloro-5-isopropyl-2-(methylthio)pyrimidine

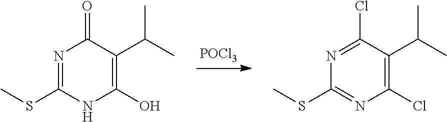

To a flask containing the material from the previous step (3.78 g, 18.9 mmol) was added POCl₃ (12 mL, 129 mmol) and the resulting mixture was stirred at 105° C. for 14 h. The black reaction was cooled to RT, concentrated, dissolved in DCM (75 mL) and poured into a mixture of ice and saturated NaHCO₃ (150 mL). The mixture was stirred until all of the ice melted and evolution of gas stopped and the layers were separated. To the aqueous phase was added saturated NaCl and then extracted with DCM (3×75 mL). The organic layers were combined, washed with brine, dried over MgSO₄, filtered, and concentrated to afford a black oil. The crude product was purified by chromatography (40 g Redisep, 0 to 20% EtOAc/hexanes) to give the product (3.8 g, 16.0 mmol, 85% yield) as an off-white solid/wax.

MS (ES⁺) C₈H₁₀C₁₂N₂S requires: 235, found: 236 [M+H]⁺.

### Step 3: 4-chloro-5-isopropyl-6-((4-methoxybenzyl)oxy)-2-(methylthio)pyrimidine

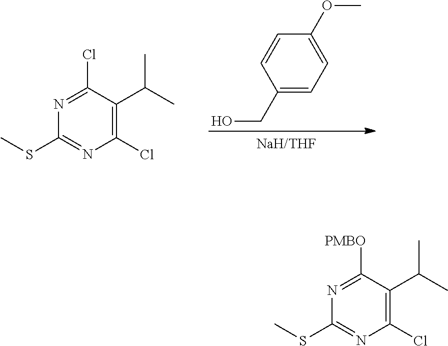

To a solution of the material from the previous step (697 mg, 2.94 mmol) and (4-methoxyphenyl)methanol (406 mg, 2.94 mmol) in THF (10 mL) under N₂ and cooled in a MeOH/H₂O/dry ice bath at −15° C. was added NaH (60% in mineral oil; 129 mg, 3.23 mmol), and the resulting mixture was allowed to warm to RT and stirred for 3 h. The reaction was neutralized with saturated NH₄Cl (1.5 mL), concentrated, diluted with H₂O (100 mL) and extracted with EtOAc (3×50 mL). The organic layers were washed with saturated NaCl, dried over MgSO₄ and concentrated. The residue was dissolved in DCM, adsorbed onto silica gel and purified via flash chromatography (0-30% (hexanes:Et₂O=8:2) in hexanes) to give the product (0.9 g, 2.66 mmol, 90% yield) as a white solid.

¹H NMR (600 MHz, DMSO-d₆) δ: 7.41 (d, J=8.3, 2H), 6.96 (d, J=8.3, 2H), 5.40 (s, 2H), 3.76 (s, 3H), 3.33-3.25 (m, 1H), 2.51 (s, 3H), 1.20 (d, J=7.2, 6H).

### Step 4: 4-chloro-5-isopropyl-6-((4-methoxybenzyl)oxy)-2-(methylsulfonyl)pyrimidine

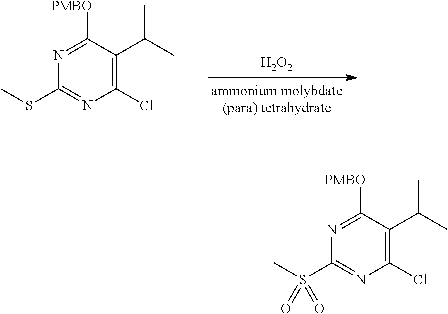

A cooled (0° C.) solution of the material from the previous step (100 mg, 0.29 mmol) in EtOH (1 mL) was treated with a cooled (0° C.) solution of H₂O₂ (0.09 mL, 0.88 mmol) and ammonium molybdate (para) hydrate (10.9 mg, 8.85 μmol), and the resulting mixture was stirred at RT overnight. An additional 3 eq of H₂O₂ (0.090 mL, 0.88 mmol) was added and the reaction stirred a further 18 h, by which time the reaction was complete. After evaporation of volatiles the mixture was dissolved in DCM and washed with water, and the organic phase was concentrated under reduced pressure. The residue was purified via silica gel chromatography (0-30% EtOAc in hexanes to give the product (100 mg, 0.27 mmol, 91% yield) as a white solid.

MS (ES⁺) C₁₆H₁₉ClN₂O₄S requires: 370, found: 393 [M+Na]⁺.

### Step 5: 4-chloro-2-(1H-imidazol-1-yl)-5-isopropyl-6-((4-methoxybenzyl)oxy)pyrimidine

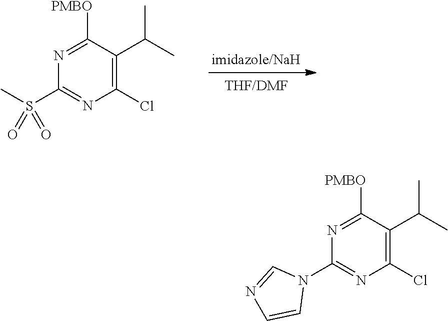

To a −78° C. cooled solution of the material from the previous step in THF (1 mL) was added dropwise a solution of imidazole (9.18 mg, 0.135 mmol) (previously treated with NaH (60% in mineral oil; 5.93 mg, 0.148 mmol) and left under stirring for 3 min and then cooled down) in DMF (1 mL) and the resulting mixture was stirred whilst allowing to warm to RT over 1 h. The mixture was quenched with saturated NH₄Cl and then taken up in DCM. The organic phase was directly purified via silica gel chromatography (0-50% EtOAc in hexanes) to give the product (26 mg, 0.072 mmol, 53% yield) as a white solid.

MS (ES⁺) C₁₈H₁₉ClN₄O₂ requires: 358, found: 359 [M+H]⁺.

### Step 6: 2-(1H-imidazol-1-yl)-5-isopropyl-4-(1-isopropyl-1H-pyrazol-4-yl)-6-((4-methoxybenzyl)-oxy)pyrimidine

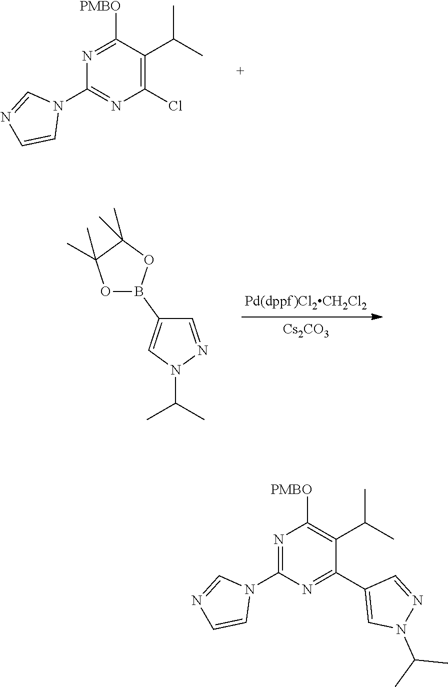

To a 4 mL vial equipped with a stir bar, the material from the previous step (100 mg, 0.279 mmol), 1-isopropyl-4-(4,4,5,5-tetramethyl-1,3,2-dioxaborolan-2-yl)-1H-pyrazole (79 mg, 0.334 mmol), Cs₂CO₃ (227 mg, 0.697 mmol), and PdCl₂(dppf)-CH₂Cl₂ adduct (22.76 mg, 0.028 mmol) were added followed by dioxane (2.8 mL) and H₂O (25 μl). The solution was degassed for 5 min, then the reaction mixture was heated to 90° C. and allowed to stir overnight. The mixture was diluted with EtOAc, 1M HCl was added, and the layers were separated. The aqueous phase was extracted with EtOAc (3×), and the combined organic layers were washed with sat. NaCl, dried over Na₂SO₄, and concentrated under reduced pressure. The residue was purified via silica gel chromatography (20-80% EtOAc in hexanes) to give the product (70 mg, 0.162 mmol, 58% yield) as an orange amorphous material.

MS (ES⁺) C₂₄H₂₈N₆O₂ requires: 432, found: 433 [M+H]⁺.

### Step 7: 2-(1H-imidazol-1-yl)-5-isopropyl-6-(1-isopropyl-1H-pyrazol-4-yl)pyrimidin-4-ol (1)

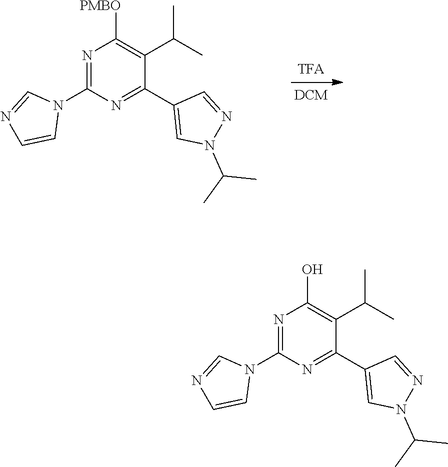

The material from the previous step (70 mg, 0.162 mmol) was treated with DCM (1 mL) and TFA (0.5 mL) and stirred for 3 h. The volatiles were removed under reduced pressure. The residue was purified by mass-triggered preparative HPLC (Mobile phase: A=0.1% TFA/H₂O, B=0.1% TFA/MeCN; Gradient: B=20-50%; 20 min; Column: C18) to give the product 1 as a white solid (TFA salt).

MS (ES⁺) C₁₆H₂₀N₆O requires: 312, found: 313 [M+H]⁺.

¹H NMR (600 MHz, DMSO-d₆) δ: 12.86 (brs, 1H), 9.18 (brs, 1H), 8.35 (s, 1H), 8.09 (s, 1H), 7.96 (s, 1H), 7.49 (s, 1H), 4.66-4.58 (m, 1H), 3.47-3.41 (m, 1H), 1.48 (d, J=6.6, 6H), 1.34 (d, J=7.0, 6H).

### Example 2

### 6-Chloro-5-(propan-2-yl)-2-[2-(pyridin-2-yl)-1H-imidazol-1-yl]pyrimidin-4-ol

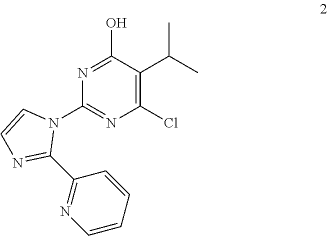 [SMILES: CC(C)c1c(O)nc(-n2ccnc2-c2ccccn2)nc1Cl]

### Step 1: 4-Chloro-5-isopropyl-6-((4-methoxybenzyl)oxy)-2-(2-(pyridin-2-yl)-1H-imidazol-1-yl)-pyrimidine

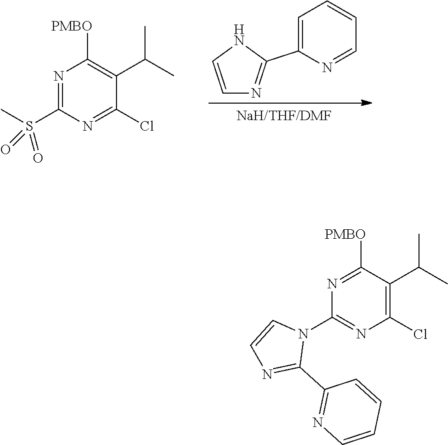

To a solution of 4-chloro-5-isopropyl-6-((4-methoxybenzyl)oxy)-2-(methylsulfonyl)-pyrimidine (Example 1, Step 4) (50 mg, 0.135 mmol) in THF (0.45 mL at −60° C.) were added dropwise a previously mixed solution of 2-(1H-imidazol-2-yl)pyridine (20.5 mg, 0.142 mmol) and NaH in 60% of mineral oil (5.9 mg, 0.148 mmol) in DMF (0.22 mL) (this solution was made at 0° C.). The resulting mixture was allowed to reach RT and stirred overnight. The mixture was quenched with 1M HCl, taken up in EtOAc and the layers were separated. The aqueous phase was extracted with EtOAc (3×) and the combined organic layers were concentrated under reduced pressure. The residue was purified via silica gel chromatography (30-100% EtOAc in hexanes) to give the product (24 mg, 0.055 mmol, 41% yield) as an orange liquid.

MS (ES⁺) C₂₃H₂₂ClN₅O₂ requires: 435, found: 436 [M+H]⁺.

### Step 2: 6-Chloro-5-isopropyl-2-(2-(pyridin-2-yl)-1H-imidazol-1-yl)pyrimidin-4-ol (2)

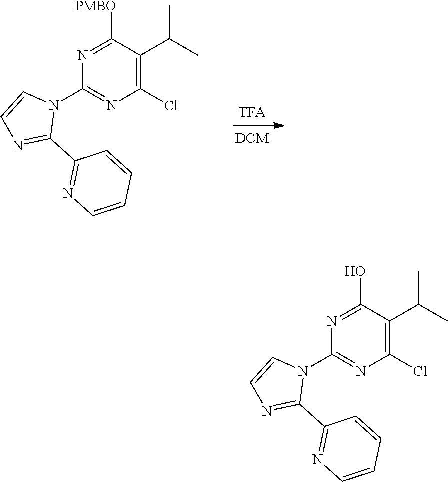

The material from the previous step was dissolved in DCM (0.5 mL). TFA (0.2 mL) was added and the mixture was stirred for 1 h. The volatiles were removed under reduced pressure and the residue was purified by mass-triggered preparative HPLC (Mobile phase: A=0.1% TFA/H₂O, B=0.1% TFA/MeCN; Gradient: B=30-70%; 12 min; Column: C18) to give the product 2 (1.1 mg, 2.7 μmol, 2% yield) as an off-white solid (TFA salt).

MS (ES⁺) C₁₅H₁₄ClN₅O requires: 315, found: 316 [M+H]⁺.

¹H NMR (600 MHz, DMSO-d₆) δ: 8.36 (s, 1H), 8.08-8.01 (m, 1H), 7.95-7.89 (m, 1H), 7.75 (s, 1H), 7.39-7.33 (m, 1H), 7.22 (s, 1H), 3.36-3.29 (m, 1H), 1.29 (d, J=7.0 Hz, 6H).

### Example 3

### 5-(Propan-2-yl)-6-[1-(propan-2-yl)-1H-pyrazol-4-yl]-2-[2-(pyridin-2-yl)-1H-imidazol-1-yl]pyrimidin-4-ol

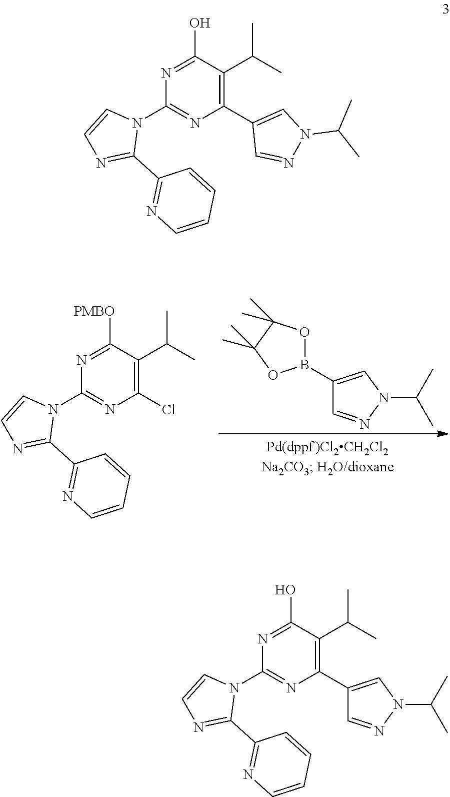

A microwave vial was charged with 4-chloro-5-isopropyl-6-((4-methoxybenzyl)oxy)-2-(2-(pyridin-2-yl)-1H-imidazol-1-yl)pyrimidine (Example 2, Step 1) (15 mg, 0.034 mmol), 1-isopropyl-4-(4,4,5,5-tetramethyl-1,3,2-dioxaborolan-2-yl)-1H-pyrazole (12.1 mg, 0.052 mmol), PdCl₂(dppf)-CH₂Cl₂ adduct (5.6 mg, 6.88 μmol) and Na₂CO₃ (7.2 mg, 0.069 mmol) in dioxane (0.15 mL) and H₂O (0.019 mL). The vial was sealed and the reaction mixture was heated to 150° C. in the microwave reactor for 2 h. Complete conversion and removal of the PMB group were both observed. The reaction mixture was directly purified via mass-triggered preparative HPLC (Mobile phase: A=0.1% TFA/H₂O, B=0.1% TFA/MeCN; Gradient: B=30-70%; 12 min; Column: C18) to give the product 3 (2.4 mg, 4.85 μmol, 14% yield) as a pale yellow amorphous material (TFA salt).

MS (ES⁺) C₂₁H₂₃N₇O requires: 389, found: 390 [M+H]⁺.

¹H NMR (600 MHz, DMSO-d₆) δ: 8.42-8.37 (m, 1H), 8.05-7.92 (m, 2H), 7.80 (s, 2H), 7.48 (brs, 1H), 7.41-7.37 (m, 1H), 7.26 (s, 1H), 4.55-4.47 (m, 1H), 3.36-3.29 (m, 1H), 1.39 (d, J=6.6, 6H), 1.34 (d, J=7.0, 6H).

### Example 4

### 5-isopropyl-6-(1-isopropyl-1H-pyrazol-4-yl)-2-(2-methyl-1H-imidazol-1-yl)pyrimidin-4-ol

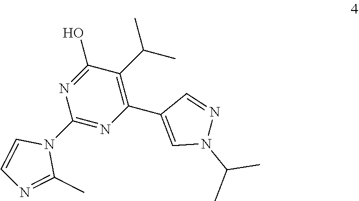 [SMILES: Cc1nccn1-c1nc(O)c(C(C)C)c(-c2cnn(C(C)C)c2)n1]

### Step 1: 5-Isopropyl-4-(1-isopropyl-1H-pyrazol-4-yl)-6-((4-methoxybenzyl)oxy)-2-(methylthio)-pyrimidine

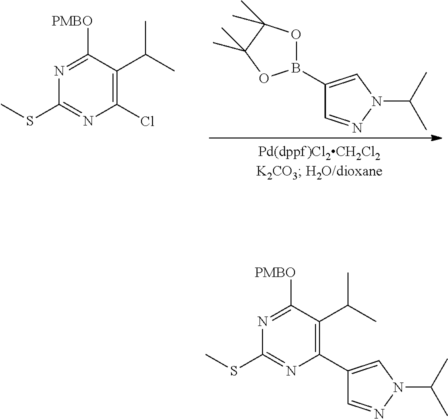

To a mixture of 4-chloro-5-isopropyl-6-((4-methoxybenzyl)oxy)-2-(methylthio)-pyrimidine (Example 1, Step 3) (70 mg, 0.21 mmol) under N₂, 1-isopropyl-4-(4,4,5,5-tetramethyl-1,3,2-dioxaborolan-2-yl)-1H-pyrazole (53 mg, 0.23 mmol) and PdCl₂(dppf)-CH₂Cl₂ adduct (17 mg, 0.021 mmol) was added dioxane (1 mL) and a 2M solution of K₂CO₃ (100 al, 0.205 mmol). The mixture was purged with N₂ and stirred at 80° C. for 2.5 h. The reaction was partitioned between DCM and H₂O, the organic layer was separated, and the aqueous layer was extracted with DCM. The organic layers were combined, washed with saturated NaCl, dried over MgSO₄, concentrated, and supported on silica gel and purified via silica gel chromatography (0-20% EtOAc in hexanes) to give 5-isopropyl-4-(1-isopropyl-1H-pyrazol-4-yl)-6-((4-methoxy-benzyl)oxy)-2-(methylthio)pyrimidine (46 mg, 0.11 mmol, 54% yield) as a colorless film on glass. MS (ES⁺) C₂₂H₂₈N₄O₂S requires: 412, found: 413 [M+H]⁺.

### Step 2: 5-Isopropyl-4-(1-isopropyl-1H-pyrazol-4-yl)-6-((4-methoxybenzyl)oxy)-2-(methylsulfonyl)pyrimidine

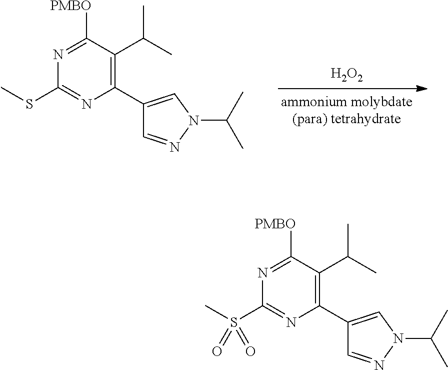

To a vial containing ammonium molybdate tetrahydrate (7 mg, 5.7 μmol) dissolved in hydrogen peroxide (32 μl, 0.31 mmol) was added a cooled solution of the material from the previous step (43 mg, 0.10 mmol) in EtOH (700 μl). The reaction was stirred in an ice bath and allowed to warm to RT and stirred for 16 h. Additional EtOH (1 mL) was added, the reaction was cooled in an ice bath, and additional ammonium molybdate tetrahydrate (7 mg, 5.7 μmol) dissolved in hydrogen peroxide (32 μl, 0.31 mmol) was added. The reaction was allowed to warm to RT and stirred for 6 h. The reaction was diluted with water and extracted with DCM (4×). The organic layers were combined, washed with saturated NaCl, dried over MgSO₄, concentrated, adsorbed onto silica gel, and purified via flash chromatography (0-60% EtOAc in hexanes) to give the product (38 mg, 0.086 mmol, 82% yield) as a white opaque waxy solid.

MS (ES⁺) C₂₂H₂₈N₄O₄S requires: 444, found: 445 [M+H]⁺.

### Step 3: 5-Isopropyl-6-(1-isopropyl-1H-pyrazol-4-yl)-2-(2-methyl-1H-imidazol-1-yl)pyrimidin-4-ol (4)

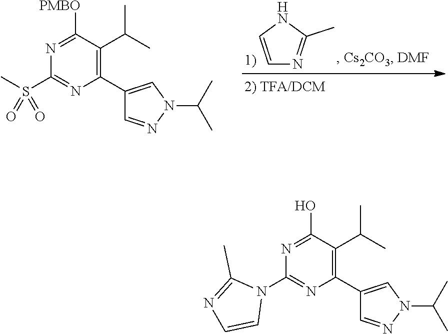

To a vial containing 2-methyl-imidazole (12 mg, 0.14 mmol) and Cs₂CO₃ (46 mg, 0.14 mmol) was added a solution of the material from the previous step (21 mg, 0.047 mmol) dissolved in DMF (472 al) and the resulting mixture was stirred at 60° C. for 12 h. The reaction was diluted with DCM, filtered through a cotton plug, rinsed with DCM, and concentrated. The residue was dissolved in DCM (0.4 mL) and TFA (0.2 mL) and stirred at room temperature for 3 h. The reaction was concentrated, dissolved in DMSO:MeOH (0.3:0.3 mL) and filtered. The solid was rinsed once with DMSO:MeOH (0.2:0.2 mL). The filtrate was purified by mass-triggered preparative HPLC (Mobile phase: A=0.1% TFA/H₂O, B=0.1% TFA/MeCN; Gradient: B=10-40%; 12 min; Column: C18) to give the product 4 (13 mg, 0.029 mmol, 61% yield) as a white solid (TFA salt).

MS (ES⁺) C₁₇H₂₂N₆O requires: 326, found: 327 [M+H]⁺.

¹H NMR (600 MHz, Methanol-d₄) δ: 8.27 (d, J=1.9, 1H), 8.17 (s, 1H), 7.93 (s, 1H), 7.54 (d, J=1.9, 1H), 4.65 (spt, J=6.7, 1H), 3.53 (spt, J=6.9, 1H), 3.09 (s, 3H), 1.56 (d, J=6.8, 6H), 1.43 (d, J=6.8, 6H).

### Example 5

### 2-(4-(6-Hydroxy-2-(1H-imidazol-1-yl)-5-isopropylpyrimidin-4-yl)-1H-pyrazol-1-yl)acetic acid

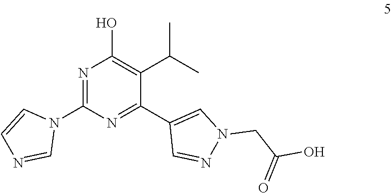 [SMILES: CC(C)c1c(O)nc(-n2ccnc2)nc1-c1cnn(CC(=O)O)c1]

### Step 1: 2-(4-(2-(1H-imidazol-1-yl)-5-isopropyl-6-((4-methoxybenzyl)oxy)pyrimidin-4-yl)-1H-pyrazol-1-yl)acetic acid

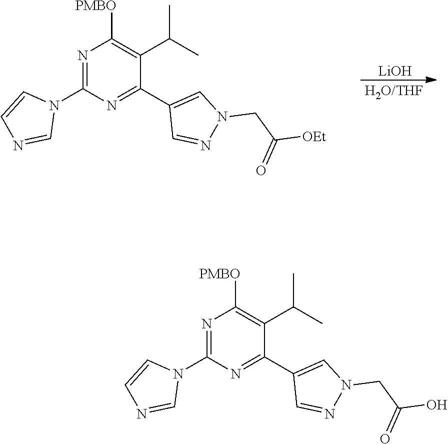

To a stirred solution of ethyl 2-(4-(2-(1H-imidazol-1-yl)-5-isopropyl-6-((4-methoxy-benzyl)oxy)pyrimidin-4-yl)-1H-pyrazol-1-yl)acetate (93.5 mg, 0.196 mmol) (prepared by the method of Example 1 using 1-(ethoxycarbonylmethyl)-1H-pyrazole-4-boronic acid pinacol ester), in THF (1.5 mL) and H₂O (0.4 mL), was added 2 M aqueous LiOH (0.108 mL, 0.216 mmol) and the reaction mixture was heated to 40° C. and allowed to stir for 15 min. The volatiles were removed under reduced pressure. Theoretical yield was assumed and the crude material was taken to the next step without purification. MS (ES⁺) C₂₃H₂₄N₆O₄ requires: 448, found: 449 [M+H]⁺.

### Step 2: 2-(4-(6-Hydroxy-2-(1H-imidazol-1-yl)-5-isopropylpyrimidin-4-yl)-1H-pyrazol-1-yl)acetic acid (5)

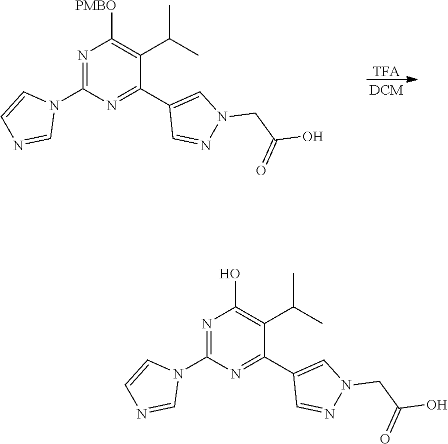

To a stirred solution of the product from the previous step (96 mg, 0.214 mmol) in DCM (1 mL), TFA (0.6 mL) was added and the mixture was allowed to stir at RT for 18 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 12 min; Column: C18) to give the product 5 (55.3 mg, 0.12 mmol, 58% yield) as a white amorphous solid (TFA salt).

MS (ES⁺) C₁₅H₁₆N₆O₃ requires: 328, found: 329 [M+H]⁺.

¹H NMR (600 MHz, DMSO-d₆) δ: 13.20 (brs, 1H), 12.96 (brs, 1H), 9.24 (brs, 1H), 8.38 (s, 1H), 8.10 (s, 1H), 8.06 (s, 1H), 7.54 (brs, 1H), 5.06 (s, 2H), 3.45-3.40 (m, 1H), 1.34 (d, J=7.2, 6H).

### Example 6

### 2-(4-(6-Hydroxy-2-(1H-imidazol-1-yl)-5-isopropylpyrimidin-4-yl)-1H-pyrazol-1-yl)-N-(2-(pyrrolidin-1-yl)ethyl)acetamide

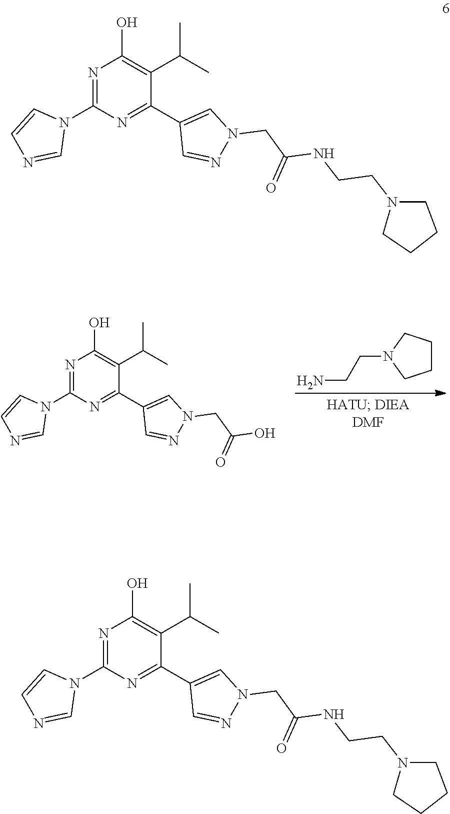 [SMILES: CC(C)c1c(O)nc(-n2ccnc2)nc1-c1cnn(CC(=O)NCCN2CCCC2)c1 ; CC(C)c1c(O)nc(-n2ccnc2)nc1-c1cnn(CC(=O)O)c1 ; NCCN1CCCC1 ; CC(C)c1c(O)nc(-n2ccnc2)nc1-c1cnn(CC(=O)NCCN2CCCC2)c1]

To a stirred solution of 2-(4-(6-hydroxy-2-(1H-imidazo 1-yl)-5-isopropylpyrimidin-4-yl)-1H-pyrazol-1-yl)acetic acid (Example 5, Step 2) (10 mg, 0.030 mmol) in DMF (0.3 mL) were added 2-(pyrrolidin-1-yl)ethanamine (3.8 mg, 0.034 mmol), DIEA (10.6 μl, 0.061 mmol), and HATU (17.3 mg, 0.046 mmol) and the reaction mixture was allowed to stir at RT for 16 h. The volatiles were removed under reduced pressure. The residue was purified by mass-triggered preparative HPLC (Mobile phase: A=0.1% TFA/H₂O, B=0.1% TFA/MeCN; Gradient: B=10-30%; 20 min; Column: C18) to give the product 6 (4.1 mg, 6.2 μmol, 20% yield) as a white solid (bis-TFA salt).

MS (ES⁺) C₂₁H₂₈N₈O₂ requires: 424, found: 425 [M+H]⁺.

¹H NMR (600 MHz, DMSO-d₆) δ: 13.01 (brs, 1H), 9.68 (brs, 1H), 9.15 (brs, 1H), 8.51 (t, J=5.7, 1H), 8.34 (s, 1H), 8.08 (s, 1H), 8.06 (s, 1H), 7.49 (brs, 1H), 4.94 (s, 2H), 3.62-3.54 (m, 2H), 3.49-3.40 (m, 3H), 3.27-3.22 (m, 2H), 3.08-2.99 (m, 2H), 2.05-1.97 (m, 2H), 1.90-1.81 (m, 2H), 1.35 (d, J=6.8, 6H).

### Example 7

### 2-(4-(6-Hydroxy-2-(1H-imidazol-1-yl)-5-isopropylpyrimidin-4-yl)-1H-pyrazol-1-yl)-acetamide

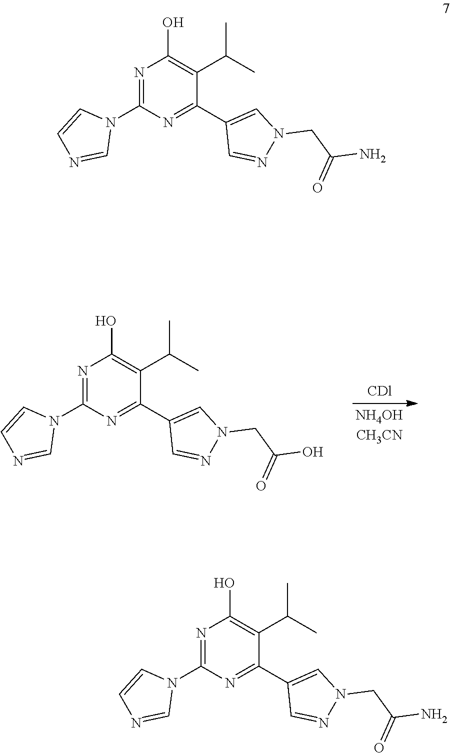 [SMILES: CC(C)c1c(O)nc(-n2ccnc2)nc1-c1cnn(CC(N)=O)c1 ; CC(C)c1c(O)nc(-n2ccnc2)nc1-c1cnn(CC(=O)O)c1 ; CC(C)c1c(O)nc(-n2ccnc2)nc1-c1cnn(CC(N)=O)c1]

To a stirred solution of 2-(4-(6-hydroxy-2-(1H-imidazol-1-yl)-5-isopropylpyrimidin-4-yl)-1H-pyrazol-1-yl)acetic acid (Example 5, Step 2) (10 mg, 0.030 mmol) in MeCN (0.15 mL) was added CDI (5.4 mg, 0.034 mmol) and the mixture was allowed to stir at RT for 2 h. Then aqueous NH₄OH (23.7 μl, 0.609 mmol; 29% w.t.) was added upon which the reaction mixture turned dark green. The reaction mixture was allowed to stir for a total of 4 h, then diluted with EtOAc (10 mL) and washed with sat. NaHCO₃ (3×10 mL). The layers were separated, and the organic layer was washed with sat. NaCl (10 mL), dried over Na₂SO₄, and concentrated under reduced pressure. The residue was purified by mass-triggered preparative HPLC (Mobile phase: A=0.1% TFA/H₂O, B=0.1% TFA/MeCN; Gradient: B=10-50%; 12 min; Column: C18) to give the product 7 (0.38 mg, 0.86 μmol, 2.8% yield) as a TFA salt.

MS (ES⁺) C₁₅H₁₇N₇O₂ requires: 327, found: 328 [M+H]⁺.

¹H NMR (600 MHz, Methanol-d₄) δ: 9.53 (s, 1H), 8.30 (s, 1H), 8.27 (s, 1H), 8.04 (s, 1H), 7.61 (s, 1H), 4.98 (s, 2H), 3.60-3.52 (m, 1H), 1.42 (d, J=7.2, 6H).

### Example 8

### N-(2-(dimethylamino)ethyl)-5-(6-hydroxy-2-(1H-imidazol-1-yl)-5-isopropylpyrimidin-4-yl)-picolinamide

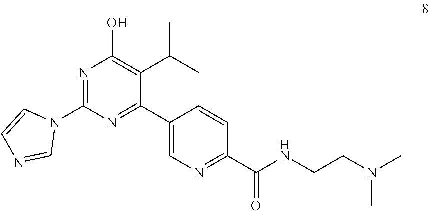 [SMILES: CC(C)c1c(O)nc(-n2ccnc2)nc1-c1ccc(C(=O)NCCN(C)C)nc1]

### Step 1: 5-(2-(1H-imidazol-1-yl)-5-isopropyl-6-((4-methoxybenzyl)oxy)pyrimidin-4-yl)picolinic acid

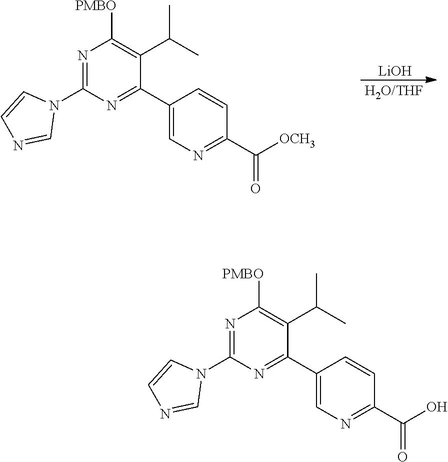

To a stirred solution of methyl 5-(2-(1H-imidazol-1-yl)-5-isopropyl-6-((4-methoxy-benzyl)oxy)pyrimidin-4-yl)picolinate (109 mg, 0.237 mmol) (prepared by the method of Example 1 from 2-methoxycarbonyl-3-fluoropyridine-5-boronic acid pinacol ester) in THF (1.9 mL) and H₂O (0.47 mL) was added 2M aqueous LiOH (0.131 mL, 0.261 mmol) and the reaction mixture was heated to 40° C. After 1 h, the volatiles were removed under reduced pressure. The reaction mixture was diluted with DCM and filtered, and the filtrate was concentrated under reduced pressure to give the product (72 mg, 0.16 mmol, 67% yield) as a brown solid. The material was taken forward without purification.

MS (ES⁺) C₂₄H₂₃N₅O₄ requires: 445, found: 446 [M+H]⁺.

### Step 2: 5-(6-hydroxy-2-(1H-imidazol-1-yl)-5-isopropylpyrimidin-4-yl)picolinic acid

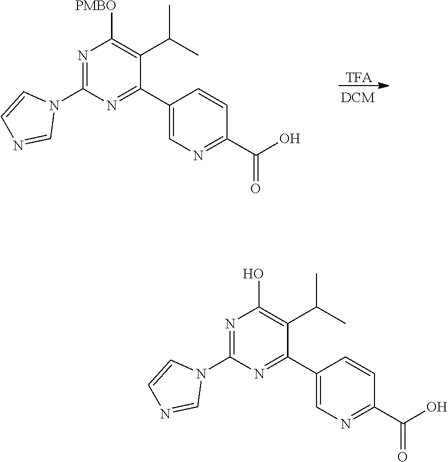

To a stirred solution of the material from the previous step (72 mg, 0.16 mmol) in DCM (0.8 mL) was added TFA (0.4 mL) and the mixture was allowed to stir at RT for 6 days. The volatiles were removed under reduced pressure. The residue was purified by mass-triggered preparative HPLC (Mobile phase: A=0.1% TFA/H₂O, B=0.1% TFA/MeCN; Gradient: B=10-50%; 12 min; Column: C18) to give the product (33 mg, 0.06 mmol, 37% yield) as a white solid (bis-TFA salt).

MS (ES⁺) C₁₆H₁₅N₅O₃ requires: 325, found: 326 [M+H]⁺.

¹H NMR (600 MHz, DMSO-d₆) δ: 13.43 (s, 1H), 9.00 (brs, 1H), 8.85 (d, J=2.3, 1H), 8.20-8.19 (m, 1H), 8.15-8.14 (m, 1H), 8.00 (brs, 1H), 7.42 (brs, 1H), 2.96-2.91 (m, 1H), 1.29 (d, J=6.8, 6H).

### Step 3: N-(2-(dimethylamino)ethyl)-5-(6-hydroxy-2-(1H-imidazol-1-yl)-5-isopropylpyrimidin-4-yl)picolinamide (8)

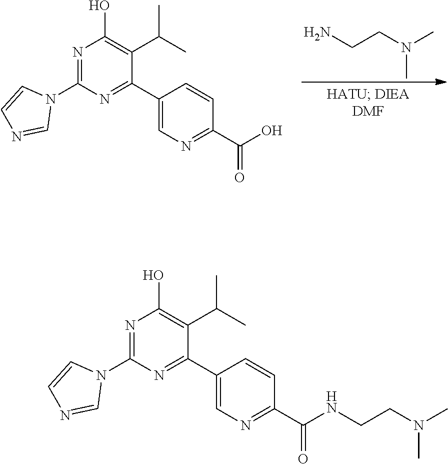

To a stirred solution of the material from the previous step (8.1 mg, 0.025 mmol) in DMF (0.25 mL) were added N,N-dimethylethane-1,2-diamine (2.4 mg, 0.027 mmol), HATU (14.20 mg, 0.037 mmol), and DIEA (8.70 al, 0.050 mmol). The reaction mixture was allowed to stir at RT for 18 h. The volatiles were removed under reduced pressure. The residue was purified by mass-triggered preparative HPLC (Mobile phase: A=0.1% TFA/H₂O, B=0.1% TFA/MeCN; Gradient: B=10-30%; 12 min; Column: C18) to give the product 8 (6.7 mg, 0.017 mmol, 68% yield) as a white solid.

MS (ES⁺) C₂₀H₂₅N₇O₂ requires: 395, found: 396 [M+H]⁺.

¹H NMR (600 MHz, DMSO-d₆) δ: 13.48 (brs, 1H), 9.38 (brs, 1H), 9.22 (t, J=6.2, 1H), 8.88 (brs, 1H), 8.81-8.79 (m, 1H), 8.24-8.16 (m, 2H), 7.96 (s, 1H), 7.37 (s, 1H), 3.68-3.71 (m, 2H), 3.34-3.31 (m, 2H), 2.93-2.88 (m, 1H), 2.86 (d, J=3.8, 6H), 1.28-1.29 (d, J=6.8, 6H).

### Example 9

### 5-(6-hydroxy-2-(1H-imidazol-1-yl)-5-isopropylpyrimidin-4-yl)picolinamide

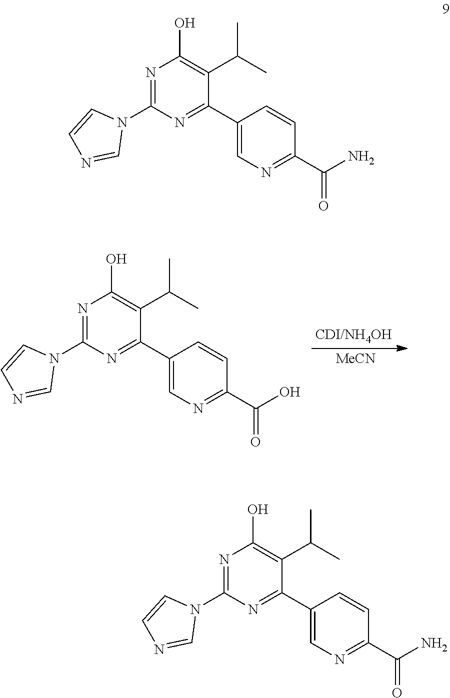 [SMILES: "MeCN" ; CC(C)c1c(O)nc(-n2ccnc2)nc1-c1ccc(C(N)=O)nc1 ; CC(C)c1c(O)nc(-n2ccnc2)nc1-c1ccc(C(=O)O)nc1 ; CC(C)c1c(O)nc(-n2ccnc2)nc1-c1ccc(C(N)=O)nc1]

To a stirred solution of 5-(6-hydroxy-2-(1H-imidazol-1-yl)-5-isopropylpyrimidin-4-yl)picolinic acid (Example 8, Step 2) (10 mg, 0.031 mmol) in MeCN (0.3 mL) was added CDI (4.98 mg, 0.031 mmol), and the mixture was allowed to stir at RT for 1 h. Aqueous NH₄OH (23.94 al, 0.615 mmol; 29% w.t.) was then added and the reaction mixture was allowed to stir for 30 min. The volatiles were removed under reduced pressure. The residue was purified by mass-triggered preparative HPLC (Mobile phase: A=0.1% TFA/H₂O, B=0.1% TFA/MeCN; Gradient: B=10-30%; 12 min; Column: C18) to give the product 9 (6 mg, 0.011 mmol, 36% yield) as a white solid (bis-TFA salt).

MS (ES⁺) C₁₆H₁₆N₆O₂ requires: 324, found: 325 [M+H]⁺.

¹H NMR (600 MHz, DMSO-d₆) δ: 13.38 (brs, 1H), 8.92 (brs, 1H), 8.78 (s, 1H), 8.21-8.14 (m, 3H), 7.98 (brs, 1H), 7.77 (brs, 1H), 7.38 (brs, 1H), 2.95-2.93 (m, 1H), 1.29 (d, J=6.8, 6H).

### Example 10a/b

### 2-(1H-imidazol-1-yl)-5-(propan-2-yl)-6-[2-(pyridin-3-yl)ethynyl]pyrimidin-4-ol

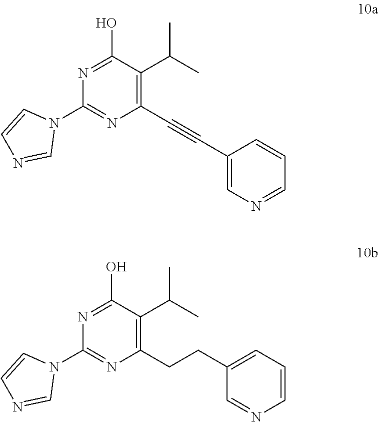 [SMILES: CC(C)c1c(O)nc(-n2ccnc2)nc1C#Cc1cccnc1 ; CC(C)c1c(O)nc(-n2ccnc2)nc1CCc1cccnc1]

### Step 1: 2-(1H-imidazol-1-yl)-5-isopropyl-4-((4-methoxybenzyl)oxy)-6-(pyridin-3-ylethynyl)-pyrimidine

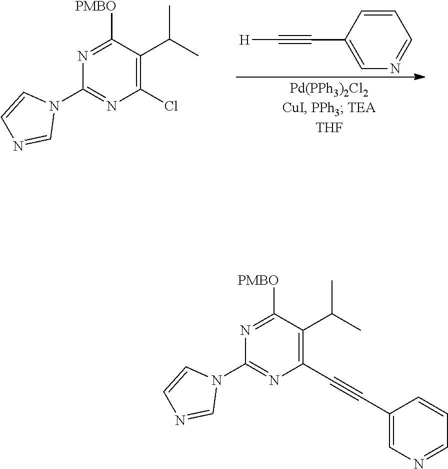

To a solution of 4-chloro-2-(1H-imidazol-1-yl)-5-isopropyl-6-((4-methoxybenzyl)-oxy)pyrimidine (75 mg, 0.209 mmol) (Example 1, Step 5) in THF (1 mL) were added 3-ethynyl-pyridine (25.9 mg, 0.251 mmol), CuI (1.194 mg, 6.27 μmol), PPh₃ (4.39 mg, 0.017 mmol), Pd(PPh₃)₂Cl₂ (4.40 mg, 6.27 μmol) and TEA (87 al, 0.627 mmol), and the resulting mixture was degassed for 5 min then stirred at 70° C. overnight. The volatiles were removed under reduced pressure and the residue was purified via silica gel chromatography (20-100% EtOAc in hexanes to give the product (27 mg, 0.063 mmol, 30% yield) as a yellow amorphous material.

MS (ES⁺) C₂₅H₂₃N₅O₂ requires: 425, found: 426 [M+H]⁺.

### Step 2: 2-(1H-imidazol-1-yl)-5-(propan-2-yl)-6-[2-(pyridin-3-yl)ethyl]pyrimidin-4-ol (10a)

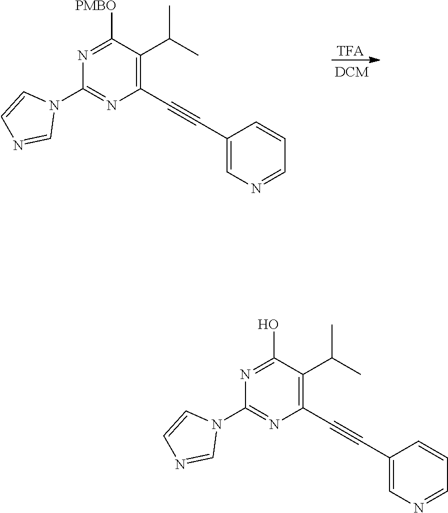

An aliquot of the reaction mixture from Step 1 was concentrated and the residue was taken up in DCM (0.5 mL). TFA (0.25 mL) was added and the mixture was stirred at RT for 1 h. The volatiles were removed under reduced pressure and the residue was purified by mass-triggered preparative HPLC (Mobile phase: A=0.1% TFA/H₂O, B=0.1% TFA/MeCN; Gradient: B=10-40%; 20 min; Column: C18) to give the product 10a (2.5 mg, 4.65 μmol, 2% yield) as a white solid (bis-TFA salt).

MS (ES⁺) C₁₇H₁₅N₅O requires: 305, found: 306 [M+H]⁺.

¹H NMR (600 MHz, DMSO-d₆) δ: 9.08 (brs, 1H), 8.86 (d, J=1.6, 1H), 8.70 (dd, J=4.8, 1.5, 1H), 8.12-8.09 (m, 1H), 8.00 (brs, 1H), 7.58-7.52 (m, 1H), 7.47 (brs, 1H), 5.59-5.52 (m, 1H), 1.35 (d, J=6.9, 6H).

### Step 3: 2-(1H-imidazol-1-yl)-5-(propan-2-yl)-6-[2-(pyridin-3-yl)ethyl]pyrimidin-4-ol (10b)

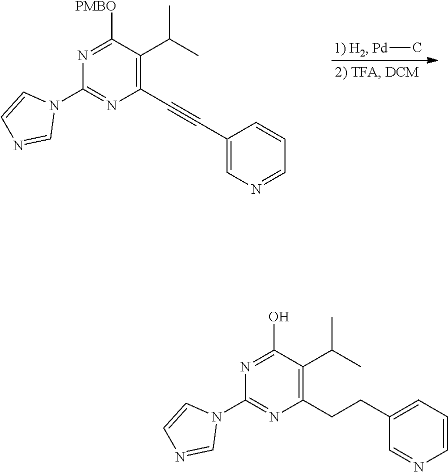

A reaction vessel was charged with 2-(1H-imidazol-1-yl)-5-isopropyl-4-((4-methoxy-benzyl)oxy)-6-(pyridin-3-ylethynyl)pyrimidine (from Step 1) (20 mg, 0.047 mmol), 10% Pd—C (5.0 mg, 4.70 μmol) and EtOAc (1.5 mL). The solution was degassed with N₂ and purged with H₂. The reaction mixture was stirred under an atmosphere of H₂ at 40 psi overnight. The reaction mixture was purged with N₂, filtered through Celite, and concentrated under reduced pressure. To the residue was added DCM (0.5 mL) and TFA (0.25 mL) and the mixture was stirred for 1 h at RT. The solution was concentrated and the residue was purified by mass-triggered preparative HPLC (Mobile phase: A=0.1% TFA/H₂O, B=0.1% TFA/MeCN; Gradient: B=0-40%; 20 min; Column: C18) to give the product 10b (5 mg, 0.009 mmol, 20% yield) as a bis-TFA salt.

MS (ES⁺) C₁₇H₁₉N₅O requires: 309, found: 310 [M+H]⁺.

¹H NMR (600 MHz, DMSO-d₆) δ: 12.84 (brs, 1H), 9.00 (s, 1H), 8.68 (s, 1H), 8.59 (d, J=5.0, 1H), 8.13 (s, 1H), 7.97 (s, 1H), 7.67-7.63 (m, 1H), 7.44 (s, 1H), 3.21-3.13 (m, 5H), 1.17 (d, J=6.9, 6H).

### Example 11

### 2-(1H-imidazol-1-yl)-6-[2-(1-methylpiperidin-4-yl)ethoxy]-5-(propan-2-yl)pyrimidin-4-ol

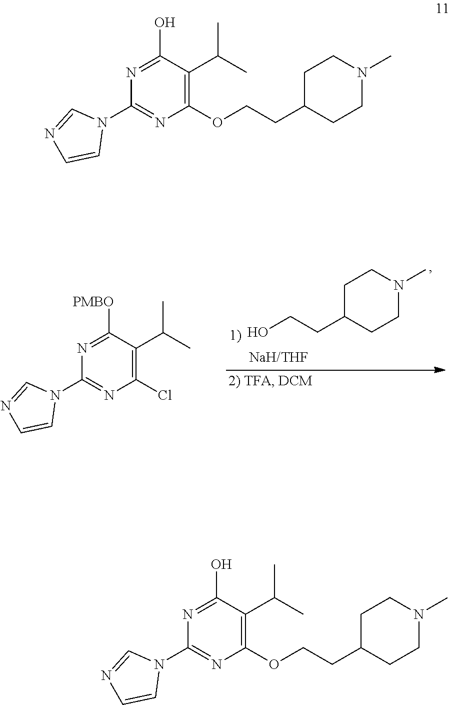

To a solution of 4-chloro-2-(1H-imidazol-1-yl)-5-isopropyl-6-((4-methoxybenzyl)-oxy)pyrimidine (20 mg, 0.056 mmol) (Example 1, Step 5) in THF (0.5 mL) at −30° C. was added a pre-prepared solution of [2-(1-methylpiperidin-4-yl)ethanol (12 mg, 0.084 mmol) and NaH in 60% mineral oil (3.34 mg, 0.084 mmol) in THF (0.5 mL), dropwise, and the mixture was allowed to reach RT and then heated at 50° C. overnight. The mixture was quenched by addition of 1M HCl, then diluted with EtOAc and the layers were separated. The aqueous phase was extracted with EtOAc (3×) and combined organic extracts were concentrated. The residue was dissolved in DCM (0.5 mL), TFA (0.2 mL) was added and the mixture was stirred for 1 h at RT. The volatiles were removed under reduced pressure and the residue was purified by mass-triggered preparative HPLC (Mobile phase: A=0.1% TFA/H₂O, B=0.1% TFA/MeCN; Gradient: B=10-40%; 20 min; Column: C18) to give the product 11 (1.7 mg, 2.93 μmol, 5% yield) as a bis-TFA salt.

MS (ES⁺) C₁₈H₂₇N₅O₂ requires: 345, found: 346 [M+H]⁺.

¹H NMR (600 MHz, DMSO-d₆+5 ul of TFA) δ: 9.73 (s, 1H), 9.27 (brs, 1H), 8.20-8.18 (m, 1H), 7.88-7.85 (m, 1H), 4.51 (t, J=6.3, 2H), 3.46-3.40 (m, 2H), 3.33-3.26 (m, 1H), 2.96-2.87 (m, 2H), 2.76 (d, J=4.8, 3H), 1.98-1.91 (m, 2H), 1.77-1.71 (m, 2H), 1.75-1.69 (m, 1H), 1.45-1.34 (m, 2H), 1.23 (d, J=7.0, 6H).

### Example 12

### 2-(1H-Imidazol-1-yl)-5-isopropyl-6-methoxypyrimidin-4-ol

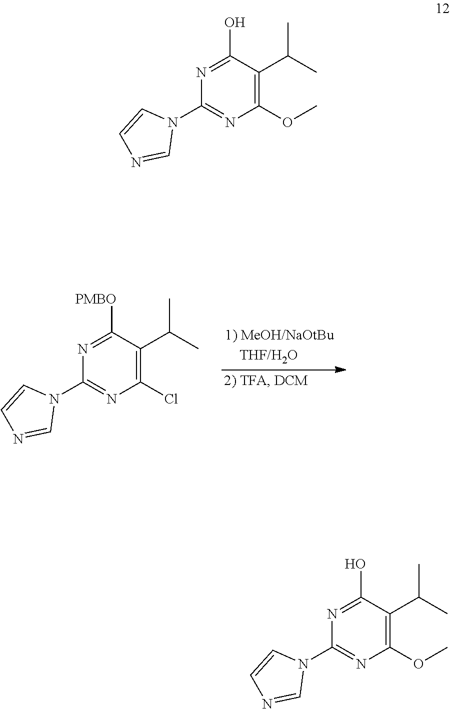

To a solution of 4-chloro-2-(1H-imidazol-1-yl)-5-isopropyl-6-((4-methoxybenzyl)-oxy)pyrimidine (Example 1, Step 5) (20 mg, 0.056 mmol) in THF (0.5 mL) and H₂O (0.5 mL) was added NaOtBu (0.056 mL, 0.167 mmol), and the resulting mixture was stirred at RT overnight. MeOH (0.2 mL) was added to make the solution homogeneous. The volatiles were removed under reduced pressure. The residue was dissolved in DCM (0.5 mL), TFA (0.2 mL) was added and the mixture was stirred for 1 h at RT, concentrated and purified by mass-triggered preparative HPLC (Mobile phase: A=0.1% TFA/H₂O, B=0.1% TFA/MeCN; Gradient: B=10-40%; 20 min; Column: C18) to give the product 12 (1.2 mg, 3.45 μmol, 6% yield) as a white solid TFA salt.

MS (ES⁺) C₁₁H₁₄N₄O₂ requires: 234, found: 235 [M+H]+.

¹H NMR (600 MHz, DMSO-d₆) δ: 12.39 (brs, 1H), 9.05 (s, 1H), 7.99 (s, 1H), 7.45 (s, 1H), 4.01 (s, 3H), 3.3-3.21 (m, 1H), 1.21 (d, J=7.0, 6H).

### Example 13

### 2-(1H-imidazol-1-yl)-5-(propan-2-yl)-6-{2-[1-(4,4,4-trifluorobutyl)piperidin-4-yl]ethoxy}pyrimidin-4-ol

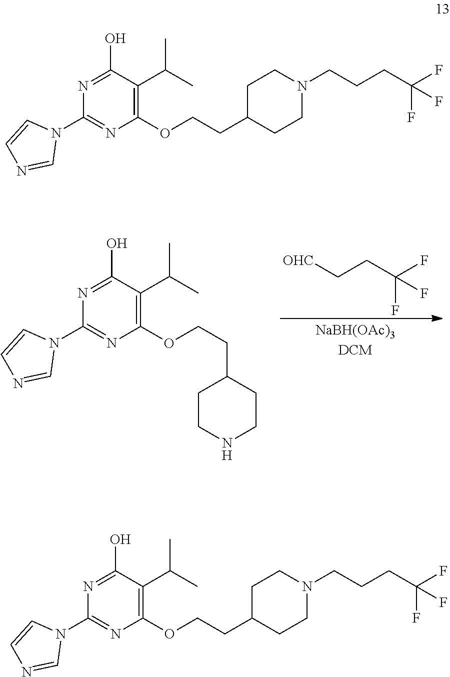 [SMILES: CC(C)c1c(O)nc(-n2ccnc2)nc1OCCC1CCN(CCCC(F)(F)F)CC1 ; CC(C)c1c(O)nc(-n2ccnc2)nc1OCCC1CCNCC1 ; O=CCCC(F)(F)F ; CC(C)c1c(O)nc(-n2ccnc2)nc1OCCC1CCN(CCCC(F)(F)F)CC1]

To a solution of 2-(1H-imidazol-1-yl)-5-isopropyl-6-(2-(piperidin-4-yl)ethoxy)-pyrimidin-4-ol (15 mg, 0.045 mmol) (prepared by the method of Example 11 from 1-(tert-butoxycarbonyl)-4-(2-hydroxyethyl)piperidine and following deprotection with DCM and TFA) in DCM (0.5 mL) were added 4,4,4-trifluorobutanal (8.56 mg, 0.068 mmol) and NaBH(OAc)₃ (14.3 mg, 0.068 mmol), and the resulting mixture was stirred at RT overnight. The mixture was concentrated and the residue was purified by mass-triggered preparative HPLC (Mobile phase: A=0.1% TFA/H₂O, B=0.1% TFA/MeCN; Gradient: B=20-50%; 16 min; Column: C18) to give the product 13 as a bis-TFA salt.

MS (ES⁺) C₂₁H₃₀F₃N₅O₂ requires: 441, found: 442 [M+H]⁺.

¹H NMR (600 MHz, DMSO-d₆) δ: 12.27 (brs, 1H), 9.08 (s, 1H), 8.65 (s, 1H), 7.86 (s, 1H), 7.22 (s, 1H), 4.51-4.44 (m, 2H), 3.28-3.21 (m, 2H), 3.13-3.06 (m, 2H), 2.95-2.87 (m, 2H), 2.41-2.3 (m, 3H), 1.99-1.82 (m, 4H), 1.76-1.69 (m, 2H), 1.47-1.36 (m, 2H), 1.21 (d, J=7.0, 6H).

### Example 14

### 3-hydroxy-1-[4-(2-{[6-hydroxy-2-(1H-imidazol-1-yl)-5-(propan-2-yl)pyrimidin-4-yl]oxy}-ethyl)piperidin-1-yl]propan-1-one

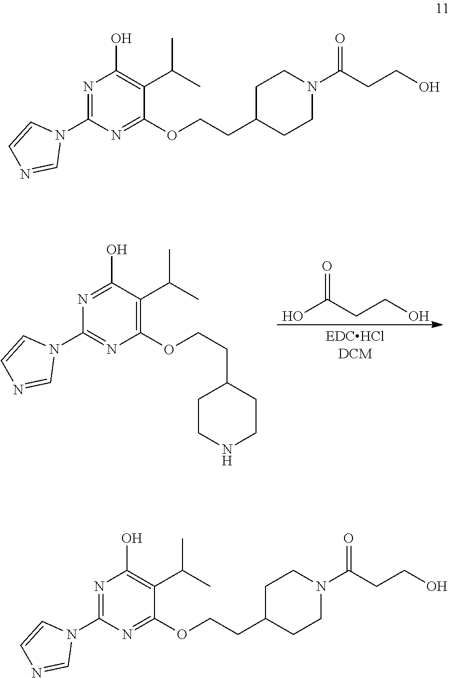 [SMILES: CC(C)c1c(O)nc(-n2ccnc2)nc1OCCC1CCN(C(=O)CCO)CC1 ; CC(C)c1c(O)nc(-n2ccnc2)nc1OCCC1CCNCC1 ; O=C(O)CCO ; CC(C)c1c(O)nc(-n2ccnc2)nc1OCCC1CCN(C(=O)CCO)CC1]

To a solution of 2-(1H-imidazol-1-yl)-5-isopropyl-6-(2-(piperidin-4-yl)ethoxy)-pyrimidin-4-ol (15 mg, 0.045 mmol) (prepared by the method of Example 11 from 1-(tert-butoxycarbonyl)-4-(2-hydroxyethyl)piperidine, followed by deprotection with DCM and TFA) in DCM (0.5 mL) were added 3-hydroxypropanoic acid (4.89 mg, 0.054 mmol), DIEA (0.024 mL, 0.136 mmol) and 1-ethyl-3-(3-dimethylaminopropyl)carbodiimide hydrochloride (10.4 mg, 0.054 mmol) and the resulting mixture was stirred at RT overnight. The mixture was neutralized with 1M HCl and concentrated. The residue was purified by mass-triggered preparative HPLC (Mobile phase: A=0.1% TFA/H₂O, B=0.1% TFA/MeCN; Gradient: B=20-50%; 20 min; Column: C18) to give the product 14 (2.0 mg, 3.86 μmol, 9% yield) as a TFA salt.

MS (ES⁺) C₂₀H₂₉N₅O₄ requires: 403, found: 404 [M+H]⁺.

¹H NMR (600 MHz, DMSO-d₆) δ: 12.34 (brs, 1H), 8.97 (brs, 1H), 7.96 (s, 1H), 7.40 (s, 1H), 4.51-4.45 (t, J=6.2, 2H), 4.38 (d, J=13.4, 1H), 3.87 (d, J=13.1, 1H), 3.61 (t, J=6.6, 2H), 3.28-3.23 (m, 1H), 2.99-2.91 (m, 1H), 2.45 (t, J=6.6, 2H), 1.79-1.65 (m, 5H), 1.28-1.23 (m, 1H), 1.21 (d, J=7.0, 6H), 1.19-0.98 (m, 2H).

### Example 15

### 2-{[6-hydroxy-2-(1H-imidazol-1-yl)-5-(propan-2-yl)pyrimidin-4-yl]oxy}acetic acid

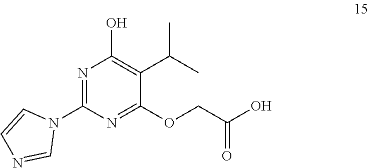 [SMILES: CC(C)c1c(O)nc(-n2ccnc2)nc1OCC(=O)O]

### Step 1: 2-((2-(1H-imidazol-1-yl)-5-isopropyl-6-((4-methoxybenzyl)oxy)pyrimidin-4-yl)oxy)acetic acid

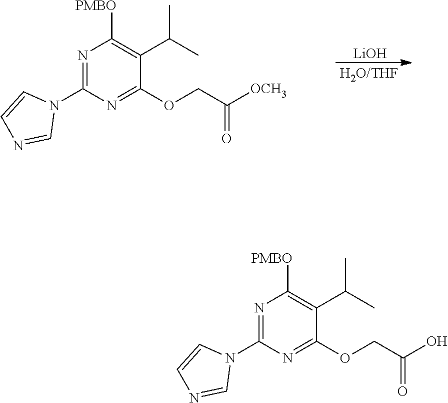

To a solution of methyl 2-((2-(1H-imidazol-1-yl)-5-isopropyl-6-((4-methoxybenzyl)-oxy)pyrimidin-4-yl)oxy)acetate (prepared by the method of Example 11 from methyl glycolate) (150 mg, 0.364 mmol) in THF (2 mL), and H₂O (1 mL) was added LiOH (8.71 mg, 0.364 mmol) and the resulting mixture was stirred at RT overnight. The volatiles were removed under reduced pressure and the residue was purified via silica gel chromatography (50-100% EtOAc in hexanes) to give the product (112 mg, 0.281 mmol, 77% yield) as a pale yellow solid.

MS (ES⁺) C₂₀H₂₂N₄O₅ requires: 398, found: 399 [M+H]⁺.

### Step 2: 2-{[6-hydroxy-2-(1H-imidazol-1-yl)-5-(propan-2-yl)pyrimidin-4-yl]oxy}acetic acid (15)

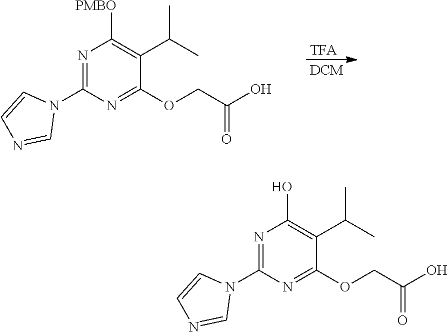

An aliquot of the material from the previous step was taken up in DCM (0.5 mL) and TFA (0.25 mL) was added. The mixture was stirred at RT for 1 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 12 min; Column: C18) to give the product 15 as a TFA salt.

MS (ES⁺) C₁₂H₁₄N₄O₄ requires: 278, found: 279 [M+H]⁺.

¹H NMR (600 MHz, DMSO-d₆) δ: 13.01 (brs, 1H), 12.55 (brs, 1H), 8.91 (s, 1H), 7.90 (s, 1H), 7.40 (s, 1H), 5.01 (s, 2H), 3.33-3.25 (m, 1H), 1.24 (d, J=7.0, 6H).

### Example 16

### 2-{[6-hydroxy-2-(1H-imidazol-1-yl)-5-(propan-2-yl)pyrimidin-4-yl]oxy}-1-(4-methylpiperazin-1-yl)ethan-1-one

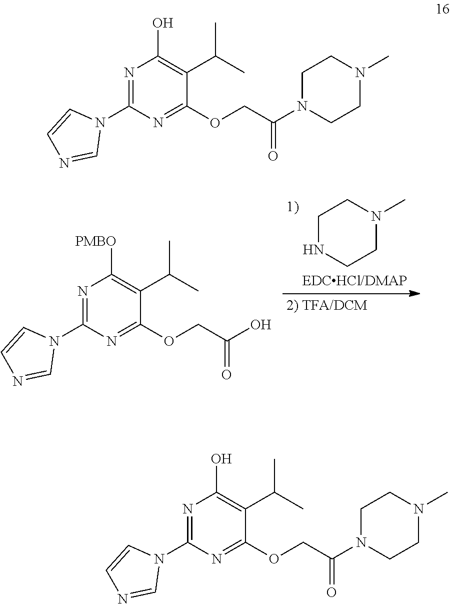

To a solution of 2-((2-(1H-imidazol-1-yl)-5-isopropyl-6-((4-methoxybenzyl)oxy)-pyrimidin-4-yl)oxy)acetic acid (Example 15, Step 1) (20 mg, 0.050 mmol) in DMF (0.5 mL) were added 1-methylpiperazine (10.06 mg, 0.100 mmol), EDC.HCl (19.25 mg, 0.100 mmol) and DMAP (12.27 mg, 0.100 mmol) and the resulting mixture was stirred at RT overnight. The solution was neutralized with 1M HCl then concentrated. DCM (0.5 mL) and TFA (0.4 mL) were added and the mixture was stirred for 1 h at RT. The volatiles were removed under reduced pressure. The residue was purified by mass-triggered preparative HPLC (Mobile phase: A=0.1% TFA/H₂O, B=0.1% TFA/MeCN; Gradient: B=10-40%; 12 min; Column: C18) to give the product 16 (3.6 mg, 6.2 μmol, 12% yield) as a white solid (bis-TFA salt).

MS (ES⁺) C₁₇H₂₄N₆O₃ requires: 360, found: 361 [M+H]⁺.

¹H NMR (600 MHz, DMSO-d₆+5 ul of TFA) δ: 10.04 (brs, 1H), 9.70 (s, 1H), 8.20 (s, 1H), 7.87 (s, 1H), 5.41 (d, J=12.8, 2H), 4.44-4.32 (m, 1H), 4.13-3.99 (m, 1H), 3.61-3.39 (m, 3H), 3.38-3.31 (m, 1H), 3.18-3.05 (m, 1H), 3.03-2.91 (m, 2H), 2.85 (s, 3H), 1.27 (d, J=7.0, 6H).

### Example 17

### 2-{[6-hydroxy-2-(1H-imidazol-1-yl)-5-(propan-2-yl)pyrimidin-4-yl]oxy}-N-methanesulfonylacetamide

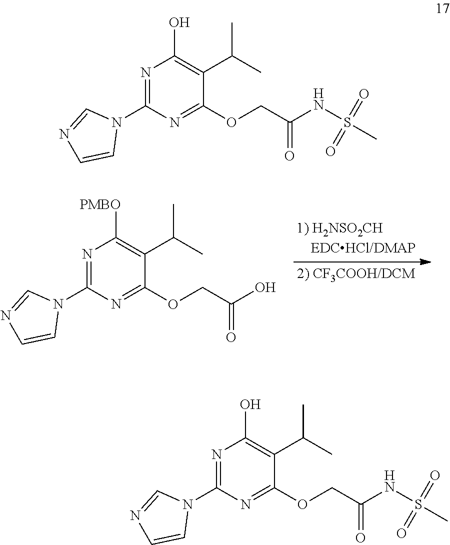

To a solution of 2-((2-(1H-imidazol-1-yl)-5-isopropyl-6-((4-methoxybenzyl)oxy)-pyrimidin-4-yl)oxy)acetic acid (Example 15, Step 1) (25 mg, 0.06 mmol) in DMF (0.5 mL) were added methanesulfonamide (12 mg, 0.12 mmol), EDC.HCl (24.0 mg, 0.12 mmol) and DMAP (15.3 mg, 0.12 mmol) and the resulting mixture was stirred at RT overnight. The mixture was taken up in EtOAc and treated with 1M HCl. The aqueous phase was extracted with EtOAc (3×) and the combined organic layers were concentrated under reduced pressure. To the residue was added DCM (0.5 mL) and TFA (0.4 mL) and stirred for 1 h at RT. The volatiles were removed under reduced pressure and the residue was purified by mass-triggered preparative HPLC (Mobile phase: A=0.1% TFA/H₂O, B=0.1% TFA/MeCN; Gradient: B=10-40%; 12 min; Column: C18) to give the product 17 (3.64 mg, 7.75 μmol, 12% yield) as a white solid (TFA salt).

MS (ES⁺) C₁₃H₁₇N₅O₅S requires: 355, found: 356 [M+H]⁺.

¹H NMR (600 MHz, DMSO-d₆) δ: 12.61 (brs, 1H), 12.16 (brs, 1H), 8.85 (s, 1H), 7.90 (s, 1H), 7.41 (s, 1H), 5.02 (s, 2H), 3.33-3.26 (m, 1H), 3.19 (s, 3H), 1.24 (d, J=7.0, 6H).

### Example 18

### 2-(1H-Imidazol-1-yl)-5-isopropyl-6-((pyridin-3-ylmethyl)amino)pyrimidin-4-ol

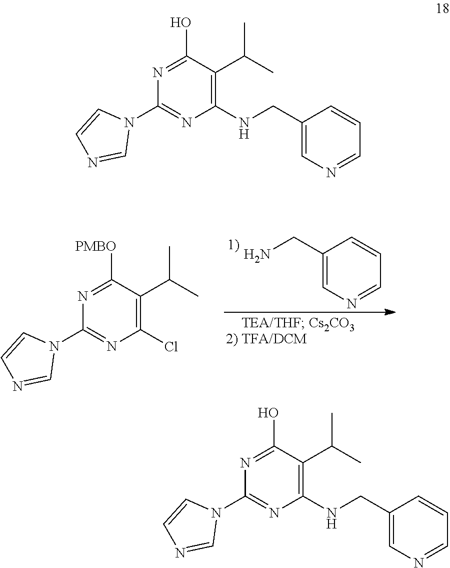

To a solution of 4-chloro-2-(1H-imidazol-1-yl)-5-isopropyl-6-((4-methoxybenzyl)-oxy)pyrimidine (Example 1, Step 5) (25 mg, 0.07 mmol) in THF (0.7 mL) were added TEA (9.7 μl, 0.07 mmol) followed by pyridin-3-ylmethanamine (7.5 mg, 0.070 mmol) and the resulting mixture was stirred at 120° C. for 2 days and at RT for 4 days. To the reaction was added Cs₂CO₃ (68.1 mg, 0.209 mmol) and heated to 120° C. for 1.5 h. The reaction was concentrated, diluted with DCM, filtered, and concentrated under a stream of N₂. The crude was dissolved in DCM (0.3 mL). TFA (0.3 mL) was added and the reaction was stirred at RT for 2.5 h. The reaction was concentrated under a stream of N₂, dissolved in DMSO (1 mL) and was purified by mass-triggered preparative HPLC (Mobile phase: A=0.1% TFA/H₂O, B=0.1% TFA/MeCN; Gradient: B=0-30%; 20 min; Column: C18) to give the product 18 as a white solid (bis-TFA salt).

MS (ES⁺) C₁₆H₁₈N₆O requires: 310, found: 311 [M+H]⁺.

¹H NMR 600 MHz, DMSO-d₆) δ: 11.67 (brs, 1H), 9.21 (brs, 1H), 8.77 (s, 1H), 8.59 (d, J=4.9, 1H), 8.12 (d, J=7.6, 1H), 7.97 (s, 1H), 7.84-7.79 (m, 1H), 7.61-7.67 (m, 1H), 7.56 (s, 1H), 4.75 (d, J=5.7, 2H), 3.14-3.07 (m, 1H), 1.25 (d, J=6.8, 6H).

### Example 19

### 5-(propan-2-yl)-6-(pyridin-3-ylmethoxy)-2-(1,3-thiazol-5-yl)pyrimidin-4-ol

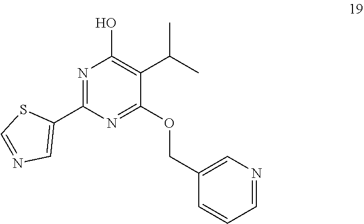 [SMILES: CC(C)c1c(O)nc(-c2cncs2)nc1OCc1cccnc1]

### Step 1: Thiazole-5-carboximidamide trifluoroacetate

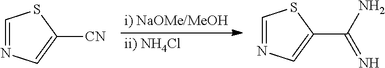 [SMILES: N#Cc1cncs1 ; N=C(N)c1cncs1]

To a solution of thiazole-5-carbonitrile (1000 mg, 9.08 mmol) in MeOH (10 mL) were added NaOMe (491 mg, 9.08 mmol). The mixture was stirred for 20 min (or until the methyl thiazole-5-carbimidate is observed) then NH₄Cl (486 mg, 9.08 mmol) was added and the resulting mixture was stirred at room temperature overnight. The reaction mixture was filtered, and the filtrate was concentrated under reduced pressure. The residue was purified by multiple mass-triggered preparative Large Column HPLC (Mobile phase: A=0.1% TFA/H₂O, B=0.1% TFA/MeCN; Gradient: B=5-20%; 16 min; Column: C18) to give the product (1062 mg, 4.40 mmol, 48% yield) as a pale yellow solid.

MS (ES⁺) C₄H₅N₃S requires: 127, found: 128 [M+H]⁺.

### Step 2: 5-Isopropyl-2-(thiazol-5-yl)pyrimidine-4,6(1H,5H)-dione

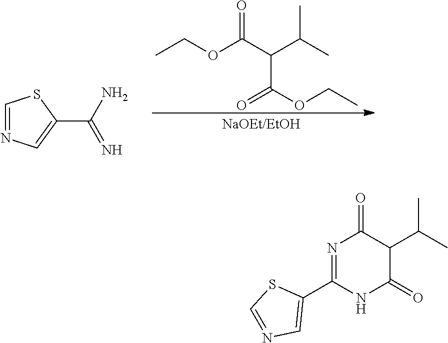 [SMILES: N=C(N)c1cncs1 ; CCOC(=O)C(C(=O)OCC)C(C)C ; CC(C)C1C(=O)N=C(c2cncs2)NC1=O]

To a solution of the material from the previous step (300 mg, 1.24 mmol) in ethanol (3 mL) were added NaOEt in EtOH (1.86 mL, 4.98 mmol; 21% w.t.) and diethyl 2-isopropylmalonate (302 mg, 1.49 mmol) and the resulting mixture was stirred at 55° C. overnight. The volatiles were removed under reduced pressure. The residue was treated with 1M HCl, whereupon a brown precipitate was formed. DCM was added and the layers were separated. The brown precipitate was removed by filtration, and after filtration, the solid was confirmed to be product. The aqueous phase was extracted with DCM (3×), and the combined organic layers and the solid were combined, concentrated under reduced pressure and purified via silica gel chromatography (0-20% MeOH in DCM) to give the product (194 mg, 0.818 mmol, 66% yield) as a yellow solid.

MS (ES⁺) C₁₀H₁₁N₃O₂S requires: 237, found: 238 [M+H]⁺.

### Step 3: 5-(4,6-Dichloro-5-isopropylpyrimidin-2-yl)thiazole

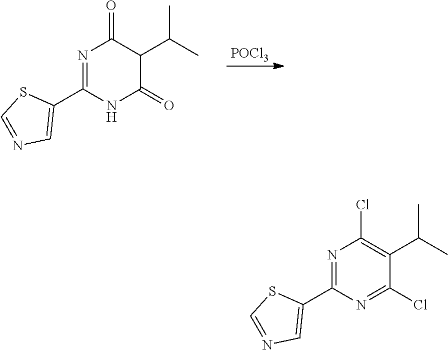

A suspension of the material from the previous step (194 mg, 0.818 mmol) in POCl₃ (1143 μl, 12.26 mmol) was stirred at 105° C. for 48 h. The solution was concentrated, the residue was taken up in EtOAc, saturated NaHCO₃ was added, and the layers were separated. The aqueous phase was extracted with EtOAc (3×) and the combined organic layers were concentrated under reduced pressure. The residue was purified via silica gel chromatography (0-40% EtOAc in hexanes) to give the product (148 mg, 0.540 mmol, 66% yield) as a yellow solid.

MS (ES⁺) C₁₀H₉Cl₂N₃S requires: 274, found: 275 [M+H]⁺.

### Step 4: 5-(4-Chloro-5-isopropyl-6-((4-methoxybenzyl)oxy)pyrimidin-2-yl)thiazole

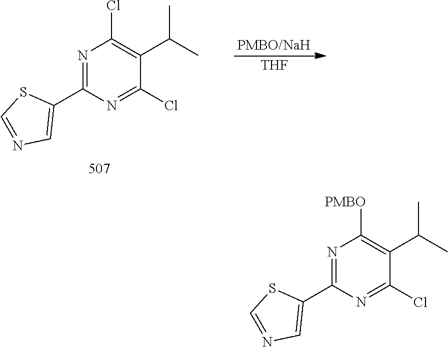

To a solution of the material from the previous step (142 mg, 0.518 mmol) in THF (2 mL) at −15° C. were added PMBOH (72 mg, 0.518 mmol) and NaH in 60% mineral oil (21 mg, 0.518 mmol), and the resulting mixture was stirred at room temperature for 2 h. The mixture was quenched with 1M HCl, EtOAc was added and the layers were separated. The aqueous phase was extracted with EtOAc (3×), the combined organic layers were concentrated under reduced pressure. The residue was purified via silica gel chromatography (0-40% EtOAc in hexanes) to give the product (183 mg, 0.487 mmol, 94% yield) as a yellow liquid.

MS (ES⁺) C₁₈H₁₈ClN₃O₂S requires: 375, found: 376 [M+H]⁺.

¹H NMR (600 MHz DMSO-d₆) δ: 9.27 (s, 1H), 8.66 (s, 1H), 7.48 (d, J=8.5, 2H), 6.97 (d, J=8.5, 2H), 5.50 (s, 2H), 3.75 (s, 3H), 3.44-3.36 (m, 1H), 1.24 (d, J=7.0, 6H)

### Step 5: 5-(Propan-2-yl)-6-(pyridin-3-ylmethoxy)-2-(1,3-thiazol-5-yl)pyrimidin-4-ol (19)

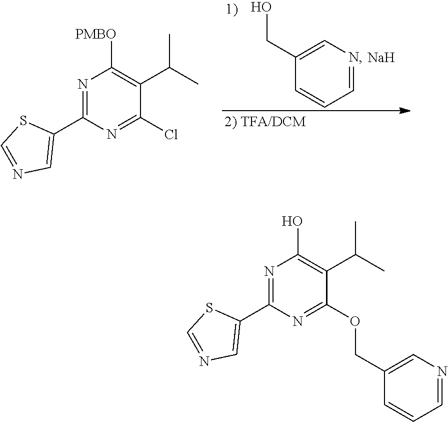

To a solution of the material from the previous step (25 mg, 0.067 mmol) in THF (0.5 mL) were added pyridin-3-ylmethanol (7.2 mg, 0.067 mmol) and NaH in 60% of mineral oil (2.6 mg, 0.067 mmol), and the resulting mixture was stirred under N₂ at RT for 3 h. Additional NaH in 60% of mineral oil (5.2 mg, 0.134 mmol) was added, the temperature was raised to 45° C., and the mixture was stirred for 2 h. The mixture was quenched with 1M HCl to pH-7, taken up in DCM and the layers were separated. The aqueous phase was extracted with DCM (3×), and the combined organic layers were concentrated under reduced pressure. The residue was dissolved in DCM (0.5 mL) and TFA (0.2 mL) was added and the mixture was stirred for 1 h. The volatiles were removed under reduced pressure. The residue was purified by mass-triggered preparative HPLC (Mobile phase: A=0.1% TFA/H₂O, B=0.1% TFA/MeCN; Gradient: B=20-50%; 12 min; Column: C18) to give the product 19 (10 mg, 0.023 mmol, 35% yield) as a white solid (TFA salt).

MS (ES⁺) C₁₆H₁₆N₄O₂S requires: 328, found: 329 [M+H]⁺.

¹H NMR (600 MHz, DMSO-d₆) δ: 9.25 (brs, 1H), 8.78 (s, 1H), 8.70 (brs, 1H), 8.62-8.59 (m, 1H), 8.08-8.02 (m, 1H), 7.60-7.54 (m, 1H), 5.51 (s, 2H), 3.28-3.21 (m, 1H), 1.20 (d, J=7.0, 6H).

### Example 20

### 6-(1-Benzyl-1H-pyrazol-4-yl)-5-(propan-2-yl)-2-(1,3-thiazol-5-yl)pyrimidin-4-ol

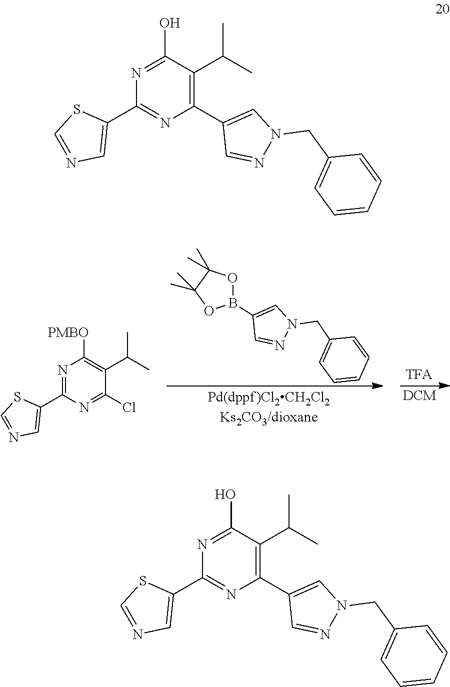

To a solution of 5-(4-chloro-5-isopropyl-6-((4-methoxybenzyl)oxy)pyrimidin-2-yl)-thiazole (25 mg, 0.067 mmol) (Example 19, Step 4) in dioxane (0.5 mL) were added 1-benzyl-4-(4,4,5,5-tetramethyl-1,3,2-dioxaborolan-2-yl)-1H-pyrazole (22.7 mg, 0.080 mmol), K₂CO₃ (18.3 mg, 0.133 mmol) and PdCl₂(dppf)-CH₂Cl₂ adduct (5.4 mg, 6.65 μmol). The reaction was degassed under N₂ and the resulting mixture was stirred at 80° C. overnight in a sealed vial. The mixture was directly purified via silica gel chromatography (0-40% EtOAc in hexanes) to give 5-(4-(1-benzyl-1H-pyrazol-4-yl)-5-isopropyl-6-((4-methoxybenzyl)oxy)pyrimidin-2-yl)thiazole as a colorless liquid. The residue was dissolved in DCM (0.5 mL) and TFA (0.2 mL) was added the mixture was stirred for 1 h then concentrated in-vacuo. The residue was purified by mass-triggered preparative HPLC (Mobile phase: A=0.1% TFA/H₂O, B=0.1% TFA/MeCN; Gradient: B=30-70%; 12 min; Column: C18) to give the product 20 (0.34 mg, 0.90 μmol, 1.3% yield) as a white solid.

MS (ES⁺) C₂₀H₁₉N₅OS requires: 377, found: 378 [M+H]⁺.

¹H NMR (600 MHz, DMSO-d₆) δ: 12.8 (brs, 1H), 9.26 (s, 1H), 8.80 (s, 1H), 8.25 (s, 1H), 7.79 (s, 1H), 7.39-7.34 (m, 2H), 7.32-7.28 (m, 2H), 6.51 (s, 1H), 5.42 (s, 2H), 3.26-3.16 (m, 1H), 1.33 (d, J=6.7, 6H).

### Example 21

### 2-(1H-imidazol-4-yl)-5-isopropyl-6-methylpyrimidin-4-ol

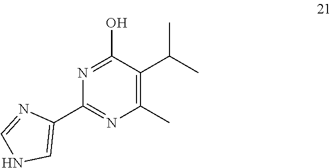 [SMILES: Cc1nc(-c2c[nH]cn2)nc(O)c1C(C)C]

### Step 1: 5-Isopropyl-6-methylpyrimidine-2,4(1H,3H)-dione

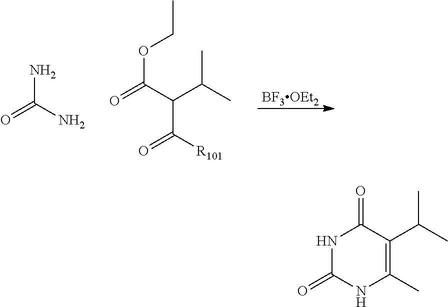

To a microwave vial containing ethyl 2-acetyl-3-methylbutanoate (0.5 g, 3.2 mmol) with BF₃-Et₂O (0.49 mL, 3.8 mmol) was added urea (0.25 g, 4.16 mmol) and the mixture was stirred at RT for 1 min. The vial was purged with N₂, sealed, and the reaction was irradiated in a microwave reactor (135° C.) for 50 min. The reaction was dissolved in a mixture of DCM and MeOH, adsorbed onto silica gel and purified via flash chromatography (20-100% EtOAc in hexanes) to give the product (114 mg, 0.68 mmol, 21% yield) as an off-white solid.

MS (ES⁺) C₈H₁₂N₂O₂ requires: 168, found: 169 [M+H]⁺.

### Step 2: 2,4-Dichloro-5-isopropyl-6-methylpyrimidine

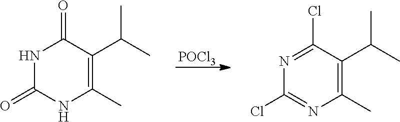

To the material from the previous step (270 mg, 1.605 mmol) was added POCl₃ (150 al, 1.605 mmol), and the resulting mixture was stirred at 105° C. (reflux) for 14 h. The reaction was cooled to RT, and poured in a mix of ice and saturated NaHCO₃ (200 mL). The mixture when basic was extracted with DCM (3×100 mL). The organic layers were combined, washed with brine, dried over MgSO₄, filtered, and concentrated to give the product (330 mg, 1.609 mmol, 100% yield) as a yellow liquid.

MS (ES⁺) C₈H₁₀Cl₂N₂ requires: 204, found: 205 [M+H]⁺.

### Step 3: 2-Chloro-5-isopropyl-4-((4-methoxybenzyl)oxy)-6-methylpyrimidine

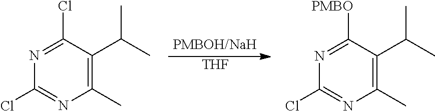

To a solution of (4-methoxyphenyl)methanol (50.5 mg, 0.366 mmol) and the material from the previous step (150 mg, 0.731 mmol) in THF (1 mL) under nitrogen at −20° C. was added NaH in 60% mineral oil (17.5 mg, 0.731 mmol), and the resulting mixture was stirred at −20° C. to −10° C. for 10 min then allowed to warm to RT for 2.5 h. Reaction was quenched with saturated NH₄Cl (2 mL). The reaction was diluted with water (10 mL) and extracted with EtOAc (2×12 mL). The organic layers were washed with saturated NaCl, combined, dried over MgSO₄, concentrated and purified via silica gel chromatography (0-20% EtOAc in hexanes) to give the product (55 mg, 0.179 mmol, 49% yield) as a colorless liquid.

MS (ES⁺) C₁₆H₁₉ClN₂O₂ requires: 306, found: 307 [M+H]⁺.

¹H NMR (600 MHz; DMSO-d₆) δ: 7.41 (d, J=8.3, 2H), 6.96 (d, J=8.3, 2H), 5.33 (s, 2H), 3.76 (s, 3H), 3.15 (m, 1H), 2.42 (s, 3H), 1.18 (d, J=6.8, 6H).

### Step 4: 2-(1H-imidazol-4-yl)-5-isopropyl-6-methylpyrimidin-4-ol (21)

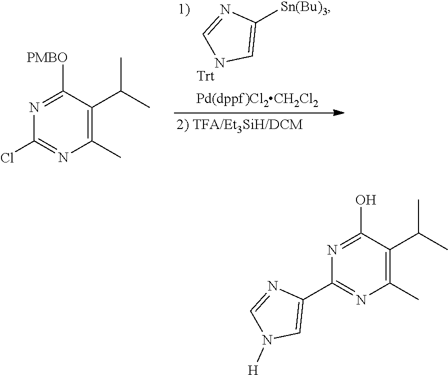

A mixture of the material from the previous step (25 mg, 0.081 mmol), 4-(tributyl-stannyl)-1-trityl-1H-imidazole (59 mg, 0.098 mmol), and PdCl₂(dppf)-CH₂Cl₂ adduct (13 mg, 0.016 mmol) was heated at 120° C. overnight, then directly purified via silica gel chromatography (0-40% EtOAc in hexanes to give 5-isopropyl-4-((4-methoxybenzyl)oxy)-6-methyl-2-(1-trityl-1H-imidazol-4-yl)pyrimidine as a yellow liquid. The residue was dissolved in DCM (0.8 mL) and TFA (0.2 mL) and Et₃SiH (13 μl, 0.081 mmol) were added. The mixture was stirred 30 min, then evaporated and purified by mass-triggered preparative HPLC (Mobile phase: A=0.1% TFA/H₂O, B=0.1% TFA/MeCN; Gradient: B=10-30%; 20 min; Column: C18) to give the product 21 (2.8 mg, 10% yield) as a white solid (TFA salt).

MS (ES⁺) C₁₁H₁₄N₄O requires: 218, found: 219 [M+H]⁺.

¹H NMR (600 MHz, DMSO-d₆) δ: 8.55 (brs, 1H), 8.20 (s, 1H), 3.10-3.01 (m, 1H), 2.37 (s, 3H), 1.25 (d, J=6.9, 6H).

### Example 22

### 2-(1H-imidazol-1-yl)-5-isopropyl-6-methylpyrimidin-4-ol

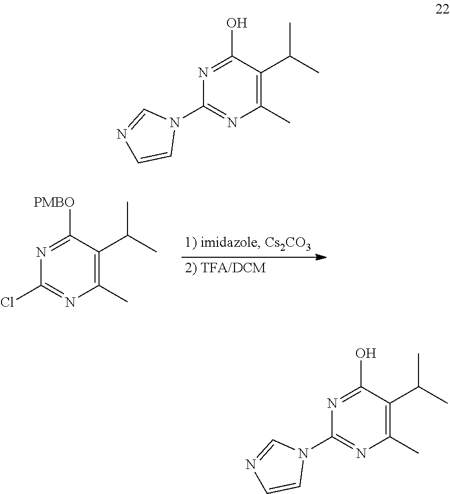

A mixture of 2-chloro-5-isopropyl-4-((4-methoxybenzyl)oxy)-6-methylpyrimidine (25 mg, 0.081 mmol) (Example 21, Step 3), 1H-imidazole (28 mg, 0.40 mmol), and Cs₂CO₃ (53 mg, 0.163 mmol) was heated at 120° C. for 4 h. Additional 1H-imidazole (14 mg, 0.20 mmol) was added and the mixture was left at the same temperature overnight. Additional 1H-imidazole (22 mg, 0.32 mmol) was added and the mixture was left 6 h, then diluted with water and extracted with EtOAc. The organic phase was concentrated under reduced pressure, and the residue was dissolved in DCM (0.7 mL) and TFA (0.3 mL). The mixture was stirred 1 h then evaporated and purified by mass-triggered preparative HPLC (Mobile phase: A=0.1% TFA/H₂O, B=0.1% TFA/MeCN; Gradient: B=10-40%; 20 min; Column: C18) to give the product 22 (1.4 mg, 4.21 μmol, 5% yield) as a white solid (TFA salt).

MS (ES⁺) C₁₁H₁₄N₄O requires: 218, found: 219 [M+H]+.

¹H NMR (600 MHz, DMSO-d₆) δ: 12.79 (brs, 1H), 9.10 (brs, 1H), 7.99 (s, 1H), 7.49 (s, 1H), 3.23-3.16 (m, 1H), 2.48 (s, 3H), 1.26 (d, J=6.9, 6H).

### Example 23

### 2-(2-aminoimidazol-1-yl)-5-isopropyl-6-methyl-pyrimidin-4-ol

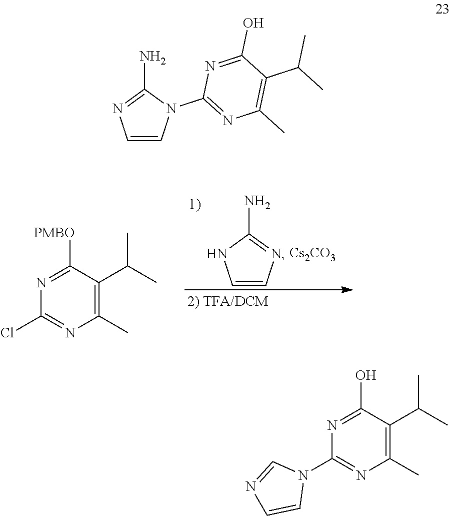

A mixture of 1H-imidazol-2-amine (200 mg, 2.41 mmol) and 2-chloro-5-isopropyl-4-[(4-methoxyphenyl)methoxy]-6-methyl-pyrimidine (Example 21, Step 3) (147 mg, 482 μmol) was dissolved in DMF (3 mL), then Cs₂CO₃ (314 mg, 964 μmol) was added, and the mixture was stirred at 120° C. for 16 h. H₂O (10 mL) was added and the mixture was freeze dried to afford a brown residue, to which was added DCM (10 mL). The material was washed with water (5 mL). The organic layer was dried over Na₂SO₄, filtered, concentrated to afford a brown oil which was used directly in the next step.

To a solution of the residue (100 mg, 0.28 mmol) in DCM (3 mL) was added TFA (1 mL), then the mixture was stirred at 15° C. for 3 h. The reaction mixture was concentrated and the residue was purified by prep-TLC (Petroleum Ether:EtOAc=2:1) to obtain a colorless oil (50 mg). A second purification was performed by prep-HPLC (column: Phenomenex Synergi C18 150*25*10 um; mobile phase: A=0.1% TFA/H₂O, B=0.1% TFA/MeCN Gradient: B=15-45%; 11 min) to afford the product 23 as a TFA salt (3.4 mg, 9.8 μmol, 3.5% yield).

MS (ES⁺) C₁₁H₁₅N₅O requires: 233, found: 234 [M+H]⁺.

¹H NMR (400 MHz, Methanol-d₄): δ 7.68 (d, J=2.4, 1H), 6.94 (d, J=2.8, 1H), 3.33-3.27 (m, 1H), 2.5 (s, 3H), 1.35 (d, J=7.2, 6H).

### Example 24a/b

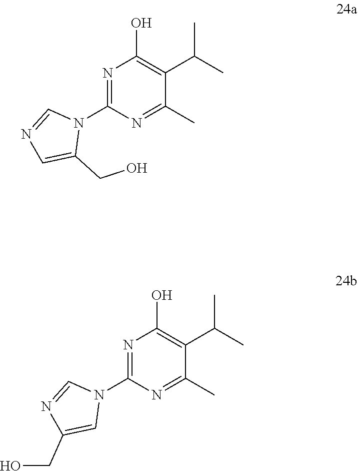 [SMILES: Cc1nc(-n2cncc2CO)nc(O)c1C(C)C ; Cc1nc(-n2cnc(CO)c2)nc(O)c1C(C)C]

### Step 1: [3-[5-isopropyl-4-[(4-methoxyphenyl)methoxy]-6-methylpyrimidin-2-yl]imidazol-4-yl]-methanol

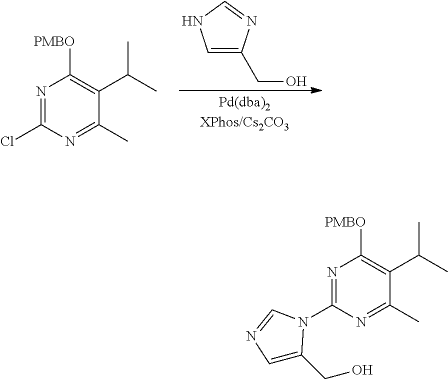

To a solution of 1H-imidazol-5-yl-methanol (320 mg, 3.26 mmol) and 2-chloro-5-isopropyl-4-[(4-methoxyphenyl)methoxy]-6-methyl-pyrimidine (100 mg, 326 μmol) (Example 21, Step 3) in toluene (1.5 mL) was added Pd(dba)₂ (18.7 mg, 32.6 μmol), XPhos (15.56 mg, 32.6 μmol) and Cs₂CO₃ (106 mg, 326 μmol) at 15° C. The suspension was degassed under vacuum and purged with N₂ several times. The reaction mixture was stirred at 100° C. for 16 h, then concentrated in vacuum. The crude product was purified by prep-TLC (Petroleum Ether:EtOAc=2:1) to give the product (13 mg, 33 μmol, 10% yield) as a colorless liquid. The other isomer was not collected.

MS (ES⁺) C₂₀H₂₄N₄O₃ requires: 368, found: 369 [M+H]⁺.

### Step 2: 2-[5-(hydroxymethyl)imidazol-1-yl]-5-isopropyl-6-methyl-pyrimidin-4-ol (24a)

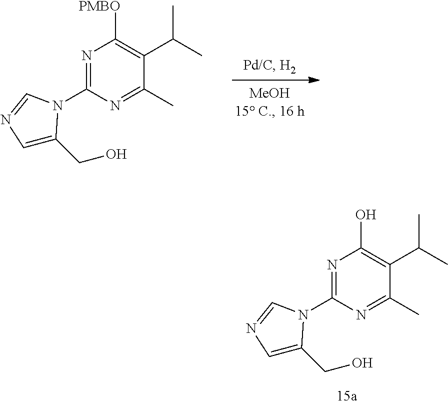

To the material from the previous step (13 mg, 35.2 μmol) in MeOH (1 mL) was added 10% Pd/C (50 mg, 35.28 μmol) at 15° C. The suspension was degassed under vacuum and purged with H₂ several times. The mixture was stirred under H₂ (16 psi) at 15° C. for 1 h. The reaction mixture was filtered and the filtrate was concentrated in vacuum to give the product 24a (3.7 mg, 13 μmol, 39% yield) as a white solid.

MS (ES⁺) C₁₂H₁₆N₄O₂ requires: 248, found: 249 [M+H]⁺.

¹H NMR (400 MHz, CDCl₃) δ: 9.20 (s, 1H), 7.13 (s, 1H), 4.72 (s, 2H), 3.37-3.15 (m, 1H), 2.56 (s, 3H), 1.42 (d, J=7.0, 6H).

### Step 3: [1-[5-isopropyl-4-[(4-methoxyphenyl)methoxy]-6-methyl-pyrimidin-2-yl]imidazol-4-yl]-methanol

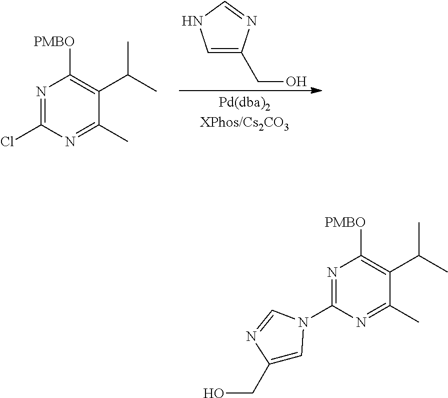

To a solution of 1H-imidazol-5-ylmethanol (319 mg, 3.26 mmol) and 2-chloro-5-isopropyl-4-[(4-methoxyphenyl)methoxy]-6-methyl-pyrimidine (100 mg, 325 μmol) (Example 21, Step 3) in toluene (1.5 mL) was added Pd(dba)₂ (18.7 mg, 32.6 μmol), XPhos (15.5 mg, 32.60 μmol) and Cs₂CO₃ (106 mg, 325.96 μmol) at 15° C. The suspension was degassed under vacuum and purged with N₂ several times. The reaction mixture was stirred at 100° C. for 16 h. The mixture was concentrated in vacuum. The crude product was purified by prep-TLC (DCM:MeOH=2:1) to give the product (13 mg, 31 μmol, 9% yield) as a white solid. The other isomer was not collected.

¹H NMR (400 MHz, CDCl₃) δ: 8.63 (s, 1H), 7.81 (s, 1H), 7.39 (d, J=8.4, 2H), 6.92 (d, J=8.4, 2H), 5.42 (s, 2H), 4.68 (s, 2H), 3.82 (s, 3H), 3.15-3.30 (m, 1H), 2.49 (s, 3H), 1.29 (d, J=6.8, 6H).

### Step 4: 2-[4-(hydroxymethyl)imidazol-1-yl]-5-isopropyl-6-methyl-pyrimidin-4-ol (24b)

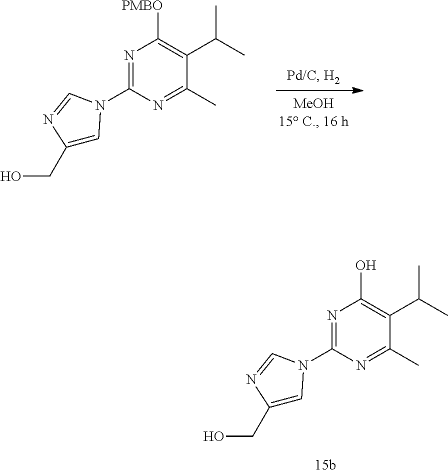

To a solution of the material from the previous step (13 mg, 35.2 μmol) in MeOH (1 mL) was added 10% Pd/C (10 mg) at 15° C. The suspension was degassed under vacuum and purged with H₂ several times. The mixture was stirred under H₂ (16 psi) at 15° C. for 16 h. The reaction mixture was filtered and the filtrate was concentrated in vacuum. The crude product was purified by prep-TLC (DCM:MeOH=8:1) to give the product 24b (3.1 mg, 11.61 μmol, 33% yield) as a white solid.

MS (ES⁺) C₁₂H₁₆N₄O₂ requires: 248, found: 249 [M+H]⁺.

¹H NMR (400 MHz, CDCl₃) δ: 8.54 (s, 1H), 7.86 (s, 1H), 4.59 (s, 2H), 3.30-3.24 (m, 1H), 2.51 (s, 3H), 1.35 (d, J=7.0, 6H).

### Example 25

### 2-[2-(hydroxymethyl)imidazol-1-yl]-5-isopropyl-6-methyl-pyrimidin-4-ol

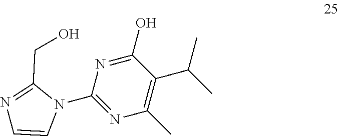 [SMILES: Cc1nc(-n2ccnc2CO)nc(O)c1C(C)C]

### Step 1: 1H-Imidazol-2-ylmethoxy(triisopropyl)silane

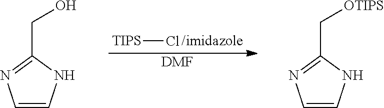

To a solution of 1H-imidazol-2-ylmethanol (500 mg, 5.1 mmol) in DMF (15 mL) was added imidazole (346 mg, 5.1 mmol) and TIPSCl (1.08 g, 5.61 mmol, 1.2 mL) at 15° C. The reaction mixture was stirred at 15° C. for 2 h. H₂O (20 mL) was added, and the aqueous phase was extracted with EtOAc (20 mL×2). The combined organic phase was washed with brine (30 mL×2), dried over Na₂SO₄ and concentrated in vacuum. The residue was purified by column chromatography on silica gel (DCM:MeOH=10:1) to give the product (600 mg, 2.3 mmol, 46% yield) as a colorless liquid.

¹H NMR (400 MHz, CDCl₃): δ 6.95 (s, 2H), 4.85 (s, 2H), 1.10 (m, 3H), 1.00 (d, J=6.8, 18H).

### Step 2: Triisopropyl-[[1-[5-isopropyl-4-[(4-methoxyphenyl)methoxy]-6-methyl-pyrimidin-2-yl]imidazol-2-yl]methoxy]silane

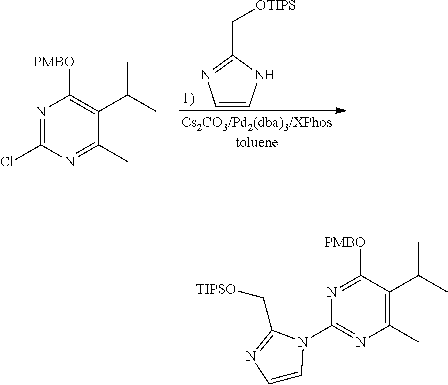

To a solution of the material from the previous step (300 mg, 1.18 mmol) and 2-chloro-5-isopropyl-4-[(4-methoxyphenyl)methoxy]-6-methylpyrimidine (362 mg, 1.18 mmol) in toluene (20 mL) was added Cs₂CO₃ (768 mg, 2.3 mmol), Pd₂(dba)₃ (108 mg, 118 μmol) and XPhos (56 mg, 118 μmol) at 15° C. The suspension was degassed under vacuum and purged with N₂ several times. The reaction mixture was stirred at 100° C. for 16 h. The reaction mixture was concentrated in vacuum. The crude product was purified by prep-TLC (Petroleum ether:EtOAc=10:1) to give the product (130 mg, 235 μmol, 20% yield) as a light yellow liquid which was used in the next step directly.

MS (ES⁺) C₂₉H₄₄N₄O₃Si requires: 524, found: 525 [M+H]⁺.

### Step 3: 5-isopropyl-6-methyl-2-[2-(triisopropylsilyloxymethyl)imidazol-1-yl] pyrimidin-4-ol

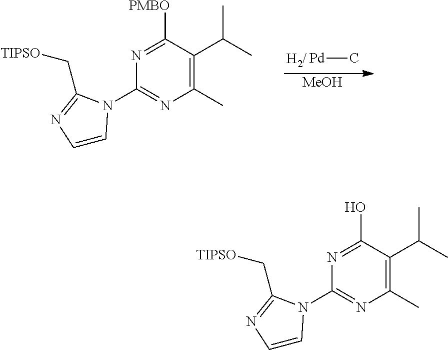

To a solution of the material from the previous step (120 mg, 228 μmol) in MeOH (10 mL) was added 10% Pd/C (30 mg, 22.8 μmol) at 15° C. The suspension was degassed under vacuum and purged with H₂ several times. The mixture was stirred under H₂ (16 psi) at 15° C. for 16 h. The reaction mixture was filtered, and the filtrate was concentrated to give the product (76 mg, 172.8 μmol, 75% yield) as a white solid which was used in next step directly.

¹H NMR (400 MHz, Methanol-d₄) δ 7.66 (d, J=1.2, 1H), 6.99 (d, J=1.2, 1H), 5.24 (s, 2H), 3.24-3.27 (m, 1H), 2.47 (s, 3H), 1.35 (d, J=6.8, 6H), 1.06-1.01 (m, 3H), 0.98 (d, J=6.4, 18H).

### Step 4: 2-[2-(Hydroxymethyl)imidazol-1-yl]-5-isopropyl-6-methyl-pyrimidin-4-ol (25)

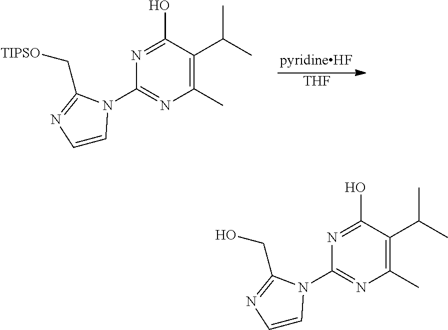

To a solution of the material from the previous step (40 mg, 98.8 μmol) in THF (10 mL) was added pyridine.HF (220 mg, 2.22 mmol, 0.2 mL) at 15° C. The reaction mixture was stirred at 15° C. for 2 h. The reaction mixture was concentrated in vacuum. The crude product was purified by prep-TLC (DCM:MeOH=8:1) to give the product 25 (5.7 mg, 21 μmol, 22% yield) as a white solid.

MS (ES⁺) C₁₂H₁₆N₄O₂ requires: 248, found: 249 [M+H]⁺.

¹H NMR (400 MHz, Methanol-d₄) δ 7.96 (s, 1H), 7.06 (s, 1H), 4.94 (s, 2H), 3.05-3.08 (m, 1H), 2.53 (s, 3H), 1.36 (d, J=7.0, 6H).

### Example 26

### 5-Isopropyl-6-methyl-2-(1H-pyrazol-4-yl)pyrimidin-4-ol

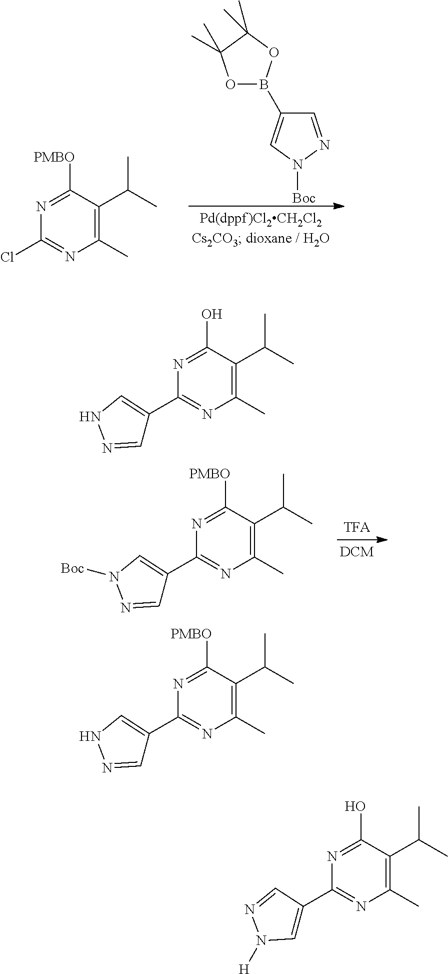

To a vial containing tert-butyl 4-(4,4,5,5-tetramethyl-1,3,2-dioxaborolan-2-yl)-1H-pyrazole-1-carboxylate (36.0 mg, 0.122 mmol), 2-chloro-5-isopropyl-4-((4-methoxybenzyl)oxy)-6-methylpyrimidine (Example 21, Step 3) (25 mg, 0.081 mmol), Cs₂CO₃ (80 mg, 0.244 mmol), and PdCl₂(dppf)-CH₂Cl₂ adduct (9.98 mg, 0.012 mmol), was added dioxane (0.4 mL, previously degassed with N₂) and H₂O (5 μl, 0.24 mmol). The mixture was degassed with N₂ and heated at 90° C. for 15 h. The reaction was diluted with DCM, filtered, supported on silica gel, and purified via silica gel chromatography (0-100% EtOAc in hexanes) to give two products: tert-butyl 4-(5-isopropyl-4-((4-methoxybenzyl)oxy)-6-methylpyrimidin-2-yl)-1H-pyrazole-1-carboxylate (6 mg, 0.014 mmol, 16% yield) as a white solid, and 5-isopropyl-4-((4-methoxybenzyl)oxy)-6-methyl-2-(1H-pyrazol-4-yl)pyrimidine (10.8 mg, 0.032 mmol, 39% yield) as an off white solid. The tert-butyl 4-(5-isopropyl-4-((4-methoxybenzyl)oxy)-6-methylpyrimidin-2-yl)-1H-pyrazole-1-carboxylate was dissolved in DCM (0.6 mL), TFA (0.3 mL) was added, and the reaction was stirred at RT for 24 h. The reaction was concentrated, azeotroped with DCM/toluene mixtures and purified by mass-triggered preparative HPLC (Mobile phase: A=0.1% TFA/H₂O, B=0.1% TFA/MeCN; Gradient: B=20-60%; 20 min; Column: C18) to give the product 26 (3.0 mg, 0.014 mmol, 17% yield) as a white solid.

MS (ES⁺) C₁₁H₁₄N₄O requires: 218, found: 219 [M+H]⁺.

¹H NMR (600 MHz, Methanol-d₄) δ: 8.27 (s, 2H), 3.04-2.97 (m, 1H), 2.32 (s, 3H), 1.19 (d, J=7.2, 6H).

### Example 27

### 2-[1-(2-hydroxyethyl)-1H-imidazol-5-yl]-6-methyl-5-(propan-2-yl)pyrimidin-4-ol

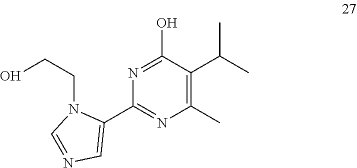 [SMILES: Cc1nc(-c2cncn2CCO)nc(O)c1C(C)C]

### Step 1: 2-Bromoethoxy-tert-butyl-diphenyl-silane

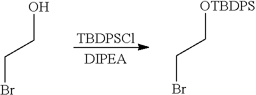

To a mixture of 2-bromoethanol (11 g, 88.0 mmol, 6.25 mL), DIPEA (17 g, 131.5 mmol, 22.9 mL) in DCM (40 mL) was added tert-butyl(chloro)diphenylsilane (26 g, 94.5 mmol, 24.3 mL) at 0° C. The mixture was then stirred at 15° C. for 2 h. DCM (60 mL) was added. The organic phase was washed with saturated NaHCO₃ solution (50 mL), followed by 1 N HCl solution (50 mL), and then brine (50 mL). The organic layer was dried over Na₂SO₄ and concentrated, and the residue was purified by column chromatograph (Petroleum Ether/EtOAc=30/1) to afford the product as a colorless oil (30 g).

¹H NMR (400 MHz, CDCl₃) δ: 7.61-7.58 (m, 4H), 7.37-7.31 (m, 6H), 3.85 (t, J=8.8, 2H), 3.35 (t, J=8.8, 2H), 0.99 (s, 9H).

### Step 2: tert-Butyl-(2-imidazol-1-ylethoxy)-diphenyl-silane

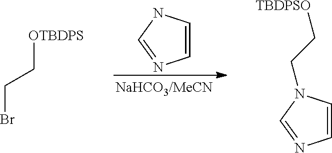

To a mixture of imidazole (22 g, 322.8 mmol) and the material from the previous step (30 g, 82.5 mmol) in MeCN (250 mL) was added NaHCO₃ (21 g, 250 mmol, 9.72 mL). The mixture was stirred at 60° C. for 16 h. The reaction mixture was filtered, and the filtrate was concentrated. To the residue was added H₂O (150 mL), then it was extracted with EtOAc (100 mL×2). The organic layer was washed with brine (100 mL), dried over Na₂SO₄ and concentrated, and the residue was purified by column chromatograph (Petroleum Ether/EtOAc=5/1) to afford the product (16 g) as a colorless oil.

MS (ES⁺) C₂₁H₂₆N₂OSi requires: 350, found: 351 [M+H]⁺.

### Step 3: 2-(5-Bromoimidazol-1-yl)ethoxy-tert-butyl-diphenyl-silane

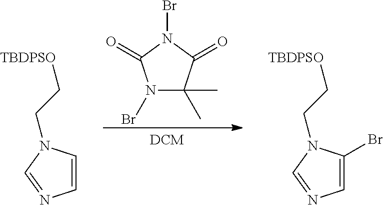

To a mixture of the material from the previous step (5 g, 14.26 mmol) in DCM (100 mL) was added 1,3-dibromo-5,5-dimethyl-imidazolidine-2,4-dione (2.24 g, 7.84 mmol) at −30° C. The mixture was slowly warmed to 15° C. and stirred for another 2 h. The reaction mixture was quenched with saturated Na₂SO₃ solution (100 mL), then concentrated to remove DCM, the residue was refluxed for another 16 h. Then it was extracted with EtOAc (50 mL×2), the organic layer was washed with brine (50 mL), dried over Na₂SO₄, filtered and concentrated. The residue was purified by column chromatograph (Petroleum Ether/EtOAc=1/1) to obtain the product (2.1 g) as a yellow solid.

MS (ES⁺) C₂₁H₂₅BrN₂OSi requires: 428/430, found: 429/431 [M+H]⁺.

¹H NMR (400 MHz, CDCl₃) δ: 7.66 (s, 1H), 7.54-7.39 (m, 10H), 7.05 (s, 1H), 4.08 (t, J=5.2, 2H), 3.85 (t, J=5.2, 2H), 1.0 (s, 9H).

### Step 4: [3-[2-[tert-Butyl (diphenyl)silyl]oxyethyl]imidazol-4-yl]boronic acid

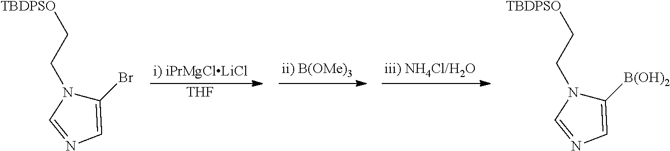

To a mixture of material from the previous step (500 mg, 1.16 mmol) in THF (5 mL) was added iPrMgCl′LiCl complex solution (1.3 M, 1.80 mL) at 0° C. under N₂ atmosphere. The mixture was then stirred at this temperature for 1 h. B(OMe)₃ (362 mg, 3.48 mmol, 393 μl) was added and the mixture was stirred at 15° C. for 16 h. Saturated NH₄Cl solution (10 mL) was added to the mixture, the aqueous layer was extracted with EtOAc (10 mL×2), and the organic layer was dried over Na₂SO₄ and concentrated to afford the product (560 mg, crude) as a colorless oil which was used directly in the next step.

MS (ES⁺) C₂₁H₂₇BN₂O₃Si requires: 394, found: 395 [M+H]⁺.

### Step 5: tert-Butyl-[2-[5-[5-isopropyl-4-[(4-methoxyphenyl)methoxy]-6-methylpyrimidin-2-yl]-imidazol-1-yl]ethoxy]-diphenyl-silane

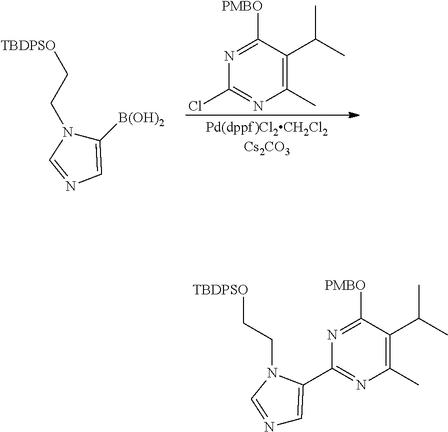

To a mixture of the material from the previous step (560 mg, 1.42 mmol), 2-chloro-5-isopropyl-4-[(4-methoxyphenyl) methoxy]-6-methyl-pyrimidine (435 mg, 1.42 mmol), Cs₂CO₃ (925 mg, 2.84 mmol) in dioxane (7 mL), H₂O (2 mL) was added Pd(dppf)Cl₂.CH₂Cl₂ adduct (232 mg, 284 μmol). The mixture was purged with N₂ and then stirred at 60° C. for 16 h. To the reaction was added saturated NH₄Cl solution (10 mL), and the aqueous layer was extracted with EtOAc (20 mL×2). The combined organic layers were dried over Na₂SO₄ and concentrated. The residue was purified by column chromatograph (Petroleum ether/EtOAc=5/1-2/1) to obtain the product (300 mg, 34% yield) as a yellow oil.

MS (ES⁺) C₃₇H₄₄N₄O₃Si requires: 620, found: 621 [M+H]⁺.

¹H NMR (400 MHz, CDCl₃) δ: 7.92 (s, 1H), 7.63 (s, 1H), 7.51-7.49 (m, 4H), 7.37-7.31 (m, 8H), 6.90 (d, J=8.8, 2H), 5.35 (s, 2H), 4.69 (t, J=5.2, 2H), 3.94 (t, J=5.2, 2H), 3.18-3.14 (m, 1H), 1.27 (d, J=1.6, 6H) 1.00 (s, 9H).

### Step 6: 2-[5-[5-Isopropyl-4-[(4-methoxyphenyl)methoxy]-6-methylpyrimidin-2-yl]imidazol-1-yl]ethanol

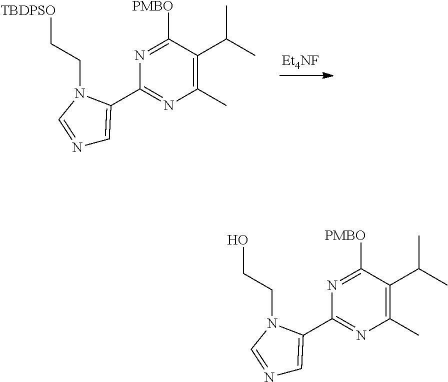

To a mixture of the material from the previous step (150 mg, 241 μmol) in dioxane (2 mL) was added Et₄NF (72 mg, 483 μmol). The mixture was then stirred at 60° C. for 6 h. H₂O (5 mL) was added to the reaction, the mixture was extracted with EtOAc (10 mL×2), and the organic layer was dried over Na₂SO₄ and concentrated to afford the product (100 mg). The residue was used directly in the next step without purification.

MS (ES⁺) C₂₁H₂₆N₄O₃ requires: 382, found: 383 [M+H]⁺.

### Step 7: 2-[3-(2-Hydroxyethyl)imidazol-4-yl]-5-isopropyl-6-methylpyrimidin-4-ol (27)

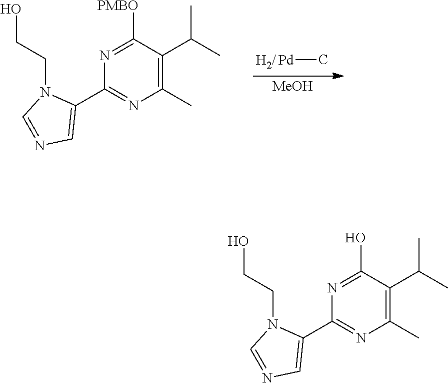

To a mixture of the material from the previous step (100 mg, 261 μmol) in MeOH (2 mL) was added Pd/C (135 mg, 261 μmol, 10%) under an N₂ atmosphere. The mixture was purged with H₂ and then stirred at 50° C. under H₂ (15 psi) atmosphere for 2 h. The reaction was filtered, and the filtrate was concentrated. The residue was purified by prep-TLC (DCM/MeOH=10/1) to afford the product 27 (35 mg, 51% yield) as a off-white solid.

MS (ES⁺) C₁₃H₁₈N₄O₂ requires: 262, found: 263 [M+H]⁺.

¹H NMR (400 MHz, CDCl₃) δ: 7.92 (s, 1H), 7.77 (s, 1H), 4.63 (t, J=5.2, 2H), 3.89 (t, J=5.2, 2H), 3.14-3.19 (m, 1H), 2.39 (s, 3H), 1.35 (d, J=6.8, 6H).

### Example 28a/b

### 2-(1H-imidazol-1-yl)-5-isopropyl-6-(pyridin-3-yloxy)pyrimidin-4-ol 2,6-di(1H-imidazol-1-yl)-5-isopropylpyrimidin-4-ol

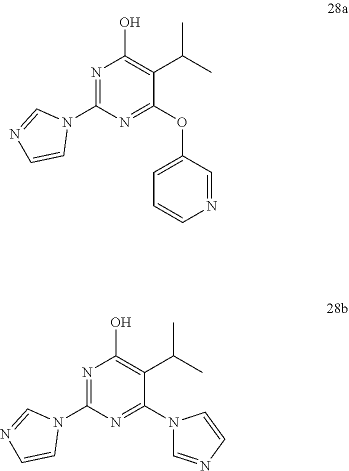 [SMILES: CC(C)c1c(O)nc(-n2ccnc2)nc1Oc1cccnc1 ; CC(C)c1c(O)nc(-n2ccnc2)nc1-n1ccnc1]

### Step 1: 2,4,6-Trichloro-5-isopropylpyrimidine

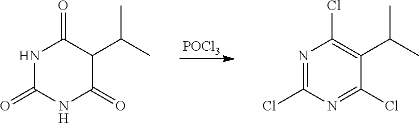

To a round bottom flask containing 5-isopropylbarbituric acid (5.0 g, 29 mmol) was added POCl₃ (11 mL, 118 mmol), and the mixture was cooled in an ice bath. To this solution was added N,N-diethylaniline (14 mL, 88 mmol) dropwise and the mixture was stirred and allowed to warm to RT for 1 h. The reaction was heated at 145° C. for 15 h. The reaction was cooled to room temperature, poured into a mixture of water and crushed ice, and the pale yellow precipitate mixture stirred until the ice melted. The precipitate was filtered, washed with H₂O (×3). The solid was then dissolved in hexanes, washed with water (×2), and with saturated NaCl, dried over MgSO₄, filtered, and concentrated to give the product (6.44 g, 28.6 mmol, 97% yield) as a white solid.

MS (ES⁺) C₇H₇Cl₃N₂ requires: 223/225, found: 224/226 [M+H]⁺.

### Step 2: 2,4-Dichloro-5-isopropyl-6-((4-methoxybenzyl)oxy)pyrimidine

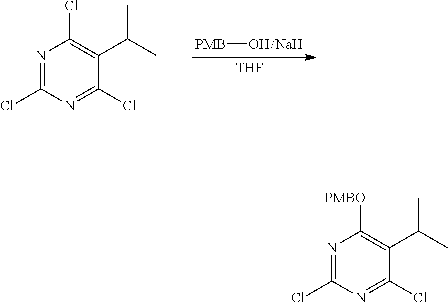

To a stirred solution of the material from the previous step (1 g, 4.43 mmol) in THF (5.0 mL) was added (4-methoxyphenyl)methanol (0.306 g, 2.21 mmol), and the reaction mixture was cooled to −20° C. using a dry ice bath. After 5 min, NaH (0.177 g, 4.43 mmol, 60% in mineral oil) was added and the reaction was allowed to stir and warm to RT overnight. The reaction mixture was diluted with EtOAc (100 mL) and washed with sat. NH₄Cl (3×100 mL). The layers were separated, and the organic layer was washed with sat. NaCl (100 mL), dried over Na₂SO₄, filtered and concentrated under reduced pressure. The residue was purified via silica gel chromatography (0-10% Et₂O in hexanes to give the product (503 mg, 1.537 mmol, 69% yield) as a colorless crystalline solid.

MS (ES⁺) C₁₅H₁₆C₁₂N₂O₂: requires 326, poor ionization, not visible.

¹H NMR (DMSO-d₆) δ: 7.43 (d, J=8.7, 2H), 6.98 (d, J=8.3, 2H), 5.39 (s, 2H), 3.77 (s, 3H), 3.38-3.33 (m, 1H), 1.20 (d, J=6.8, 6H).

### Step 3: 2-Chloro-5-isopropyl-4-((4-methoxybenzyl)oxy)-6-(pyridin-3-yloxy)pyrimidine

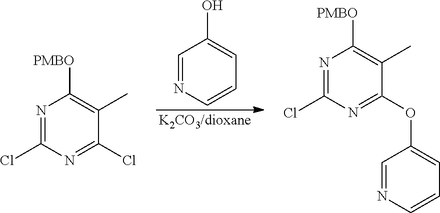

To a solution of the material from the previous step (100 mg, 0.306 mmol) and 3-hydroxypyridine (29.1 mg, 0.306 mmol) in dioxane (3 mL) were added K₂CO₃ (63.4 mg, 0.458 mmol) and the mixture was heated at 80° C. for 48 h. The mixture was acidified with 1N HCl (pH=4-5) and extracted with EtOAc and the organic phase was concentrated. The residue was purified via silica gel chromatography (0-100% EtOAc in hexanes to give the product (58 mg, 0.150 mmol, 49% yield) as a colorless liquid.

MS (ES⁺) C₂₀H₂₀ClN₃O₃ requires: 385, found: 386 [M+H]⁺.

### Step 4: 2-(1H-Imidazol-1-yl)-5-isopropyl-6-(pyridin-3-yloxy)pyrimidin-4-ol (28)

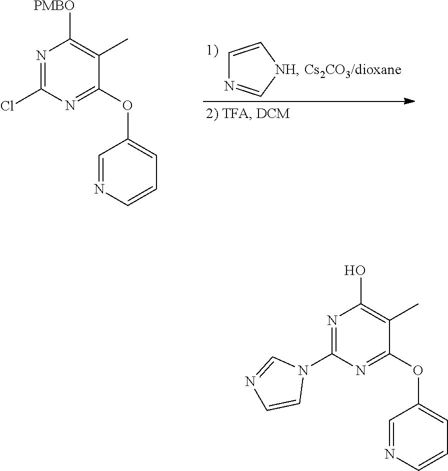

A mixture of the material from the previous step (30 mg, 0.078 mmol), 1H-imidazole (26.5 mg, 0.389 mmol) and Cs₂CO₃ (50.7 mg, 0.156 mmol) in dioxane (0.75 mL) was heated at 120° C. overnight, then the mixture was diluted with DCM and the organic phase was washed with H₂O and concentrated. The residue was dissolved in DCM (0.35 mL) and TFA (0.15 mL) was added. The mixture was left overnight and then evaporated and the residue was purified by mass-triggered preparative HPLC (Mobile phase: A=0.1% TFA/H₂O, B=0.1% TFA/MeCN; Gradient: B=10-40%; 20 min; Column: C18) to give the product 28 (1.5 mg, 2.86 μmol, 3.6% yield) as a bis-TFA salt.

MS (ES⁺) C₁₅H₁₅N₅O₂ requires: 297, found: 298 [M+H]⁺.

¹H NMR (500 MHz, DMSO-d₆) δ: 13.1 (brs, 1H), 8.70 (brs, 1H), 8.58 (d, J=2.7, 1H), 8.52 (d, J=4.7, 1H), 7.8 (d, J=8.3, 1H), 7.65 (s, 1H), 7.57 (d, J=4.7, 0.5H), 7.55 (d, J=4.7, 0.5H), 7.39 (s, 1H), 3.46-3.36 (m, 1H), 1.33 (d, J=7.0, 6H).

A side product was obtained from this reaction which, after purification, was identified as 2,6-di(1H-imidazol-1-yl)-5-isopropylpyrimidin-4-ol (28b) as bis-TFA salt.

MS (ES⁺) C₁₃H₁₄N₆O requires: 270, found: 271 [M+H]⁺.

¹H NMR (600 MHz, DMSO-d₆) δ: 8.89-8.91 (m, 2H), 7.97 (s, 1H), 7.85 (s, 1H), 7.61 (s, 1H), 7.36 (s, 1H), 2.85-2.77 (m, 1H), 1.29 (d, J=6.9, 6H).

### Example 29

### 2-(1H-Imidazol-1-yl)-5-isopropyl-6-(pyridin-3-ylmethoxy)pyrimidin-4-ol

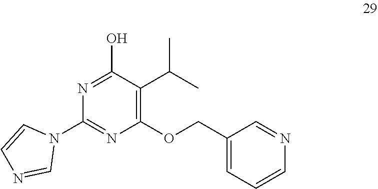 [SMILES: CC(C)c1c(O)nc(-n2ccnc2)nc1OCc1cccnc1]

### Step 1: 2-Chloro-5-isopropyl-4-((4-methoxybenzyl)oxy)-6-(pyridin-3-ylmethoxy)pyrimidine

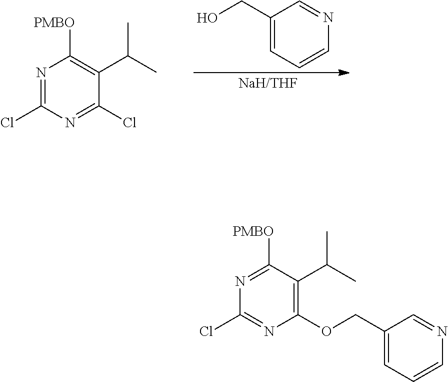

To a solution of 2,4-dichloro-5-isopropyl-6-((4-methoxybenzyl)oxy)pyrimidine (200 mg, 0.611 mmol) and pyridin-3-yl-methanol (59.3 al, 0.611 mmol) in THF (6 mL) under N₂ and cooled in an ice bath was added NaH (60% in mineral oil, 29 mg, 0.733 mmol). The resulting mixture was stirred in the ice bath and allowed to warm to RT overnight. The reaction was quenched with saturated NH₄Cl, concentrated, and partitioned between H₂O and EtOAc. The organic layer was separated, dried over MgSO₄, supported on silica gel, and purified by flash chromatography (0-50% EtOAc in hexanes) to give the product (200 mg, 0.50 mmol, 82% yield).

MS (ES⁺) C₂₁H₂₂ClN₃O₃ requires: 399, found: 400 [M+H]⁺.

### Step 2: 2-(1H-Imidazol-1-yl)-5-isopropyl-6-(pyridin-3-ylmethoxy)pyrimidin-4-ol (29)

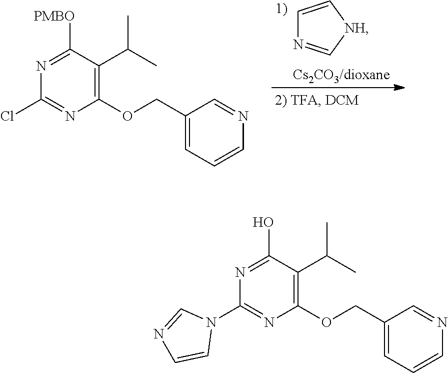

To a solution of the material from the previous step (28 mg, 0.071 mmol) in dioxane (0.5 mL) were added 1H-imidazole (10 mg, 0.143 mmol) and Cs₂CO₃ (46 mg, 0.143 mmol) and the resulting mixture was stirred at 120° C. overnight. The reaction mixture was partitioned between H₂O and DCM. The organic layer was concentrated, dissolved in DCM (0.6 mL) and TFA (0.6 mL) and was stirred at RT for 3 h. The mixture was concentrated and purified by mass-triggered preparative HPLC (Mobile phase: A=0.1% TFA/H₂O, B=0.1% TFA/MeCN; Gradient: B=10-40%; 20 min; Column: C18) and lyophilized to give the product 29 (2 mg, 3.7 μmol, 5% yield) as white solid (bis-TFA salt).

MS (ES⁺) C₁₆H₁₇N₅O₂ requires: 311, found: 312 [M+H]⁺.

¹H NMR (500 MHz, Methanol-d₄) δ: 9.66 (s, 1H), 8.91 (s, 1H), 8.74 (d, J=5.2, 1H), 8.47 (d, J=7.9, 1H), 8.31 (s, 1H), 7.90 (m, 1H), 7.70 (s, 1H), 5.75 (s, 2H), 3.48-3.39 (m, 1H), 1.31 (d, J=7.0, 6H).

### Example 30

### 5-isopropyl-6-(1-methyl-1H-pyrazol-4-yl)-2-(thiazol-5-yl)pyrimidin-4-ol

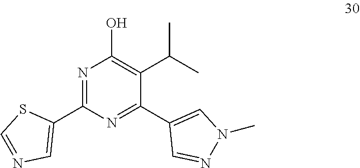 [SMILES: CC(C)c1c(O)nc(-c2cncs2)nc1-c1cnn(C)c1]

### Step 1: 2-Chloro-5-isopropyl-4-((4-methoxybenzyl)oxy)-6-(1-methyl-1H-pyrazol-4-yl)pyrimidine

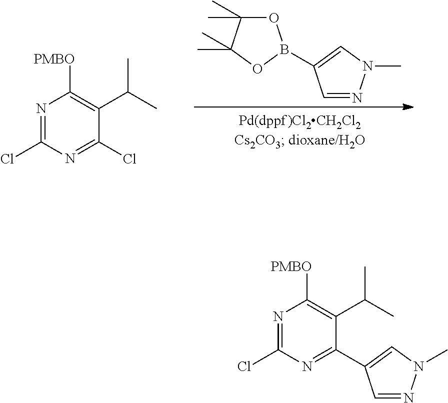

To a 4 mL vial equipped with a stir bar, 2,4-dichloro-5-isopropyl-6-((4-methoxy-benzyl)oxy)pyrimidine (Example 28, Step 2) (100 mg, 0.306 mmol), 1-methyl-4-(4,4,5,5-tetramethyl-1,3,2-dioxaborolan-2-yl)-1H-pyrazole (69.9 mg, 0.336 mmol), PdCl₂(dppf)-CH₂Cl₂ adduct (37.4 mg, 0.046 mmol), and Cs₂CO₃ (299 mg, 0.917 mmol) were added followed by degassed dioxane (1.5 mL) and H₂O (0.017 mL, 0.917 mmol). The reaction mixture was heated to 90° C. and allowed to stir for 15 h. The reaction mixture was diluted with EtOAc (30 mL) and washed with H₂O (3×30 mL). The layers were separated, and the organic layer was washed with sat. NaCl (30 mL), dried over Na₂SO₄ and concentrated under reduced pressure. The residue was purified via silica gel chromatography (0-40% EtOAc in hexanes to give the product (64.1 mg, 0.172 mmol, 56% yield) as a white solid.

MS (ES⁺) C₁₉H₂₁ClN₄O₂ requires: 372, found: 373 [M+H]⁺.

### Step 2: 5-(5-Isopropyl-4-((4-methoxybenzyl)oxy)-6-(1-methyl-1H-pyrazol-4-yl)pyrimidin-2-yl)-thiazole

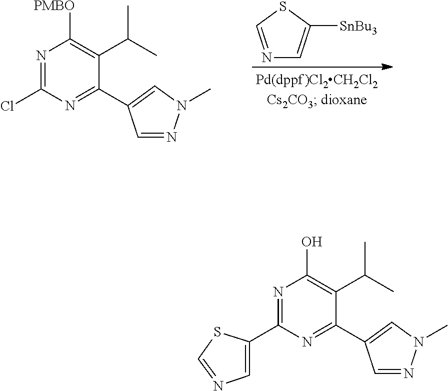

To a stirred solution of the material from the previous step (30 mg, 0.080 mmol) in degassed dioxane (0.8 mL) were added 5-(tributylstannyl)thiazole (36.1 mg, 0.097 mmol) and PdCl₂(dppf)-CH₂Cl₂ adduct (13.1 mg, 0.016 mmol), and the reaction mixture was heated to 80° C. After 48 h, the reaction mixture was diluted with EtOAc (30 mL) and washed with H₂O (3×30 mL). The layers were separated, and the organic layer was washed with sat. NaCl (30 mL), dried over Na₂SO₄, and concentrated under reduced pressure. The residue was purified via silica gel chromatography (0-100% EtOAc in hexanes to give the product (9.7 mg, 0.023 mmol, 29% yield).

MS (ES⁺) C₂₂H₂₃N₅O₂S requires: 421, found: 422 [M+H]⁺.

### Step 3: 5-Isopropyl-6-(1-methyl-1H-pyrazol-4-yl)-2-(thiazol-5-yl)pyrimidin-4-ol (30)

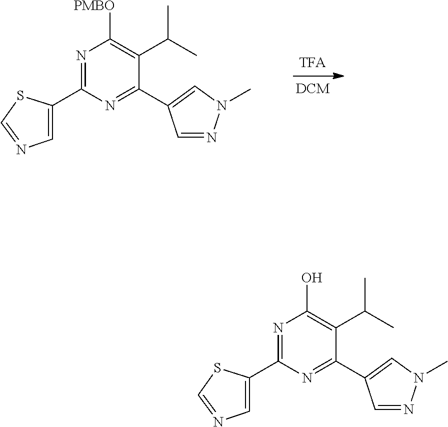

To a stirred solution of the material from the previous step (9.7 mg, 0.023 mmol) in DCM (0.23 mL) was added TFA (53 μl, 0.69 mmol), and the mixture was allowed to stir for 1 h. The volatiles were removed under reduced pressure. The residue was purified by mass-triggered preparative HPLC (Mobile phase: A=0.1% TFA/H₂O, B=0.1% TFA/MeCN; Gradient: B=20-60%; 12 min; Column: C18) to give the product 30 (1.5 mg, 4.98 μmol, 21% yield).

MS (ES⁺) C₁₄H₁₅N₅OS requires: 301, found: 302 [M+H]⁺.

¹H NMR (500 MHz, DMSO-d₆) δ: 12.52 (brs, 1H), 9.25 (s, 1H), 8.42 (s, 1H), 8.17 (s, 1H), 8.13 (s, 1H), 3.90 (s, 3H), 3.21-3.16 (m, 1H), 1.34 (d, J=6.7, 6H).

### Example 31

### 6-methyl-2-(1-methyl-1H-imidazol-5-yl)-5-(propan-2-yl)pyrimidin-4-ol

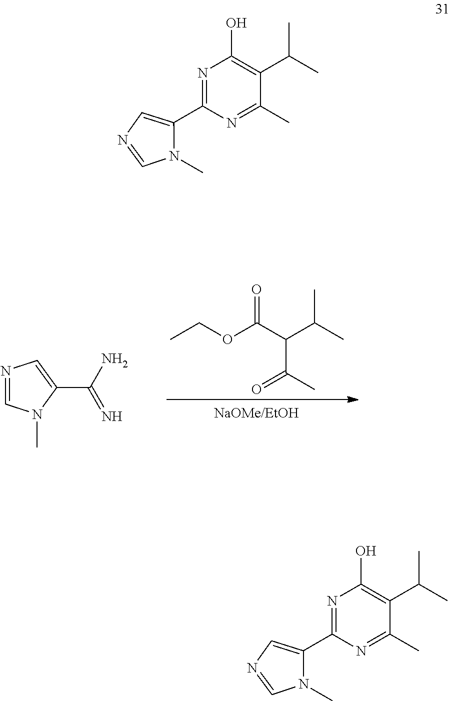

To a solution of 1-methyl-1H-imidazole-5-carboximidamide trifluoroacetate (50 mg, 0.210 mmol) (prepared by the method of Example 19, Step 1) in EtOH (0.5 mL) were added NaOMe in EtOH (272 mg, 0.840 mmol, 21 wt. %) and ethyl 2-acetyl-3-methylbutanoate (54 mg, 0.315 mmol). The resulting mixture was stirred at 80° C. overnight. The solution was quenched with 1M HCl and concentrated. The residue was purified by mass-triggered preparative HPLC (Mobile phase: A=0.1% TFA/H₂O, B=0.1% TFA/MeCN; Gradient: B=40-80%; 12 min; Column: C18) to give the product 31 (0.6 mg, 1.7 μmol, 0.8% yield) as an orange solid (TFA salt).

MS (ES⁺) C₁₂H₁₆N₄O requires: 232, found: 233 [M+H]⁺.

¹H NMR (600 MHz, DMSO-d₆) δ: 12.49 (brs, 1H), 8.50 (brs, 1H), 8.04 (s, 1H), 4.03 (s, 3H), 3.16-3.01 (m, 1H), 2.34 (s, 3H), 1.26 (d, J=7.0, 6H).

### Example 32

### 5-cyclopropyl-2-(thiazol-5-yl)pyrimidin-4-ol

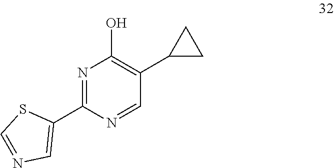 [SMILES: Oc1nc(-c2cncs2)ncc1C1CC1]

### Step 1: 5-Bromo-2-chloro-4-((4-methoxybenzyl)oxy)pyrimidine

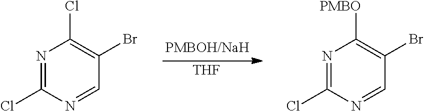

To a solution of (4-methoxyphenyl)methanol (152 mg, 1.097 mmol) in THF (3 mL) at −15° C. were added 5-bromo-2,4-dichloropyrimidine (500 mg, 2.194 mmol) and NaH in 60% mineral oil (53 mg, 1.317 mmol). and the mixture was stirred for 2 h, allowing to warm to RT. Additional NaH in 60% mineral oil (53 mg, 1.317 mmol) was added and the reaction was stirred an additional overnight then quenched with MeOH and saturated NaCl was added. The aqueous phase was extracted with EtOAc (3×), the combined organic layers were concentrated under reduced pressure. The residue was purified via silica gel chromatography (0-40% EtOAc in hexanes to give the product (164 mg, 0.498 mmol, 45% yield) as a white solid.

MS (ES⁺) C₁₂H₁₀BrClN₂O₂ requires: 329, found: 330 [M+H]⁺.

### Step 2: 2-Chloro-5-cyclopropyl-4-((4-methoxybenzyl)oxy)pyrimidine

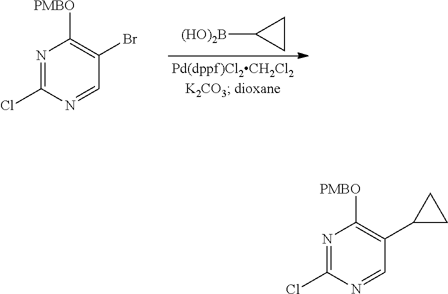

A mixture of the material from the previous step (15 mg, 0.046 mmol), cyclopropyl-boronic acid (7.8 mg, 0.091 mmol), PdCl₂(dppf)-CH₂Cl₂ adduct (5.5 mg, 6.83 μmol), K₂CO₃ (19 mg, 0.137 mmol) in dioxane (0.45 mL) was degassed under N₂ and heated at 100° C. overnight. The solution was directly purified via silica gel chromatography (0-30% EtOAc in hexanes) to give the product (7 mg, 0.024 mmol, 53% yield) as a colorless amorphous material.

MS (ES⁺) C₁₅H₁₅ClN₂O₂ requires: 219, found: 313 [M+H+Na]⁺.

### Step 3: 5-Cyclopropyl-2-(thiazol-5-yl)pyrimidin-4-ol (32)

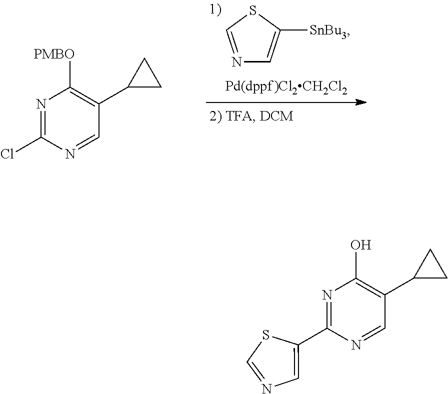

A mixture of the material from the previous step (7 mg, 0.024 mmol), 5-(tributyl-stannyl)thiazole (14 μl, 0.043 mmol), and PdCl₂(dppf)-CH₂Cl₂ adduct (3.9 mg, 4.82 μmol) was degassed and heated at 120° C. for 2 h, then directly purified via silica gel chromatography (0-30% EtOAc in hexanes to give 5-(5-cyclopropyl-4-((4-methoxybenzyl)oxy)pyrimidin-2-yl)thiazole (5 mg, 0.015 mmol, 61% yield) as a yellow liquid. This material was directly dissolved in DCM (0.35 mL) and TFA (0.15 mL) and the mixture was stirred at RT for 2 h and then evaporated. The residue was purified by mass-triggered preparative HPLC (Mobile phase: A=0.1% TFA/H₂O, B=0.1% TFA/MeCN; Gradient: B=10-40%; 20 min; Column: C18) to give the product 32 (1.2 mg, 5.47 μmol, 22% yield) as a pale yellow solid.

MS (ES⁺) C₁₀H₉N₃OS requires: 219, found: 220 [M+H]⁺.

¹H NMR (600 MHz, DMSO-d₆) δ: 13.05 (brs, 1H), 9.25 (s, 1H), 8.80 (s, 1H), 7.63 (s, 1H), 1.87-1.81 (m, 1H), 0.88-0.82 (m, 2H), 0.79-0.74 (m, 2H).

### Example 33

### 2-(1,3-Thiazol-5-yl)-5-(trifluoromethyl)pyrimidin-4-ol

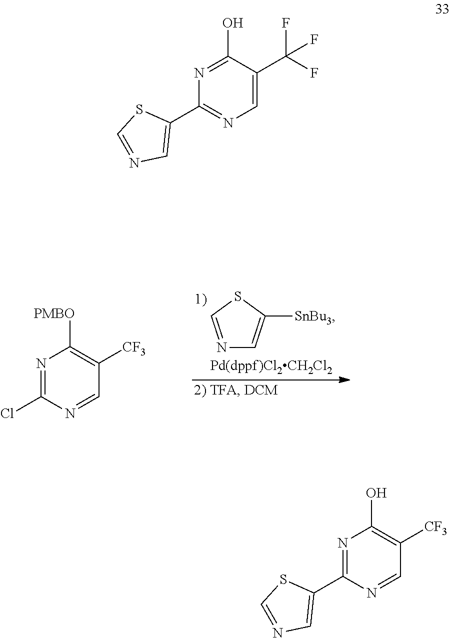

A mixture of 2-chloro-4-((4-methoxybenzyl)oxy)-5-(trifluoromethyl)pyrimidine (prepared by the method of Example 32, Step 1, using 2,4-dichloro-5-(trifluoromethyl)-pyrimidine as starting material) (30 mg, 0.094 mmol), 5-(tributylstannyl)thiazole (0.037 mL, 0.113 mmol), PdCl₂(dppf)-CH₂Cl₂ adduct (15 mg, 0.019 mmol) was degassed under N₂ and heated at 100° C. for 3 h. The residue was directly purified via silica gel chromatography (0-40% EtOAc in hexanes to give 5-(4-((4-methoxybenzyl)oxy)-5-(trifluoromethyl)pyrimidin-2-yl)thiazole (30 mg, 0.082 mmol, 87% yield) as a yellow solid. DCM (0.3 mL) and TFA (0.15 mL) was added and stirred for 1 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 12 min; Column: C18) to give the product 33 as a white solid.

MS (ES⁺) C₈H₄F₃N₃OS requires: 247, found: 248 [M+H]⁺.

¹H NMR (600 MHz, DMSO-d₆) δ: 13.87 (brs, 1H), 9.39 (s, 1H), 8.92 (s, 1H), 8.47 (s, 1H).

The activity of the compounds in Examples 1-(#) as KDM5 (inhibitors/modulators/etc.) is illustrated in the following assay(s). The other compounds listed above, which have not yet been made and/or tested, are predicted to have activity in these assay(s) as well.

TABLE 1
Synthesized examples

| Example | Scheme | IUPAC Name | Structure |
|---|---|---|---|
| 1 | 1 | 2-(1H-Imidazol-1-yl)-5- (propan-2-yl)-6-[1-(propan-2- yl)-1H-pyrazol-4-yl]- pyrimidin-4-ol | 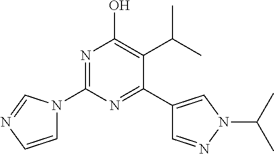 [SMILES: CC(C)c1c(O)nc(-n2ccnc2)nc1-c1cnn(C(C)C)c1] |
| 2 | 2 | 6-Chloro-5-(propan-2-yl)-2- [2-(pyridin-2-yl)-1H- imidazol-1-yl]pyrimidin-4-ol | 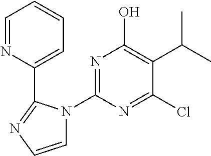 [SMILES: CC(C)c1c(O)nc(-n2ccnc2-c2ccccn2)nc1Cl] |
| 3 | 3 | 5-(Propan-2-yl)-6-[1-(propan- 2-yl)-1H-pyrazol-4-yl)-2-[2- (pyridin-2-yl)-1H-imidazol-1- yl]pyrimidin-4-ol | 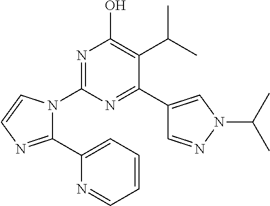 [SMILES: CC(C)c1c(O)nc(-n2ccnc2-c2ccccn2)nc1-c1cnn(C(C)C)c1] |
| 4 | 4 | 2-(2-Methyl-1H-imidazol-1- yl)-5-(propan-2-yl)-6-[1- (propan-2-yl)-1H-pyrazol-4- yl]pyrimidin-4-ol | 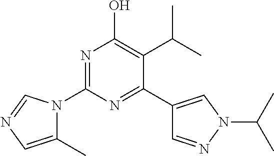 [SMILES: Cc1cncn1-c1nc(O)c(C(C)C)c(-c2cnn(C(C)C)c2)n1] |
| 5 | 5 | 2-{4-[6-Hydroxy-2-(1H- imidazol-1-yl)-5-(propan-2- yl)pyrimidin-4-yl]-1H- pyrazol-1-yl}acetic acid | 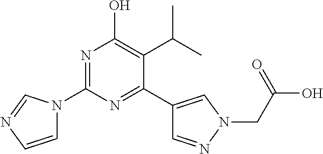 [SMILES: CC(C)c1c(O)nc(-n2ccnc2)nc1-c1cnn(CC(=O)O)c1] |
| 6 | 6 | 2-{4-[6-Hydroxy-2-(1H- Imidazol-1-yl)-5-(propan-2- yl)pyrimidin-4-yl]-1H- pyrazol-1-yl}-N-(2- (pyrrolidin-1-yl)ethyl]- acetamide | 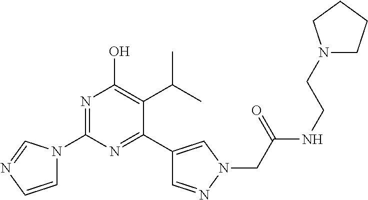 [SMILES: CC(C)c1c(O)nc(-n2ccnc2)nc1-c1cnn(CC(=O)NCCN2CCCC2)c1] |
| 7 | 7 | 2-{4-[6-Hydroxy-2-(1H- imidazol-1-yl)-5-(propan-2- yl)pyrimidin-4-yl]-1H- pyrazol-1-yl}acetamide | 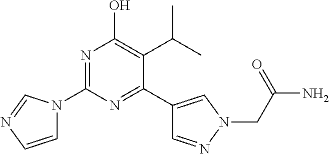 [SMILES: CC(C)c1c(O)nc(-n2ccnc2)nc1-c1cnn(CC(N)=O)c1] |
| 8 | 8 | N-[2-(Dimethylamino)ethyl]- 5-[6-hydroxy-2-(1H-imidazol- 1-yl)-5-(propan-2-yl)- pyrimidin-4-yl]pyridine-2- carboxamide | 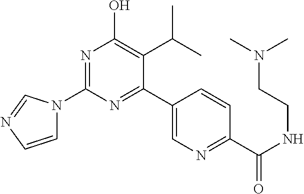 [SMILES: CC(C)c1c(O)nc(-n2ccnc2)nc1-c1ccc(C(=O)NCCN(C)C)nc1] |
| 9 | 9 | 5-[6-Hydroxy-2-(1H- imidazol-1-yl)-5-(propan-2- yl)pyrimidin-4-yl]pyridine-2- carboxamide | 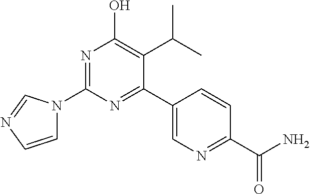 [SMILES: CC(C)c1c(O)nc(-n2ccnc2)nc1-c1ccc(C(N)=O)nc1] |
| 10a | 10 | 2-(1H-imidazol-1-yl)-5- (propan-2-yl)-6-[2-(pyridin-3- yl)ethynyl]pyrimidin-4-ol | 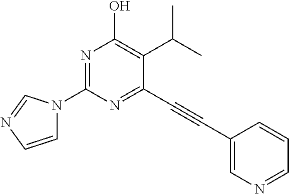 [SMILES: CC(C)c1c(O)nc(-n2ccnc2)nc1C#Cc1cccnc1] |
| 10b | 10 | 2-(1H-Imidazol-1-yl)-5- (propan-2-yl)-6-(2-(pyridin-3- yl)ethyl]pyrimidin-4-ol | 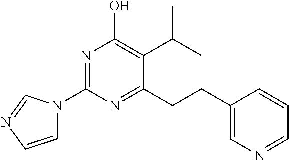 [SMILES: CC(C)c1c(O)nc(-n2ccnc2)nc1CCc1cccnc1] |
| 11 | 11 | 2-(1H-Imidazol-1-yl)-6-[2-(1- methylpiperidin-4-yl)ethoxy]- 5-(propan-2-yl)pyrimidin-4-ol | 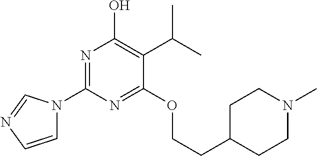 [SMILES: CC(C)c1c(O)nc(-n2ccnc2)nc1OCCC1CCN(C)CC1] |
| 12 | 12 | 2-(1H-Imidazol-1-yl)-6- methoxy-5-(propan-2-yl)- pyrimidin-4-ol | 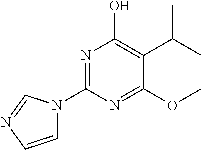 [SMILES: COc1nc(-n2ccnc2)nc(O)c1C(C)C] |
| 13 | 13 | 2-(1H-Imidazol-1-yl)-5- (propan-2-yl)-6-{2-[1-(4,4,4- trifluorobutyl)piperidin-4-yl]- ethoxy}pyrimidin-4-ol | 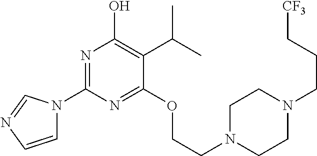 [SMILES: CC(C)c1c(O)nc(-n2ccnc2)nc1OCCN1CCN(CCCC(F)(F)F)CC1] |
| 14 | 14 | 3-Hydroxy-1-[4-(2-{[6- hydroxy-2-(1H-imidazol-1- yl)-5-(propan-2-yl)pyrimidin- 4-yl]oxy}ethyl)piperidin-1- yl]propan-1-one | 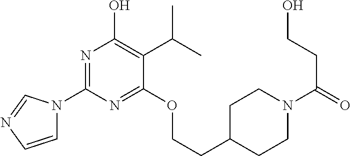 [SMILES: CC(C)c1c(O)nc(-n2ccnc2)nc1OCCC1CCN(C(=O)CCO)CC1] |
| 15 | 15 | 2-{[6-Hydroxy-2-(1H- imidazol-1-yl)-5-(propan-2- yl)pyrimidin-4-yl]oxy}acetic acid | 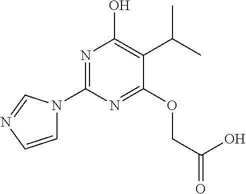 [SMILES: CC(C)c1c(O)nc(-n2ccnc2)nc1OCC(=O)O] |
| 16 | 16 | 2-{[6-Hydroxy-2-(1H- imidazol-1-yl)-5-(propan-2- yl)pyrimidin-4-yl]oxy}-1-(4- methylpiperazin-1-yl)ethan-1- one | 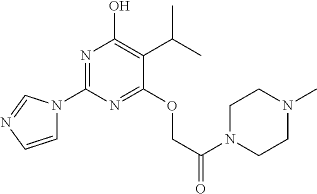 [SMILES: CC(C)c1c(O)nc(-n2ccnc2)nc1OCC(=O)N1CCN(C)CC1] |
| 17 | 17 | 2-{[6-Hydroxy-2-(1H- imidazol-1-yl)-5-(propan-2- yl)pyrimidin-4-yl]oxy}-N- methanesulfonylacetamide | 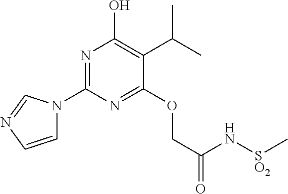 [SMILES: CC(C)c1c(O)nc(-n2ccnc2)nc1OCC(=O)NS(C)(=O)=O] |
| 18 | 18 | 2-(1H-Imidazol-1-yl)-5- (propan-2-yl)-6-[(pyridin-3- ylmethyl)amino]pyrimidin-4- ol | 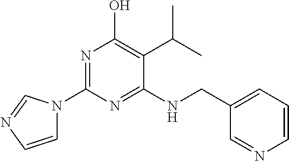 [SMILES: CC(C)c1c(O)nc(-n2ccnc2)nc1NCc1cccnc1] |
| 19 | 19 | 5-(Propan-2-yl)-6-(pyridin-3- ylmethoxy)-2-(1,3-thiazol-5- yl)pyrimidin-4-ol | 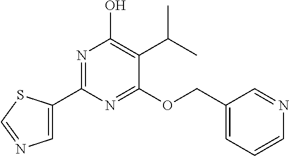 [SMILES: CC(C)c1c(O)nc(-c2cncs2)nc1OCc1cccnc1] |
| 20 | 20 | 6-(1-Benzyl-1H-pyrazol-4- yl)-5-(propan-2-yl)-2-(1,3- thiazol-5-yl)pyrimidin-4-ol | 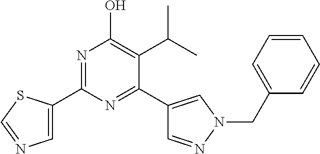 [SMILES: CC(C)c1c(O)nc(-c2cncs2)nc1-c1cnn(Cc2ccccc2)c1] |
| 21 | 21 | 2-(1H-Imidazol-4-yl)-6- methyl-5-(propan-2-yl)- pyrimidin-4-ol | 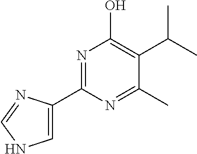 [SMILES: Cc1nc(-c2c[nH]cn2)nc(O)c1C(C)C] |
| 22 | 22 | 2-(1H-Imidazol-1-yl)-6- methyl-5-(propan-2-yl)- pyrimidin-4-ol | 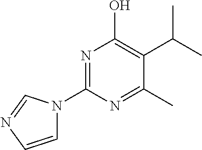 [SMILES: Cc1nc(-n2ccnc2)nc(O)c1C(C)C] |
| 23 | 23 | 2-(2-Amino-1H-imidazol-1- yl)-6-methyl-5-(propan-2- yl)pyrimidin-4-ol | 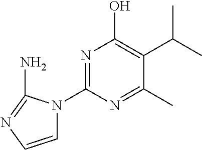 [SMILES: Cc1nc(-n2ccnc2N)nc(O)c1C(C)C] |
| 24a | 24 | 2-[5-(Hydroxymethyl)-1H- imidazol-1-yl]-6-methyl-5- (propan-2-yl)pyrimidin-4-ol | 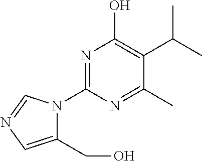 [SMILES: Cc1nc(-n2cncc2CO)nc(O)c1C(C)C] |
| 24b | 24 | 2-[4-(Hydroxymethyl)-1H- imidazol-1-yl]-6-methyl-5- (propan-2-yl)pyrimidin-4-ol | 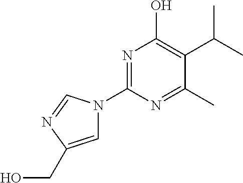 [SMILES: Cc1nc(-n2cnc(CO)c2)nc(O)c1C(C)C] |
| 25 | 25 | 2-[2-(Hydroxymethyl)-1H- imidazol-1-yl]-6-methyl-5- (propan-2-yl)pyrimidin-4-ol | 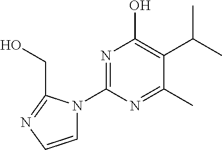 [SMILES: Cc1nc(-n2ccnc2CO)nc(O)c1C(C)C] |
| 26 | 26 | 6-Methyl-5-(propan-2-yl)-2- (1H-pyrazol-4-yl)pyrimidin- 4-ol | 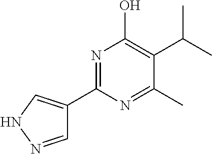 [SMILES: Cc1nc(-c2cn[nH]c2)nc(O)c1C(C)C] |
| 27 | 27 | 2-[1-(2-Hydroxyethyl)-1H- imidazol-5-yl]-6-methyl-5- (propan-2-yl)pyrimidin-4-ol | 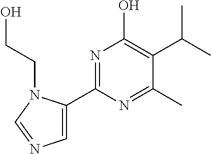 [SMILES: Cc1nc(-c2cncn2CCO)nc(O)c1C(C)C] |
| 28a | 28 | 2-(1H-Imidazol-1-yl)-5- (propan-2-yl)-6-(pyridin-3- yloxy)pyrimidin-4-ol | 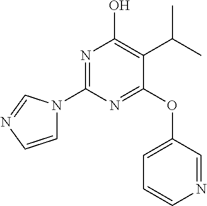 [SMILES: CC(C)c1c(O)nc(-n2ccnc2)nc1Oc1cccnc1] |
| 28b | 28 | 2,6-Bis(1H-imidazol-1-yl)-5- (propan-2-yl)pyrimidin-4-ol | 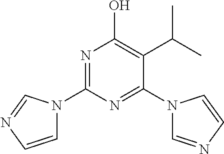 [SMILES: CC(C)c1c(O)nc(-n2ccnc2)nc1-n1ccnc1] |
| 29 | 29 | 2-(1H-Imidazol-1-yl)-5- (propan-2-yl)-6-(pyridin-3-yl- methoxy)pyrimidin-4-ol | 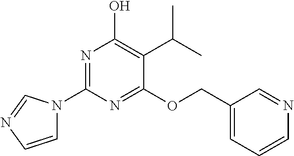 [SMILES: CC(C)c1c(O)nc(-n2ccnc2)nc1OCc1cccnc1] |
| 30 | 30 | 6-(1-Methyl-1H-pyrazol-4- yl)-5-(propan-2-yl)-2-(1,3- thiazol-5-yl)pyrimidin-4-ol | 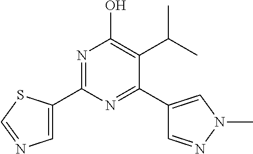 [SMILES: CC(C)c1c(O)nc(-c2cncs2)nc1-c1cnn(C)c1] |
| 31 | 31 | 6-Methyl-2-(1-methyl-1H- imidazol-5-yl)-5-(propan-2- yl)pyrimidin-4-ol | 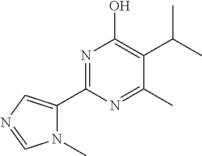 [SMILES: Cc1nc(-c2cncn2C)nc(O)c1C(C)C] |
| 32 | 32 | 5-Cyclopropyl-2-(1,3-thiazol- 5-yl)pyrimidin-4-ol | 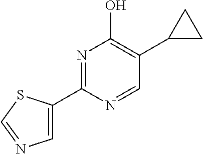 [SMILES: Oc1nc(-c2cncs2)ncc1C1CC1] |
| 33 | 33 | 2-(1,3-Thiazol-5-yl)-5-(tri- fluoromethyl)pyrimidin-4-ol | 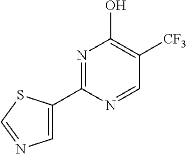 [SMILES: Oc1nc(-c2cncs2)ncc1C(F)(F)F] |
| 41 | 1 | 6-[3-(Aminomethyl)phenyl]- 2-(1H-imidazol-1-yl)-5- (propan-2-yl)pyrimidin-4-ol | 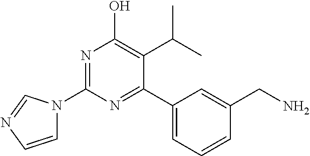 [SMILES: CC(C)c1c(O)nc(-n2ccnc2)nc1-c1cccc(CN)c1] |
| 42 | 1 | 6-[4-(Aminomethyl)phenyl]- 2-(1H-imidazol-1-yl)-5- (propan-2-yl)pyrimidin-4-ol | 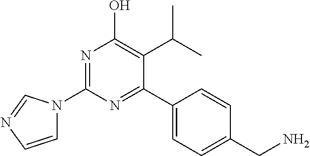 [SMILES: CC(C)c1c(O)nc(-n2ccnc2)nc1-c1ccc(CN)cc1] |
| 43 | 1 | N-({4-[6-Hydroxy-2-(1H- imidazol-1-yl)-5-(propan-2- yl)pyrimidin-4-yl]phenyl}- methyl)acetamide | 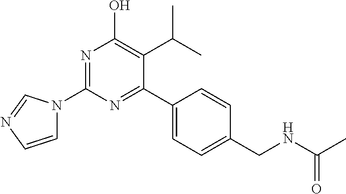 [SMILES: CC(=O)NCc1ccc(-c2nc(-n3ccnc3)nc(O)c2C(C)C)cc1] |
| 44 | 1 | 3-{4-[6-Hydroxy-2-(1H- imidazol-1-yl)-5-(propan-2- yl)pyrimidin-4-yl]-1H- pyrazol-1-yl}propanenitrile | 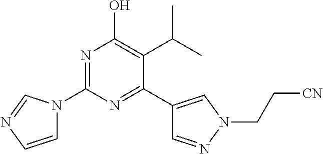 [SMILES: CC(C)c1c(O)nc(-n2ccnc2)nc1-c1cnn(CCC#N)c1] |
| 45 | 1 | 2-(1H-Imidazol-1-yl)-5- (propan-2-yl)-6-[1-(pyridin-3- ylmethyl)-1H-pyrazol-4-yl]- pyrimidin-4-ol | 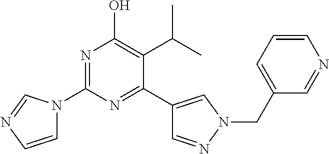 [SMILES: CC(C)c1c(O)nc(-n2ccnc2)nc1-c1cnn(Cc2cccnc2)c1] |
| 46 | 1 | 2-(1H-Imidazol-1-yl)-5- (propan-2-yl)-6-(1H-pyrazol- 4-yl)pyrimidin-4-ol | 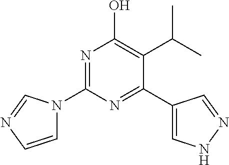 [SMILES: CC(C)c1c(O)nc(-n2ccnc2)nc1-c1cn[nH]c1] |
| 47 | 1 | 6-(1-Cyclopropyl-1H-pyrazol- 4-yl)-2-(1H-imidazol-1-yl)-5- (propan-2-yl)pyrimidin-4-ol | 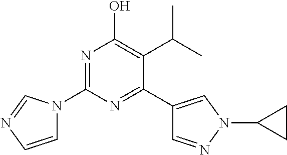 [SMILES: CC(C)c1c(O)nc(-n2ccnc2)nc1-c1cnn(C2CC2)c1] |
| 48 | 1 | N-({4-[6-Hydroxy-2-(1H- imidazol-1-yl)-5-(propan-2- yl)pyrimidin-4-yl]phenyl}- methyl)methanesulfonamide | 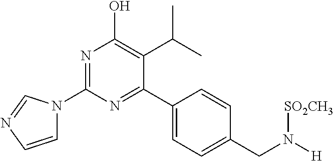 [SMILES: CC(C)c1c(O)nc(-n2ccnc2)nc1-c1ccc(CNS(C)(=O)=O)cc1] |
| 49 | 1 | 6-(1-Cyclobutyl-1H-pyrazol- 4-yl)-2-(1H 1H-imidazol-1- yl)-5-(propan-2-yl)pyrimidin- 4-ol | 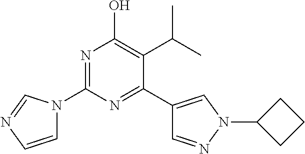 [SMILES: CC(C)c1c(O)nc(-n2ccnc2)nc1-c1cnn(C2CCC2)c1] |
| 50 | 1 | N-{3-[6-Hydroxy-2-(1H- imidazol-1-yl)-5-(propan-2- yl)pyrimidin-4-yl]phenyl}- acetamide | 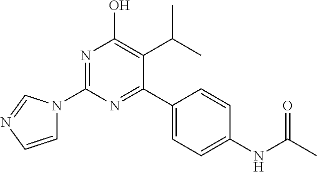 [SMILES: CC(=O)Nc1ccc(-c2nc(-n3ccnc3)nc(O)c2C(C)C)cc1] |
| 51 | 1 | 2-(1H-Imidazol-1-yl)-6-[1-(2- methoxyethyl)-1H-pyrazol-4- yl]-5-(propan-2-yl)pyrimidin- 4-ol | 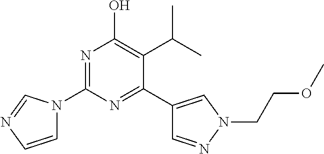 [SMILES: COCCn1cc(-c2nc(-n3ccnc3)nc(O)c2C(C)C)cn1] |
| 52 | 1 | 6-[1-(Cyclopropylmethyl)- 1H-pyrazol-4-yl]-2-(1H- imidazol-1-yl)-5-(propan-2- yl)pyrimidin-4-ol | 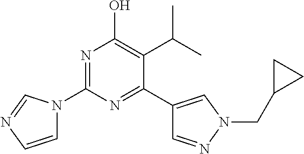 [SMILES: CC(C)c1c(O)nc(-n2ccnc2)nc1-c1cnn(CC2CC2)c1] |
| 53 | 1 | 2-(1H-Imidazol-1-yl)-5- (propan-2-yl)-6-[1-(pyridin-4- ylmethyl)-1H-pyrazol-4-yl]- pyrimidin-4-ol | 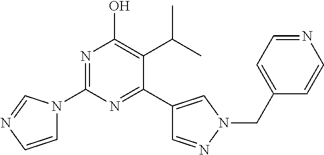 [SMILES: CC(C)c1c(O)nc(-n2ccnc2)nc1-c1cnn(Cc2ccncc2)c1] |
| 54 | 1 | 6-(3-Amino-4-methylphenyl)- 2-(1H-imidazol-1-yl)-5- (propan-2-yl)pyrimidin-4-ol | 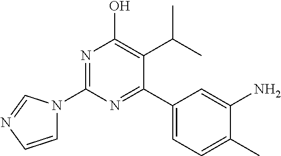 [SMILES: Cc1ccc(-c2nc(-n3ccnc3)nc(O)c2C(C)C)cc1N] |
| 55 | 1 | 2-(1H-Imidazol-1-yl)-6-(1- methyl-1H-pyrazol-4-yl)-5- (propan-2-yl)pyrimidin-4-ol | 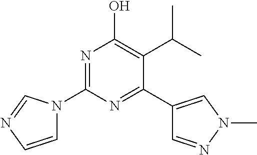 [SMILES: CC(C)c1c(O)nc(-n2ccnc2)nc1-c1cnn(C)c1] |
| 56 | 1 | 2-(1H-Imidazol-1-yl)-5- (propan-2-yl)-6-(1-propyl-1H- pyrazol-4-yl)pyrimidin-4-ol | 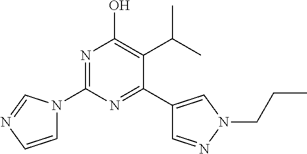 [SMILES: CCCn1cc(-c2nc(-n3ccnc3)nc(O)c2C(C)C)cn1] |
| 57 | 1 | 3-[6-Hydroxy-2-(1H- imidazol-1-yl)-5-(propan-2- yl)pyrimidin-4-yl]-N-methyl- benzamide | 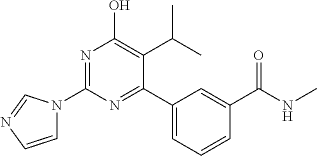 [SMILES: CNC(=O)c1cccc(-c2nc(-n3ccnc3)nc(O)c2C(C)C)c1] |
| 58 | 1 | 6-[4-(Hydroxymethyl)- phenyl]-2-(1H-imidazol-1-yl)- 5-(propan-2-yl)pyrimidin-4-ol | 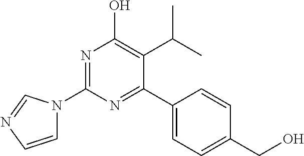 [SMILES: CC(C)c1c(O)nc(-n2ccnc2)nc1-c1ccc(CO)cc1] |
| 59 | 1 | 2-(1H-Imidazol-1-yl)-6-[1- (oxan-4-yl)-1H-pyrazol-4-yl]- 5-(propan-2-yl)pyrimidin-4-ol | 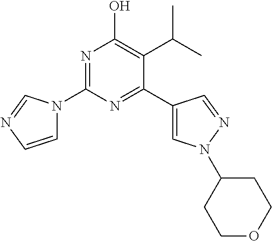 [SMILES: CC(C)c1c(O)nc(-n2ccnc2)nc1-c1cnn(C2CCOCC2)c1] |
| 60 | 1 | 2-(1H-Imidazol-1-yl)-6-(5- methyl-1H-pyrazol-4-yl)-5- (propan-2-yl)pyrimidin-4-ol | 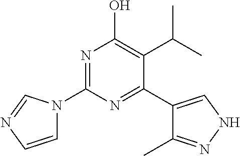 [SMILES: Cc1n[nH]cc1-c1nc(-n2ccnc2)nc(O)c1C(C)C] |
| 61 | 1 | 4-[6-Hydroxy-2-(1H- imidazol-1-yl)-5-(propan-2- yl)pyrimidin-4-yl]-N,N- dimethylbenzene-1- sulfonamide | 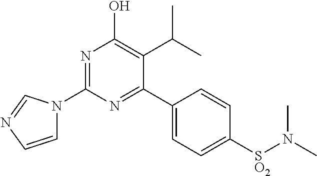 [SMILES: CC(C)c1c(O)nc(-n2ccnc2)nc1-c1ccc(S(=O)(=O)N(C)C)cc1] |
| 62 | 1 | 2-(1H-Imidazol-1-yl)-6-(6- methoxypyridin-3-yl)-5- (propan-2-yl)pyrimidin-4-ol | 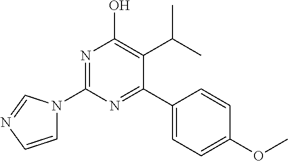 [SMILES: COc1ccc(-c2nc(-n3ccnc3)nc(O)c2C(C)C)cc1] |
| 63 | 1 | 6-(1-Benzyl-1H-pyrazol-4- yl)-2-(1H-imidazol-1-yl)-5- (propan-2-yl)pyrimidin-4-ol | 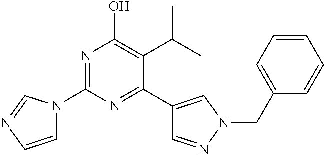 [SMILES: CC(C)c1c(O)nc(-n2ccnc2)nc1-c1cnn(Cc2ccccc2)c1] |
| 64 | 1 | 2-(1H-Imidazol-1-yl)-5- (propan-2-yl)-6-[1-(pyridin-2- ylmethyl)-1H-pyrazol-4-yl]- pyrimidin-4-ol | 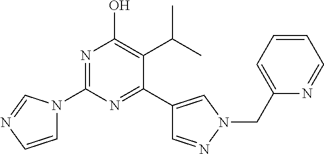 [SMILES: CC(C)c1c(O)nc(-n2ccnc2)nc1-c1cnn(Cc2ccccn2)c1] |
| 65 | 1 | 2-(1H-Imidazol-1-yl)-6-[1-(2- methylpropyl)-1H-pyrazol-4- yl]-5-(propan-2-yl)pyrimidin- 4-ol | 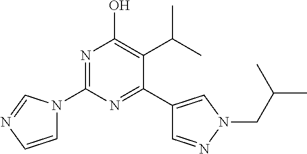 [SMILES: CC(C)Cn1cc(-c2nc(-n3ccnc3)nc(O)c2C(C)C)cn1] |
| 66 | 1 | 6-(1-Ethyl-1H-pyrazol-4-yl)- 2-(1H-imidazol-1-yl)-5- (propan-2-yl)pyrimidin-4-ol | 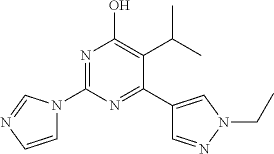 [SMILES: CCn1cc(-c2nc(-n3ccnc3)nc(O)c2C(C)C)cn1] |
| 67 | 1 | 6-(1-Cyclopentyl-1H-pyrazol- 4-yl)-2-(1H-imidazol-1-yl)-5- (propan-2-yl)pyrimidin-4-ol | 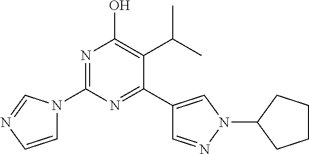 [SMILES: CC(C)c1c(O)nc(-n2ccnc2)nc1-c1cnn(C2CCCC2)c1] |
| 68 | 1 | 2-(1H-Imidazol-1-yl)-6-{1-[2- (morpholin-4-yl)ethyl]-1H- pyrazol-4-yl}-5-(propan-2- yl)pyrimidin-4-ol | 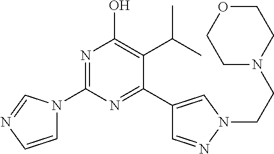 [SMILES: CC(C)c1c(O)nc(-n2ccnc2)nc1-c1cnn(CCN2CCOCC2)c1] |
| 69 | 1 | 2-(1H-Imidazol-1-yl)-6-(4- methanesulfonylphenyl)-5- (propan-2-yl)pyrimidin-4-ol | 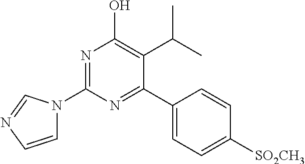 [SMILES: CC(C)c1c(O)nc(-n2ccnc2)nc1-c1ccc(S(C)(=O)=O)cc1] |
| 70 | 1 | 2-(1H-Imidazol-1-yl)-5- (propan-2-yl)-6-(pyridin-3- yl)pyrimidin-4-ol | 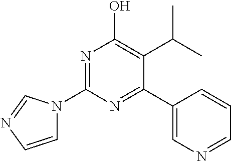 [SMILES: CC(C)c1c(O)nc(-n2ccnc2)nc1-c1cccnc1] |
| 71 | 1 | 2-(1H-Imidazol-1-yl)-6-(1- phenyl-1H-pyrazol-4-yl)-5- (propan-2-yl)pyrimidin-4-ol | 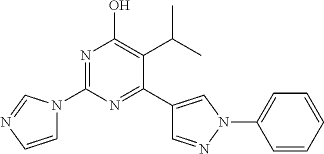 [SMILES: CC(C)c1c(O)nc(-n2ccnc2)nc1-c1cnn(-c2ccccc2)c1] |
| 72 | 1 | 2-(1H-Imidazol-1-yl)-6-(3- methanesulfonylphenyl)-5- (propan-2-yl)pyrimidin-4-ol | 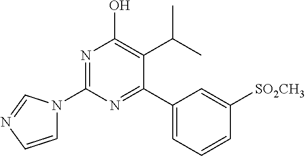 [SMILES: CC(C)c1c(O)nc(-n2ccnc2)nc1-c1cccc(S(C)(=O)=O)c1] |
| 73 | 1 | 2-(1H-Imidazol-1-yl)-6-[1-(3- methylbutyl)-1H-pyrazol-4- yl]-5-(propan-2-yl)pyrimidin- 4-ol | 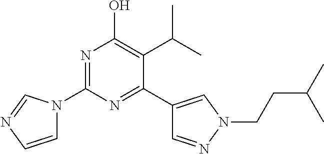 [SMILES: CC(C)CCn1cc(-c2nc(-n3ccnc3)nc(O)c2C(C)C)cn1] |
| 74 | 1 | 2-{4-[6-Hydroxy-2-(1H- imidazol-1-yl)-5-(propan-2- yl)pyrimidin-4-yl]-1H- pyrazol-1-yl}-1-(3-hydroxy- piperidin-1-yl)ethan-1-one | 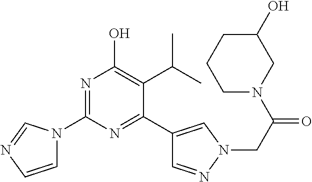 [SMILES: CC(C)c1c(O)nc(-n2ccnc2)nc1-c1cnn(CC(=O)N2CCCC(O)C2)c1] |
| 75 | 1 | 6-(1-tert-Butyl-1H-pyrazol-4- yl)-2-(1H-imidazol-1-yl)-5- (propan-2-yl)pyrimidin-4-ol | 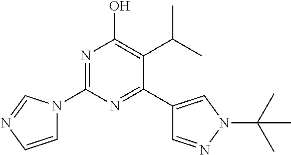 [SMILES: CC(C)c1c(O)nc(-n2ccnc2)nc1-c1cnn(C(C)(C)C)c1] |
| 76 | 1 | N-({3-[6-Hydroxy-2-(1H- imidazol-1-yl)-5-(propan-2- yl)pyrimidin-4-yl]phenyl}- methyl)methanesulfonamide | 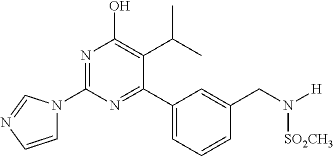 [SMILES: CC(C)c1c(O)nc(-n2ccnc2)nc1-c1cccc(CNS(C)(=O)=O)c1] |
| 77 | 1 | 6-[3-(Hydroxymethyl)- phenyl]-2-(1H-imidazol-1-yl)- 5-(propan-2-yl)pyrimidin-4-ol | 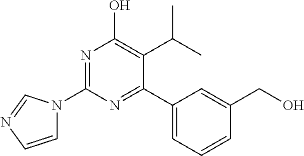 [SMILES: CC(C)c1c(O)nc(-n2ccnc2)nc1-c1cccc(CO)c1] |
| 78 | 1 | 2-(1H-Imidazol-1-yl)-6-(5- methylpyridin-3-yl)-5- (propan-2-yl)pyrimidin-4-ol | 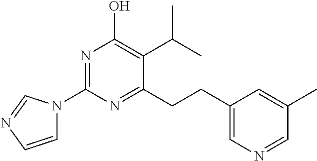 [SMILES: Cc1cncc(CCc2nc(-n3ccnc3)nc(O)c2C(C)C)c1] |
| 79 | 1 | 3-[6-Hydroxy-2-(1H- imidazol-1-yl)-5-(propan-2- yl)pyrimidin-4-yl]benzonitrile | 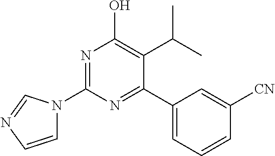 [SMILES: CC(C)c1c(O)nc(-n2ccnc2)nc1-c1cccc(C#N)c1] |
| 80 | 1 | 6-[1-(2-Hydroxyethyl)-1H- pyrazol-4-yl]-2-(1H-imidazol- 1-yl)-5-(propan-2-yl)- pyrimidin-4-ol | 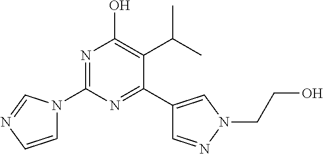 [SMILES: CC(C)c1c(O)nc(-n2ccnc2)nc1-c1cnn(CCO)c1] |
| 81 | 1 | 6-[1-(2-Hydroxy-2-methyl- propyl)-1H-pyrazol-4-yl]-2- (1H-imidazol-1-yl)-5- (propan-2-yl)pyrimidin-4-ol | 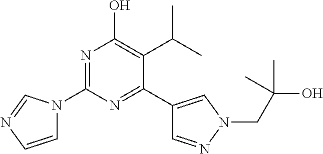 [SMILES: CC(C)c1c(O)nc(-n2ccnc2)nc1-c1cnn(CC(C)(C)O)c1] |
| 82 | 1 | 2-(1H-Imidazol-1-yl)-6-[3- (morpholine-4-carbonyl)- phenyl]-5-(propan-2-yl)- pyrimidin-4-ol | 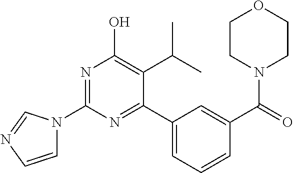 [SMILES: CC(C)c1c(O)nc(-n2ccnc2)nc1-c1cccc(C(=O)N2CCOCC2)c1] |
| 83 | 1 | 2-(1H-Imidazol-1-yl)-6- phenyl-5-(propan-2-yl)- pyrimidin-4-ol | 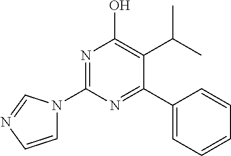 [SMILES: CC(C)c1c(O)nc(-n2ccnc2)nc1-c1ccccc1] |
| 84 | 1 | 6-(Dimethyl-1,2-oxazol-4-yl)- 2-(1H-imidazol-1-yl)-5- (propan-2-yl)pyrimidin-4-ol | 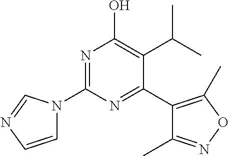 [SMILES: Cc1noc(C)c1-c1nc(-n2ccnc2)nc(O)c1C(C)C] |
| 85 | 1 | 2-({4-[6-Hydroxy-2-(1H- imidazol-1-yl)-5-(propan-2- yl)pyrimidin-4-yl]phenyl}- formamido)acetic acid | 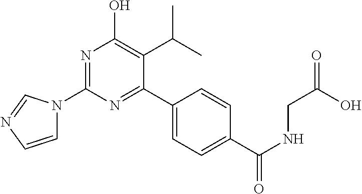 [SMILES: CC(C)c1c(O)nc(-n2ccnc2)nc1-c1ccc(C(=O)NCC(=O)O)cc1] |
| 86 | 1 | 2-(1H-Imidazol-1-yl)-6-[4-(2- methylpropyl)phenyl]-5- (propan-2-yl)pyrimidin-4-ol | 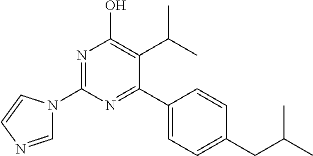 [SMILES: CC(C)Cc1ccc(-c2nc(-n3ccnc3)nc(O)c2C(C)C)cc1] |
| 87 | 1 | Methyl 2-({4-[6-hydroxy-2- (1H-imidazol-1-yl)-5- (propan-2-yl)pyrimidin-4-yl]- phenyl}formamido)acetate | 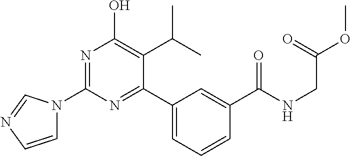 [SMILES: COC(=O)CNC(=O)c1cccc(-c2nc(-n3ccnc3)nc(O)c2C(C)C)c1] |
| 88 | 2 | 6-Chloro-5-(propan-2-yl)-2- [2-(pyrimidin-2-yl)-1H- imidazol-1-yl]pyrimidin-4-ol | 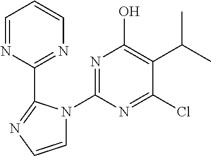 [SMILES: CC(C)c1c(O)nc(-n2ccnc2-c2ncccn2)nc1Cl] |
| 89 | 3 | N-({3-[6-Hydroxy-5-(propan- 2-yl)-2-[2-(pyridin-2-yl)-1H- imidazol-1-yl]pyrimidin-4- yl]phenyl}methyl)methane- sulfonamide | 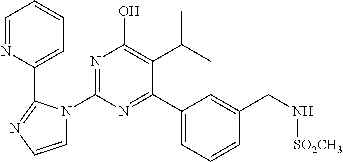 [SMILES: CC(C)c1c(O)nc(-n2ccnc2-c2ccccn2)nc1-c1cccc(CNS(C)(=O)=O)c1] |
| 90 | 6 | N-[3-(Dimethylamino)- propyl]-2-{4-[6-hydroxy-2- (1H-imidazol-1-yl)-5- (propan-2-yl)pyrimidin-4-yl]- 1H-pyrazol-1-yl}acetamide | 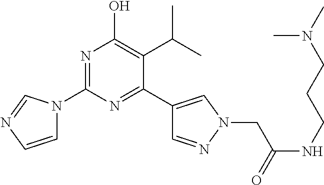 [SMILES: CC(C)c1c(O)nc(-n2ccnc2)nc1-c1cnn(CC(=O)NCCCN(C)C)c1] |
| 91 | 6 | N-[2-(Dimethylamino)ethyl]- 2-{4-[6-hydroxy-2-(1H- imidazol-1-yl)-5-(propan-2- yl)pyrimidin-4-yl]-1H- pyrazol-1-yl}acetamide | 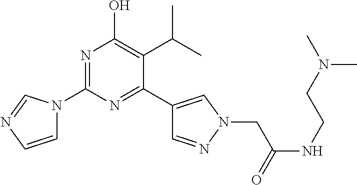 [SMILES: CC(C)c1c(O)nc(-n2ccnc2)nc1-c1cnn(CC(=O)NCCN(C)C)c1] |
| 92 | 6 | 2-{4-[6-Hydroxy-2-(1H- imidazol-1-yl)-5-(propan-2- yl)pyrimidin-4-yl]-1H- pyrazol-1-yl}-N-[2-(4- hydroxypiperidin-1-yl)ethyl]- acetamide | 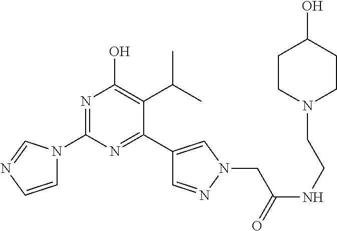 [SMILES: CC(C)c1c(O)nc(-n2ccnc2)nc1-c1cnn(CC(=O)NCCN2CCC(O)CC2)c1] |
| 93 | 6 | N-[4-(Dimethylamino)butyl]- 2-{4-[6-hydroxy-2-(1H- imidazol-1-yl)-5-(propan-2- yl)pyrimidin-4-yl]-1H- pyrazol-1-yl}acetamide | 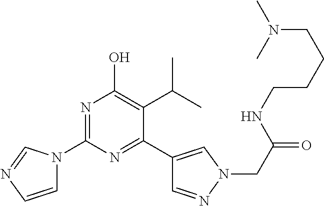 [SMILES: CC(C)c1c(O)nc(-n2ccnc2)nc1-c1cnn(CC(=O)NCCCCN(C)C)c1] |
| 94 | 6 | N-(2-Aminoethyl)-2-{4-[6- hydroxy-2-(1H-imidazol-1- yl)-5-(propan-2-yl)pyrimidin- 4-yl]-1H-pyrazol-1-yl}- acetamide | 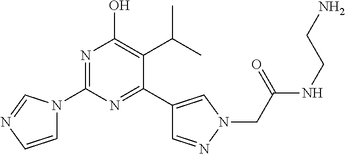 [SMILES: CC(C)c1c(O)nc(-n2ccnc2)nc1-c1cnn(CC(=O)NCCN)c1] |
| 95 | 6 | 2-{4-[6-Hydroxy-2-(1H- imidazol-1-yl)-5-(propan-2- yl)pyrimidin-4-yl]-1H- pyrazol-1-yl}-N-methyl-N-[2- (methylamino)ethyl]- acetamide | 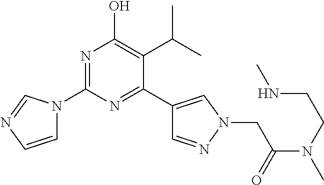 [SMILES: CNCCN(C)C(=O)Cn1cc(-c2nc(-n3ccnc3)nc(O)c2C(C)C)cn1] |
| 96 | 6 | N-[2-(4,4-Difluoropiperidin- 1-yl)ethyl]-2-{4-[6-hydroxy- 2-(1H-Imidazol-1-yl)-5- (propan-2-yl)pyrimidin-4-yl]- 1H-pyrazol-1-yl}acetamide | 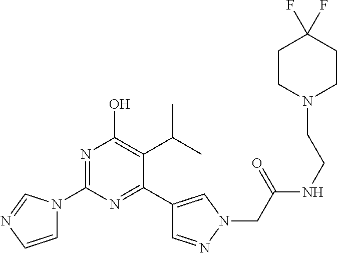 [SMILES: CC(C)c1c(O)nc(-n2ccnc2)nc1-c1cnn(CC(=O)NCCN2CCC(F)(F)CC2)c1] |
| 97 | 6 | 2-{4-[6-Hydroxy-2-(1H- imidazol-1-yl)-5-(propan-2- yl)pyrimidin-4-yl]-1H- pyrazol-1-yl}-N-[(4-methyl- morpholin-2-yl)methyl]- acetamide | 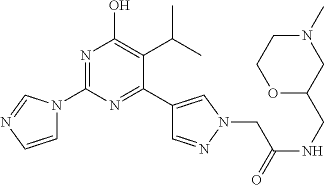 [SMILES: CC(C)c1c(O)nc(-n2ccnc2)nc1-c1cnn(CC(=O)NCC2CN(C)CCO2)c1] |
| 98 | 6 | 1-(3-Aminopiperidin-1-yl)-2- {4-[6-hydroxy-2-(1H- imidazol-1-yl)-5-(propan-2- yl)pyrimidin-4-yl]-1H- pyrazol-1-yl}ethan-1-one | 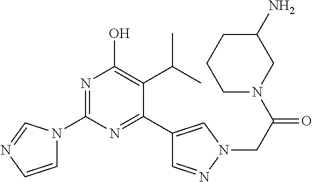 [SMILES: CC(C)c1c(O)nc(-n2ccnc2)nc1-c1cnn(CC(=O)N2CCCC(N)C2)c1] |
| 99 | 6 | 2-{4-[6-Hydroxy-2-(1H- imidazol-1-yl)-5-(propan-2- yl)pyrimidin-4-yl]-1H- pyrazol-1-yl}-1-(4-methyl- piperazin-1-yl)ethan-1-one | 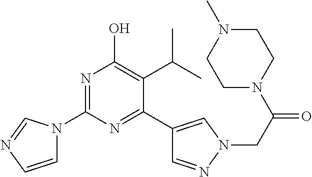 [SMILES: CC(C)c1c(O)nc(-n2ccnc2)nc1-c1cnn(CC(=O)N2CCN(C)CC2)c1] |
| 100 | 6 | 2-{4-[6-Hydroxy-2-(1H- imidazol-1-yl)-5-(propan-2- yl)pyrimidin-4-yl]-1H- pyrazol-1-yl}-N-[2- (morpholin-4-yl)ethyl]- acetamide | 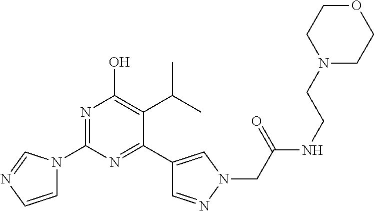 [SMILES: CC(C)c1c(O)nc(-n2ccnc2)nc1-c1cnn(CC(=O)NCCN2CCOCC2)c1] |
| 101 | 6 | 1-(2-{4-[6-Hydroxy-2-(1H- imidazol-1-yl)-5-(propan-2- yl)pyrimidin-4-yl]-1H- pyrazol-1-yl}acetyl)- pyrrolidine-3-carbonitrile | 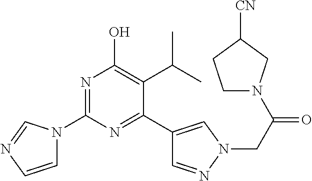 [SMILES: CC(C)c1c(O)nc(-n2ccnc2)nc1-c1cnn(CC(=O)N2CCC(C#N)C2)c1] |
| 102 | 6 | 2-{4-[6-Hydroxy-2-(1H- imidazol-1-yl)-5-(propan-2- yl)pyrimidin-4-yl]-1H- pyrazol-1-yl}-N-{2-[methyl- (phenyl)amino]ethyl}- acetamide | 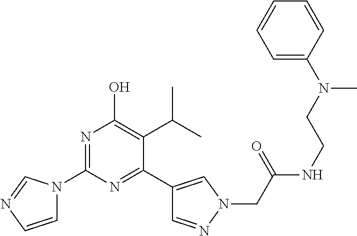 [SMILES: CC(C)c1c(O)nc(-n2ccnc2)nc1-c1cnn(CC(=O)NCCN(C)c2ccccc2)c1] |
| 103 | 6 | 1-(2-{4-[6-Hydroxy-2-(1H- imidazol-1-yl)-5-(propan-2- yl)pyrimidin-4-yl]-1H- pyrazol-1-yl}acetyl)- piperidine-3-carbonitrile | 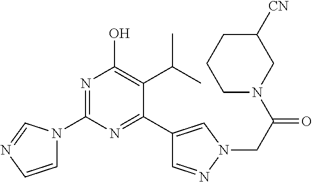 [SMILES: CC(C)c1c(O)nc(-n2ccnc2)nc1-c1cnn(CC(=O)N2CCCC(C#N)C2)c1] |
| 104 | 6 | 2-{4-[6-Hydroxy-2-(1H- imidazol-1-yl)-5-(propan-2- yl)pyrimidin-4-yl]-1H- pyrazol-1-yl}-N-(2-hydroxy- ethyl)acetamide | 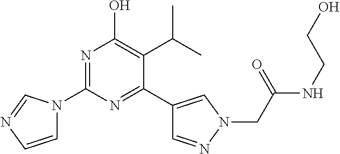 [SMILES: CC(C)c1c(O)nc(-n2ccnc2)nc1-c1cnn(CC(=O)NCCO)c1] |
| 105 | 6 | 2-{4-[6-Hydroxy-2-(1H- imidazol-1-yl)-5-(propan-2- yl)pyrimidin-4-yl]-1H- pyrazol-1-yl}-N-[2-(pyridin- 2-yl)ethyl]acetamide | 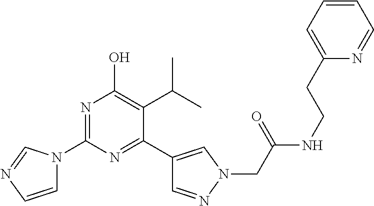 [SMILES: CC(C)c1c(O)nc(-n2ccnc2)nc1-c1cnn(CC(=O)NCCc2ccccn2)c1] |
| 106 | 6 | 2-{4-[6-Hydroxy-2-(1H- imidazol-1-yl)-5-(propan-2- yl)pyrimidin-4-yl]-1H- pyrazol-1-yl}-N-[(1-methyl- 1H-pyrazol-3-yl)methyl]- acetamide | 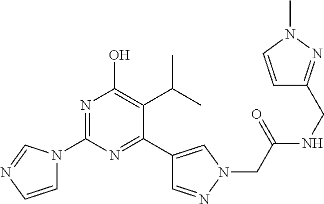 [SMILES: CC(C)c1c(O)nc(-n2ccnc2)nc1-c1cnn(CC(=O)NCc2ccn(C)n2)c1] |
| 107 | 6 | 2-{4-[6-Hydroxy-2-(1H- imidazol-1-yl)-5-(propan-2- yl)pyrimidin-4-yl]-1H- pyrazol-1-yl}-N-(2-methane- sulfonylethyl)acetamide | 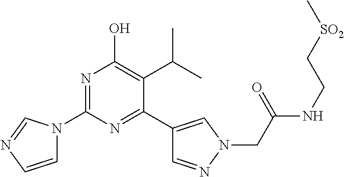 [SMILES: CC(C)c1c(O)nc(-n2ccnc2)nc1-c1cnn(CC(=O)NCCS(C)(=O)=O)c1] |
| 108 | 6 | 2-{4-[6-Hydroxy-2-(1H- imidazol-1-yl)-5-(propan-2- yl)pyrimidin-4-yl]-1H- pyrazol-1-yl}-N-[2-(1H- pyrazol-1-yl)ethyl]acetamide | 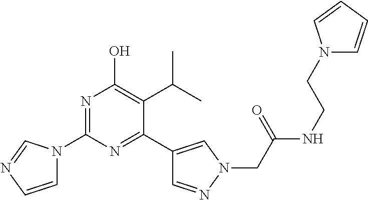 [SMILES: CC(C)c1c(O)nc(-n2ccnc2)nc1-c1cnn(CC(=O)NCCn2cccc2)c1] |
| 109 | 6 | 2-{4-[6-Hydroxy-2-(1H- imidazol-1-yl)-5-(propan-2- yl)pyrimidin-4-yl]-1H- pyrazol-1-yl}-N-[2-(2-oxo- pyrrolidin-1-yl)ethyl]- acetamide | 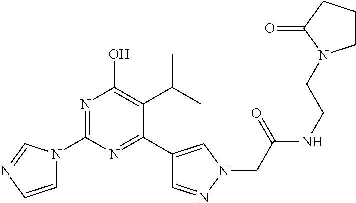 [SMILES: CC(C)c1c(O)nc(-n2ccnc2)nc1-c1cnn(CC(=O)NCCN2CCCC2=O)c1] |
| 110 | 6 | 1-(2-{4-[6-Hydroxy-2-(1H- imidazol-1-yl)-5-(propan-2- yl)pyrimidin-4-yl]-1H- pyrazol-1-yl}acetyl)- piperidine-4-carbonitrile | 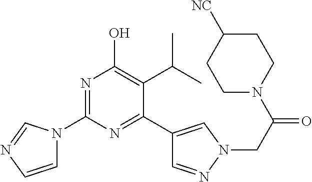 [SMILES: CC(C)c1c(O)nc(-n2ccnc2)nc1-c1cnn(CC(=O)N2CCC(C#N)CC2)c1] |
| 111 | 6 | N-[2-[Cyclopropyl(2,2,2- trifluoroethyl)amino]ethyl}-2- {4-[6-hydroxy-2-(1H- imidazol-1-yl)-5-(propan-2- yl)pyrimidin-4-yl]-1H- pyrazol-1-yl}acetamide | 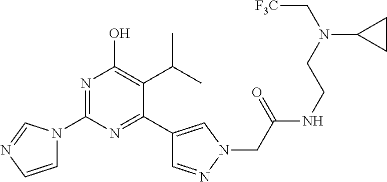 [SMILES: CC(C)c1c(O)nc(-n2ccnc2)nc1-c1cnn(CC(=O)NCCN(CC(F)(F)F)C2CC2)c1] |
| 112 | 6 | 2-{4-[6-Hydroxy-2-(1H- imidazol-1-yl)-5-(propan-2- yl)pyrimidin-4-yl]-1H- pyrazol-1-yl}-1-(3-hydroxy- pyrrolidin-1-yl)ethan-1-one | 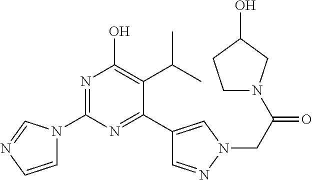 [SMILES: CC(C)c1c(O)nc(-n2ccnc2)nc1-c1cnn(CC(=O)N2CCC(O)C2)c1] |
| 113 | 6 | 2-{4-[6-Hydroxy-2-(1H- imidazol-1-yl)-5-(propan-2- yl)pyrimidin-4-yl]-1H- pyrazol-1-yl)-N-methyl- acetamide | 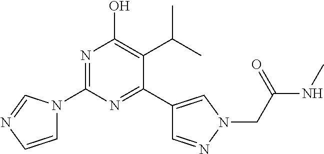 [SMILES: CNC(=O)Cn1cc(-c2nc(-n3ccnc3)nc(O)c2C(C)C)cn1] |
| 114 | 6 | N-(Carbamoylmethyl)-2-{4- [6-hydroxy-2-(1H-imidazol-1- yl)-5-(propan-2-yl)pyrimidin- 4-yl]-1H-pyrazol-1-yl}- acetamide | 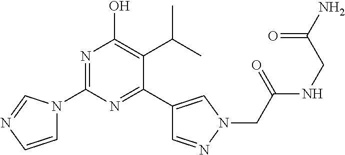 [SMILES: CC(C)c1c(O)nc(-n2ccnc2)nc1-c1cnn(CC(=O)NCC(N)=O)c1] |
| 115 | 6 | 2-{4-[6-Hydroxy-2-(1H- imidazol-1-yl)-5-(propan-2- yl)pyrimidin-4-yl]-1H- pyrazol-1-yl}-N-(1-hydroxy- 3-methylbutan-2-yl)acetamide | 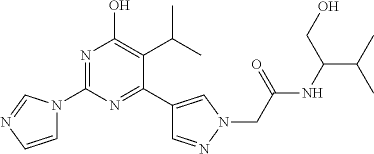 [SMILES: CC(C)c1c(O)nc(-n2ccnc2)nc1-c1cnn(CC(=O)NC(CO)C(C)C)c1] |
| 116 | 6 | 2-{4-[6-Hydroxy-2-(1H- imidazol-1-yl)-5-(propan-2- yl)pyrimidin-4-yl]-1H- pyrazol-1-yl}-N-{2-[methyl- (2,2,2-trifluoroethyl)amino]- ethyl}acetamide | 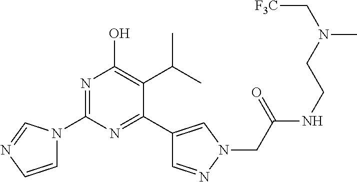 [SMILES: CC(C)c1c(O)nc(-n2ccnc2)nc1-c1cnn(CC(=O)NCCN(C)CC(F)(F)F)c1] |
| 117 | 6 | 2-{4-[6-Hydroxy-2-(1H- imidazol-1-yl)-5-(propan-2- yl)pyrimidin-4-yl]-1H- pyrazol-1-yl}-1-(morpholin- 4-yl)ethan-1-one | 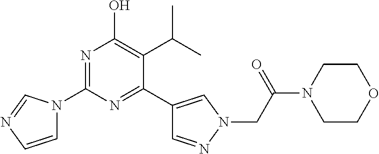 [SMILES: CC(C)c1c(O)nc(-n2ccnc2)nc1-c1cnn(CC(=O)N2CCOCC2)c1] |
| 118 | 6 | 2-{4-[6-Hydroxy-2-(1H- imidazol-1-yl)-5-(propan-2- yl)pyrimidin-4-yl]-1H- pyrazol-1-yl}-N-(2-methoxy- ethyl)acetamide | 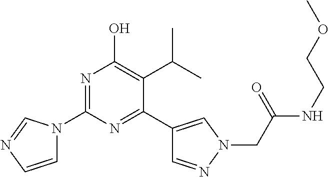 [SMILES: COCCNC(=O)Cn1cc(-c2nc(-n3ccnc3)nc(O)c2C(C)C)cn1] |
| 119 | 6 | 2-{4-[6-Hydroxy-2-(1H- imidazol-1-yl)-5-(propan-2- yl)pyrimidin-4-yl]-1H- pyrazol-1-yl}-1-(4-hydroxy- piperidin-1-yl)ethan-1-one | 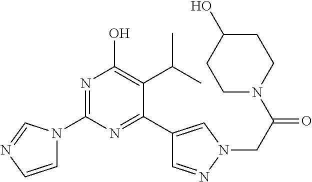 [SMILES: CC(C)c1c(O)nc(-n2ccnc2)nc1-c1cnn(CC(=O)N2CCC(O)CC2)c1] |
| 120 | 8 | 5-[6-Hydroxy-2-(1H- imidazol-1-yl)-5-(propan-2- yl)pyrimidin-4-yl]-N-methyl- pyridine-2-carboxamide | 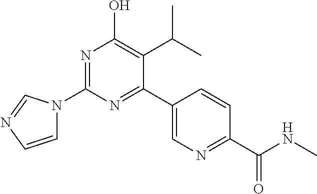 [SMILES: CNC(=O)c1ccc(-c2nc(-n3ccnc3)nc(O)c2C(C)C)cn1] |
| 121 | 8 | 5-[6-Hydroxy-2-(1H- imidazol-1-yl)-5-(propan-2- yl)pyrimidin-4-yl]pyridine-2- carboxylic acid | 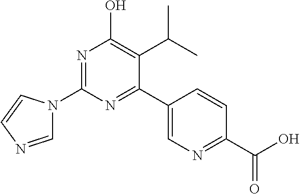 [SMILES: CC(C)c1c(O)nc(-n2ccnc2)nc1-c1ccc(C(=O)O)nc1] |
| 122 | 11 | 2-(1H-Imidazol-1-yl)-6-[2-(4- methylpiperazin-1-yl)ethoxy]- 5-(propan-2-yl)pyrimidin-4-ol | 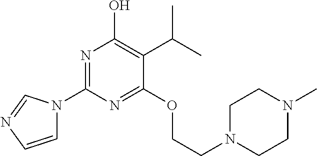 [SMILES: CC(C)c1c(O)nc(-n2ccnc2)nc1OCCN1CCN(C)CC1] |
| 123 | 11 | 2-(1H-Imidazol-1-yl)-6-[2- (morpholin-4-yl)ethoxy]-5- (propan-2-yl)pyrimidin-4-ol | 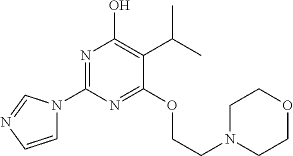 [SMILES: CC(C)c1c(O)nc(-n2ccnc2)nc1OCCN1CCOCC1] |
| 124 | 11 | 2-(1H-Imidazol-1-yl)-6-[2- (piperazin-1-yl)ethoxy]-5- (propan-2-yl)pyrimidin-4-ol | 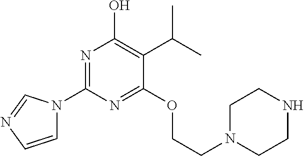 [SMILES: CC(C)c1c(O)nc(-n2ccnc2)nc1OCCN1CCNCC1] |
| 125 | 11 | 1-[4-(2-{[6-Hydroxy-2-(1H- imidazol-1-yl)-5-(propan-2- yl)pyrimidin-4-yl]oxy}ethyl)- piperazin-1-yl]ethan-1-one | 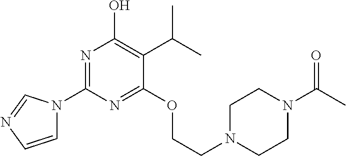 [SMILES: CC(=O)N1CCN(CCOc2nc(-n3ccnc3)nc(O)c2C(C)C)CC1] |
| 126 | 11 | 2-(1H-Imidazol-1-yl)-6- (morpholin-3-ylmethoxy)-5- (propan-2-yl)pyrimidin-4-ol | 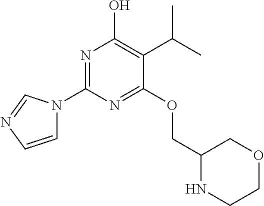 [SMILES: CC(C)c1c(O)nc(-n2ccnc2)nc1OCC1COCCN1] |
| 127 | 11 | Benzyl 3-({[6-hydroxy-2- (1H-imidazol-1-yl)-5- (propan-2-yl)pyrimidin-4-yl]- oxy}methyl)piperazine-1- carboxylate | 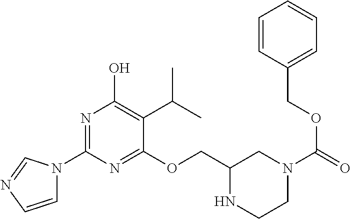 [SMILES: CC(C)c1c(O)nc(-n2ccnc2)nc1OCC1CN(C(=O)OCc2ccccc2)CCN1] |
| 128 | 11 | 6-[(1-Aminocyclopropyl)- methoxy]-2-(1H-imidazol-1- yl)-5-(propan-2-yl)pyrimidin- 4-ol | 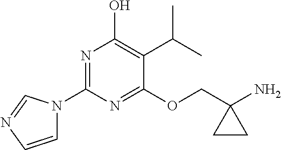 [SMILES: CC(C)c1c(O)nc(-n2ccnc2)nc1OCC1(N)CC1] |
| 129 | 11 | 2-(1H-Imidazol-1-yl)-5- (propan-2-yl)-6-[2-(pyridin-4- yl)ethoxy]pyrimidin-4-ol | 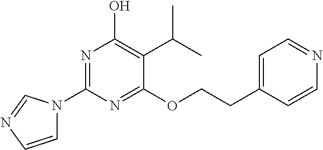 [SMILES: CC(C)c1c(O)nc(-n2ccnc2)nc1OCCc1ccncc1] |
| 130 | 11 | 2-(1H-Imidazol-1-yl)-6-[2- (piperidin-1-yl)ethoxy]-5- (propan-2-yl)pyrimidin-4-ol | 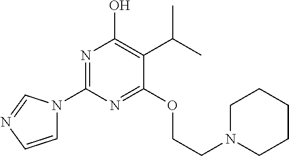 [SMILES: CC(C)c1c(O)nc(-n2ccnc2)nc1OCCN1CCCCC1] |
| 131 | 11 | 2-(1H-Imidazol-1-yl)-6-[2- (oxan-4-yl)ethoxy]-5-(propan- 2-yl)pyrimidin-4-ol | 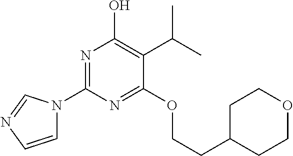 [SMILES: CC(C)c1c(O)nc(-n2ccnc2)nc1OCCC1CCOCC1] |
| 132 | 11 | 1-(2-{[6-Hydroxy-2-(1H- imidazol-1-yl)-5-(propan-2- yl)pyrimidin-4-yl]oxy}ethyl)- pyrrolidin-2-one | 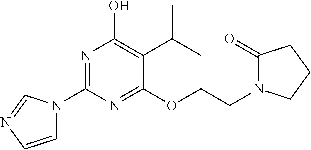 [SMILES: CC(C)c1c(O)nc(-n2ccnc2)nc1OCCN1CCCC1=O] |
| 133 | 11 | Methyl 2-{[6-hydroxy-2-(1H- imidazol-1-yl)-5-(propan-2- yl)pyrimidin-4-yl]oxy}acetate | 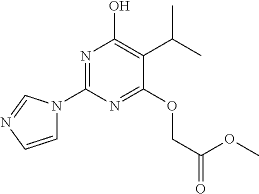 [SMILES: COC(=O)COc1nc(-n2ccnc2)nc(O)c1C(C)C] |
| 134 | 11 | 6-(Benzyloxy)-2-(1H- imidazol-1-yl)-5-(propan-2- yl)pyrimidin-4-ol | 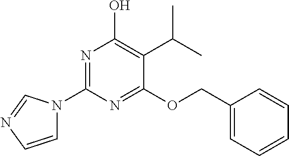 [SMILES: CC(C)c1c(O)nc(-n2ccnc2)nc1OCc1ccccc1] |
| 135 | 11 | 2-(1H-Imidazol-1-yl)-6- (oxolan-3-yloxy)-5-(propan- 2-yl)pyrimidin-4-ol | 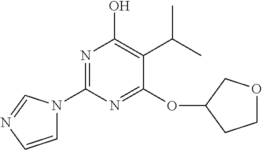 [SMILES: CC(C)c1c(O)nc(-n2ccnc2)nc1OC1CCOC1] |
| 136 | 11 | 2-(1H-Imidazol-1-yl)-5- (propan-2-yl)-6-(pyrimidin-4- ylmethoxy)pyrimidin-4-ol | 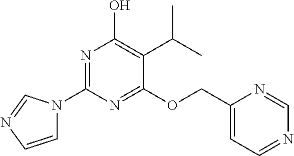 [SMILES: CC(C)c1c(O)nc(-n2ccnc2)nc1OCc1ccncn1] |
| 137 | 11 | 6-[2-(Dimethylamino)- ethoxy]-2-(1H-imidazol-1-yl)- 5-(propan-2-yl)pyrimidin-4-ol | 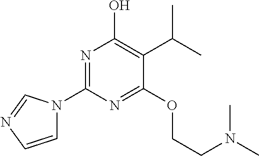 [SMILES: CC(C)c1c(O)nc(-n2ccnc2)nc1OCCN(C)C] |
| 138 | 11 | 2-(1H-Imidazol-1-yl)-6-{[1- (morpholin-4-yl)propan-2- yl]oxy}-5-(propan-2-yl)- pyrimidin-4-ol | 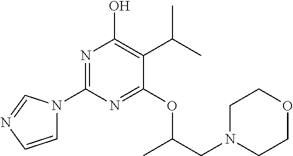 [SMILES: CC(CN1CCOCC1)Oc1nc(-n2ccnc2)nc(O)c1C(C)C] |
| 139 | 11 | 2-(1H-Imidazol-1-yl)-6- (oxan-4-ylmethoxy)-5- (propan-2-yl)pyrimidin-4-ol | 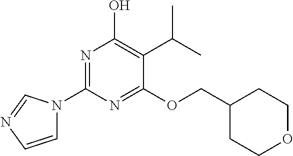 [SMILES: CC(C)c1c(O)nc(-n2ccnc2)nc1OCC1CCOCC1] |
| 140 | 11 | 6-(Hexyloxy)-2-(1H- imidazol-1-yl)-5-(propan-2- yl)pyrimidin-4-ol | 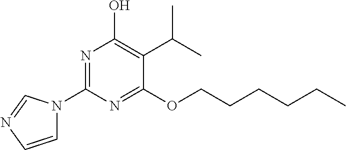 [SMILES: CCCCCCOc1nc(-n2ccnc2)nc(O)c1C(C)C] |
| 141 | 14 | 2-{[6-Hydroxy-2-(1H- imidazol-1-yl)-5-(propan-2- yl)pyrimidin-4-yl]oxy}-N- (pyridin-3-ylmethyl)- acetamide | 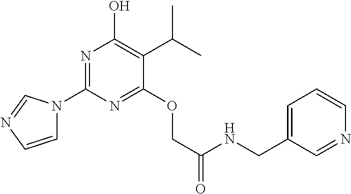 [SMILES: CC(C)c1c(O)nc(-n2ccnc2)nc1OCC(=O)NCc1cccnc1] |
| 142 | 14 | 2-{[6-Hydroxy-2-(1H- imidazol-1-yl)-5-(propan-2- yl)pyrimidin-4-yl]oxy}-1- (morpholin-4-yl)ethan-1-one | 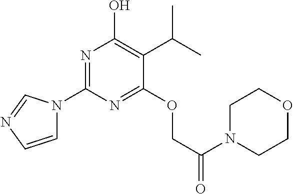 [SMILES: CC(C)c1c(O)nc(-n2ccnc2)nc1OCC(=O)N1CCOCC1] |
| 143 | 19 | 5-(Propan-2-yl)-6-(pyrimidin- 5-ylmethoxy)-2-(1,3-thiazol- 5-yl)pyrimidin-4-ol | 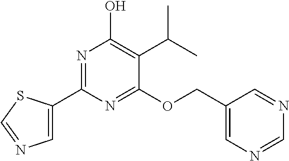 [SMILES: CC(C)c1c(O)nc(-c2cncs2)nc1OCc1cncnc1] |
| 144 | 19 | 5-(Propan-2-yl)-6-(pyridin-4- ylmethoxy)-2-(1,3-thiazol-5- yl)pyrimidin-4-ol | 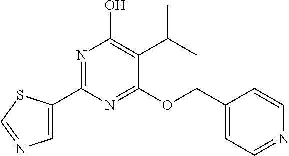 [SMILES: CC(C)c1c(O)nc(-c2cncs2)nc1OCc1ccncc1] |
| 145 | 19 | 5-(Propan-2-yl)-6-(pyridin-2- ylmethoxy)-2-(1,3-thiazol-5- yl)pyrimidin-4-ol | 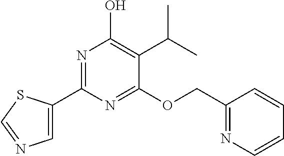 [SMILES: CC(C)c1c(O)nc(-c2cncs2)nc1OCc1ccccn1] |
| 146 | 19 | 6-(2-Aminoethoxy)-5- (propan-2-yl)-2-(1,3-thiazol- 3-yl)pyrimidin-4-ol | 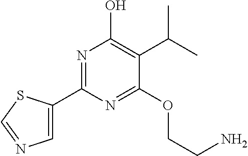 [SMILES: CC(C)c1c(O)nc(-c2cncs2)nc1OCCN] |
| 147 | 19 | 6-(3-Hydroxy-2,2- dimethylpropoxy)-5-(propan- 2-yl)-2-(1,3-thiazol-5-yl)- pyrimidin-4-ol | 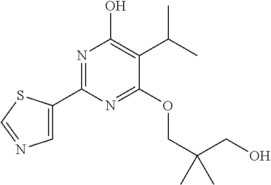 [SMILES: CC(C)c1c(O)nc(-c2cncs2)nc1OCC(C)(C)CO] |
| 148 | 22 | 6-Methyl-2-(2-methyl-1H- imidazol-1-yl)-5-(propan-2- yl)pyrimidin-4-ol | 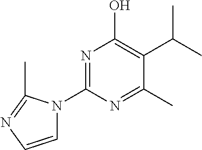 [SMILES: Cc1nc(-n2ccnc2C)nc(O)c1C(C)C] |
| 149 | 26 | 6-Methyl-5-(propan-2-yl)-2- (pyridin-4-yl)pyrimidin-4-ol | 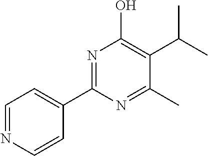 [SMILES: Cc1nc(-c2ccncc2)nc(O)c1C(C)C] |
| 150 | 28 | N-benzyl-5-[6-methyl-4-oxo- 5-(propan-2-yl)-1,4- dihydropyrimidin-2-yl]-1,3- thiazole-4-carboxamide | 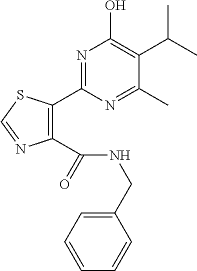 [SMILES: Cc1nc(-c2scnc2C(=O)NCc2ccccc2)nc(O)c1C(C)C] |
| 151 | 29 | 2-(4-Methyl-1H-imidazol-1- yl)-5-(propan-2-yl)-6- (pyridin-3-ylmethoxy)- pyrimidin-4-ol | 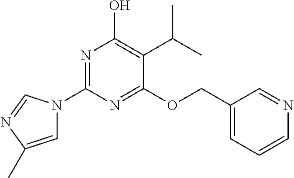 [SMILES: Cc1cn(-c2nc(O)c(C(C)C)c(OCc3cccnc3)n2)cn1] |
| 152 | 30 | 5-(Propan-2-yl)-6-[1-(propan- 2-yl)-1H-pyrazol-4-yl]-2-(1,3- thiazol-5-yl)pyrimidin-4-ol | 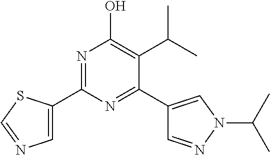 [SMILES: CC(C)c1c(O)nc(-c2cncs2)nc1-c1cnn(C(C)C)c1] |
| 153 | 30 | 6-(1-Methyl-1H-pyrazol-5- yl)-5-(propan-2-yl)-2-(1,3- thiazol-5-yl)pyrimidin-4-ol | 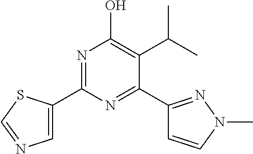 [SMILES: CC(C)c1c(O)nc(-c2cncs2)nc1-c1ccn(C)n1] |
| 154 | 30 | 6-[1-(Piperidin-4-yl)-1H- pyrazol-4-yl]-5-(propan-2-yl)- 2-(1,3-thiazol-5-yl)pyrimidin- 4-ol | 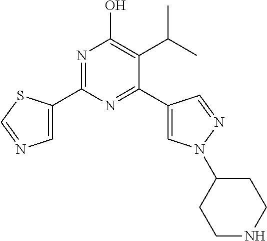 [SMILES: CC(C)c1c(O)nc(-c2cncs2)nc1-c1cnn(C2CCNCC2)c1] |
| 155 | 31 | 6-Methyl-5-(propan-2-yl)-2- (1,3-thiazol-5-yl)-pyrimidin- 4-ol | 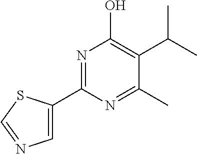 [SMILES: Cc1nc(-c2cncs2)nc(O)c1C(C)C] |
| 156 | 31 | 5-Ethyl-6-methyl-2-(1,3- thiazol-5-yl)-pyrimidin-4-ol | 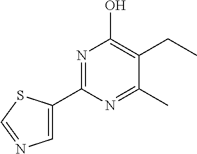 |
| 157 | 31 | 5-(Propan-2-yl)-2-(1,3- thiazol-5-yl)-pyrimidin-4-ol | 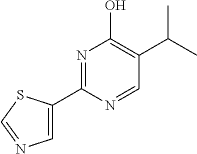 |
| 158 | 31 | 6-Methyl-5-(propan-2-yl)-2- (1,3-thiazol-2-yl)-pyrimidin- 4-ol | 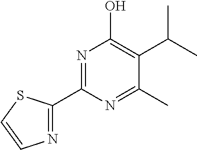 [SMILES: Cc1nc(-c2nccs2)nc(O)c1C(C)C] |

### Biological Activity Assay

### KDM4C and KDM5B AlphaScreen Assay Material and Method

Compounds disclosed herein were (and can be) screened using the following assay.

TABLE 2
Reagents for biological activity assay

| | | |
|---|---|---|
| Biotinylated H3K4Me3 | Anaspec | 64357 |
| (substrate for KDM5B) | | |
| Biotinylated H3K9Me3 | Anaspec | 64360 |
| (substrate for KDM4C) | | |
| Streptavidin donor bead | PerkinElmer | 6760002 |
| Anti-H3K4me1-2 acceptor bead | PerkinElmer | AL116M |
| Anti-H3K9me2 acceptor bead | PerkinElmer | AL117M |
| 1M Hepes | Gibco | 15630-080 |
| 10% Tween-20 | Thermo Scientific | 28320 |
| (NH₄)₂Fe(SO₄)₂ | Fluka | 09719 |
| L-ascorbic acid | Sigma | A5960 |
| Alpha-Ketoglutaric acid | Sigma | K1128 |
| AlphaLisa 5 × Epigenetics Buffer | Perkin Elmer | AL008F |

Peptides used in all experiments were purchased from Anaspe. Full length of KDM5B and KDM4C protein were purchased from Active Motif: KDM5B (cat#31832) and KDM4C (Cat #31858). Final concentration of KDM5B and KDM4C used in the assay were 0.5 nM and 0.2 nM. Final concentration of peptides for 5B and 4C used in the assay were 3 nM.

AlphaScreen assays was conducted as following protocol: buffer used to make compound plate or DMSO is composed of 50 mM HEPES pH 7.4, 0.003% Tween-20; buffer used to dilute enzyme is composed of 50 mM HEPES pH 7.4, 0.003% Tween-20, 0.01% BSA, 500 μM TCEP; and buffer used to dilute the substrate is composed of 50 mM HEPES pH 7.4, 0.003% Tween-20, (NH₄)₂Fe(SO₄)₂ 15 μM, alpha-ketoglutaric acid 4 μM and L-ascorbic acid 100 μM. 4 μL of KDM5B or KDM4C protein were added to 384-well OptiPlate by using multiflow, followed by 4 μl of either buffer or compound. Plates were sealed, spun down and incubated at RT for 15 min. 4 μl of biotinylated peptide was added to the plate, the plate was incubated at room temperature for 25 minutes (KDM5B) and 30 minutes (KDM4C) after sealed and spun down. Streptavidin donor beads and acceptor beads were diluted and mixed in the detection buffer with 7.5 mM EDTA, followed by adding 8 μl of acceptor and donor beads mix to the plate, before the plate was sealed and incubated at room temperature for 3 h or overnight at dark. The plate was read on Envision plate reader (PerkinElmer) using Alphascreen protocol. IC₅₀s were calculated in GraphPad Prism 6.

TABLE 3
Biological activity

| | KDM5B | KDM4C |
|---|---|---|
| | (IC₅₀), | (IC₅₀), |
| Ex. | nM | nM |
| 1 | 12 | 1049 |
| 2 | 229 | 3888 |
| 3 | 372 | 9678 |
| 4 | 125 | 4475 |
| 5 | 117 | 7455 |
| 6 | 4 | 214 |
| 7 | 11 | 1149 |
| 8 | 8 | 752 |
| 9 | 13 | 2387 |
| 10a | 11 | 908 |
| 10b | 11 | 1486 |
| 11 | 7 | 1210 |
| 12 | 107 | 1456 |
| 13 | 12 | 1144 |
| 14 | 32 | 2012 |
| 15 | 651 | 10257 |
| 16 | 26 | 5726 |
| 17 | 148 | 9567 |
| 18 | 27 | 2972 |
| 19 | 91 | 1794 |
| 20 | 100 | 11190 |
| 21 | 1931 | 21128 |
| 22 | 16 | 470 |
| 23 | 24 | 775 |
| 24a | 45 | 499 |
| 24b | 1257 | 16817 |
| 25 | 48 | 613 |
| 26 | 1074 | 15798 |
| 27 | | |
| 28a | 32 | 1748 |
| 28b | 19 | 1647 |
| 29 | 9 | 502 |
| 30 | 345 | 15339 |
| 31 | 74 | 5680 |
| 32 | 4420 | 14122 |
| 33 | 2534 | 3639 |
| 41 | 3 | 281 |
| 42 | 4 | 554 |
| 43 | 9 | 1106 |
| 44 | 10 | 815 |
| 45 | 10 | 1143 |
| 46 | 10 | 1131 |
| 47 | 11 | 771 |
| 48 | 13 | 1258 |
| 49 | 13 | 716 |
| 50 | 14 | 2010 |
| 51 | 14 | 1958 |
| 52 | 14 | 840 |
| 53 | 15 | 1737 |
| 54 | 15 | 896 |
| 55 | 15 | 1337 |
| 56 | 15 | 996 |
| 57 | 16 | 2032 |
| 58 | 16 | 1216 |
| 59 | 16 | 746 |
| 60 | 17 | 1661 |
| 61 | 17 | 1482 |
| 62 | 17 | 1627 |
| 63 | 18 | 1539 |
| 64 | 19 | 2007 |
| 65 | 19 | 937 |
| 66 | 19 | 1177 |
| 67 | 19 | 1341 |
| 68 | 21 | 2451 |
| 69 | 22 | 2055 |
| 70 | 22 | 2658 |
| 71 | 24 | 1562 |
| 72 | 24 | 4468 |
| 73 | 25 | 1353 |
| 74 | 25 | 1746 |
| 75 | 27 | 1242 |
| 76 | 29 | 1856 |
| 77 | 38 | 2460 |
| 78 | 39 | 2660 |
| 79 | 40 | 5194 |
| 80 | 51 | 3486 |
| 81 | 60 | 4042 |
| 82 | 73 | 7969 |
| 83 | 89 | 2693 |
| 84 | 190 | 10717 |
| 85 | 240 | 9710 |
| 86 | 1132 | 10000 |
| 87 | | |
| 88 | 563 | 8263 |
| 89 | 266 | 20000 |
| 90 | 4 | 410 |
| 91 | 5 | 634 |
| 92 | 6 | 525 |
| 93 | 6 | 477 |
| 94 | 6 | 340 |
| 95 | 6 | 278 |
| 96 | 7 | 779 |
| 97 | 7 | 613 |
| 98 | 8 | 505 |
| 99 | 10 | 455 |
| 100 | 12 | 1261 |
| 101 | 12 | 881 |
| 102 | 14 | 1399 |
| 103 | 15 | 909 |
| 104 | 15 | 1378 |
| 105 | 17 | 1077 |
| 106 | 18 | 869 |
| 107 | 19 | 1223 |
| 108 | 19 | 2056 |
| 109 | 20 | 1540 |
| 110 | 21 | 1251 |
| 111 | 21 | 2123 |
| 112 | 21 | 1599 |
| 113 | 24 | 2093 |
| 114 | 26 | 2026 |
| 115 | 30 | 1250 |
| 116 | 30 | 2250 |
| 117 | 31 | 1971 |
| 118 | 33 | 3077 |
| 119 | 39 | 1849 |
| 120 | 18 | 2189 |
| 121 | 18 | 837 |
| 122 | 7 | 1272 |
| 123 | 9 | 1585 |
| 124 | 10 | 1199 |
| 125 | 14 | 2325 |
| 126 | 15 | 840 |
| 127 | 18 | 3077 |
| 128 | 19 | 1399 |
| 129 | 27 | 1117 |
| 130 | 27 | 3675 |
| 131 | 27 | 2091 |
| 132 | 30 | 2415 |
| 133 | 30 | 1328 |
| 134 | 31 | 1449 |
| 135 | 47 | 2053 |
| 136 | 55 | 2409 |
| 137 | 58 | 975 |
| 138 | 65 | 5558 |
| 139 | 75 | 3678 |
| 140 | 341 | 50000 |
| 141 | 33 | 4401 |
| 142 | 35 | 2132 |
| 143 | 138 | 2957 |
| 144 | 140 | 2924 |
| 145 | 163 | 20980 |
| 146 | 1115 | 4245 |
| 147 | 1466 | 12416 |
| 148 | 480 | 9464 |
| 149 | 3319 | 50000 |
| 150 | 1116 | 16856 |
| 151 | 583 | 8785 |
| 152 | 151 | 7239 |
| 153 | 992 | 44733 |
| 154 | 2126 | 13941 |
| 155 | 65 | 1649 |
| 156 | 660 | 8059 |
| 157 | 805 | 5821 |
| 158 | 2408 | 30000 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of formula (I): 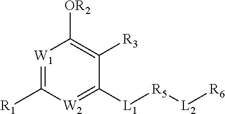
or a salt thereof, wherein:
W₁ and W₂ are independently chosen from N and CH;
R₁ is heteroaryl, which may be optionally substituted with one R₄ group;
R₂ is chosen from H and methyl;
R₃ is chosen from alkyl, cycloalkyl, haloalkyl and halocycloalkyl, any of which may be optionally substituted with one to three R^{z} groups;
R₄ is chosen from alkyl, amino, and heteroaryl, any of which may be optionally substituted with one to three R^{z} groups;
L₁ is chosen from a bond, O, alkyl, alkynyl, alkoxy, amino, alkylamino, amidoalkoxy, acyl, and acylalkoxy;
R₅ is null, halogen, or is chosen from alkyl, cycloalkyl, heterocycloalkyl, aryl, and heteroaryl, any of which may be optionally substituted with one to three R^{x} groups;
L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;
R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{x} groups;
each R^{x} is independently chosen from hydroxy, alkyl, alkoxy, halo, oxo, perhalomethyl, perhalomethoxy, cyano, and NH₂;
each R^{z} is independently chosen from hydroxy, alkyl, alkoxy, halo, oxo, perhalomethyl, perhalomethoxy, cyano, and NH₂.

2. The compound as related in claim 1, wherein R₃ is chosen from C₁₋₄alkyl and C₁₋₄haloalkyl.

3. The compound as related in claim 2, wherein R₃ is C₁₋₄alkyl.

4. The compound as related in claim 3, wherein R₃ isopropyl.

5. The compound as related in claim 1, wherein R₁ is heteroaryl.

6. The compound as related in claim 5, wherein R₁ is chosen from:

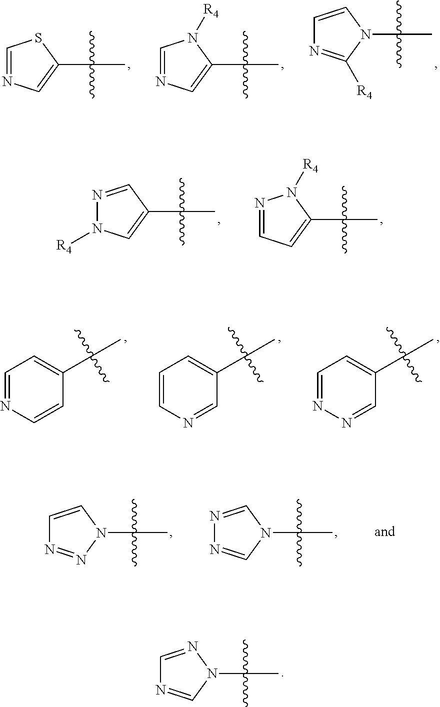

7. The compound as related in claim 5, wherein R₁ is chosen from:

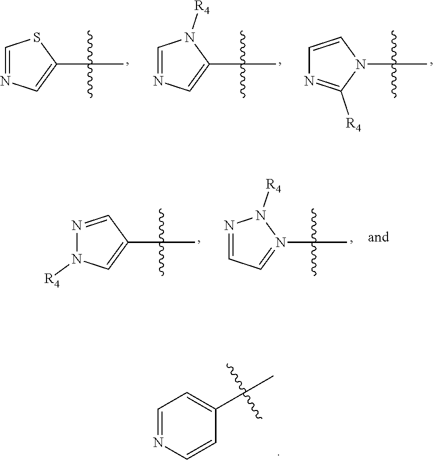

8. The compound as related in claim 1, wherein R₅ is chosen from heterocycloalkyl, aryl, and heteroaryl, any of which may be optionally substituted with one to three R^{z} groups.

9. The compound as related in claim 1, wherein R₅ is chosen from phenyl, imidazolyl, pyrazolyl, pyridinyl, pyrimidinyl, furyl, tetrahydropyranyl, morpholinyl, pyrrolidinonyl, piperidinyl, and piperazinyl, any of which may be optionally substituted with one to three R^{z} groups.

10. The compound as related in claim 1, wherein L₁ is chosen from a bond, O, C₁₋₄alkyl, C₁₋₄alkynyl, C₁₋₄alkoxy, amido, amidoC₁₋₄alkoxy, C₁₋₄alkylamidoC₁₋₄alkoxy, and acylC₁₋₄alkoxy.

11. The compound as related in claim 1, wherein L₂ is chosen from a bond, O, C₁₋₄alkyl, C₁₋₄alkoxy, C₁₋₄alkylamino, aminoC₁₋₄aklylamidoC₁₋₄alkyl, amido, C₁₋₄alkylamido, amidoC₁₋₄alkyl, C₁₋₄alkylamidoC₁₋₄alkyl, amidoC₁₋₄alkoxy, C₁₋₄alkylamidoC₁₋₄alkoxy, acyl, and acylC₁₋₄alkoxy.

12. The compound as related in claim 1, wherein the compound has formula (II): 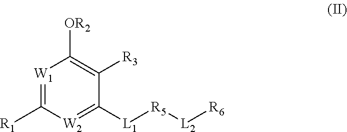
or a salt thereof, wherein:
W₁ and W₂ are independently chosen from N and CH;
R₁ is chosen from 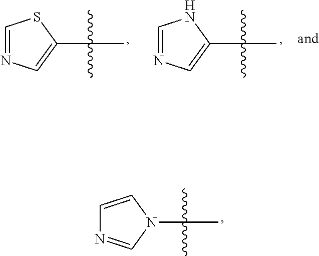 [SMILES: *c1cncs1 ; *c1cnc[nH]1 ; *n1ccnc1]
any of which may be optionally substituted with one R₄ group;
R₂ is chosen from H and methyl;
R₃ is chosen from isopropyl, cyclopropyl, trifluoromethyl and ethyl;
R₄ is chosen from alkyl, amino, and heteroaryl, any of which may be optionally substituted with one to three R^{x} groups;
L₁ is chosen from a bond, O, alkyl, alkynyl, alkoxy, amino, alkylamino, amidoalkoxy, acyl, and acylalkoxy;
R₅ is null, halogen, or is chosen from alkyl, cycloalkyl, heterocycloalkyl, aryl, and heteroaryl, any of which may be optionally substituted with one to three R^{y} groups;
L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;
R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and
each R^{x}, R^{y}, and R^{z} is independently chosen from hydroxy, alkyl, alkoxy, halo, oxo, perhalomethyl, perhalomethoxy, cyano, and NH₂.

13. The compound as related in claim 1, wherein the compound has formula III: 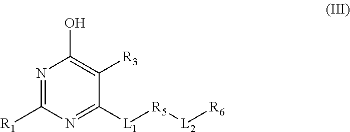
or a salt thereof, wherein:
R₁ is chosen from 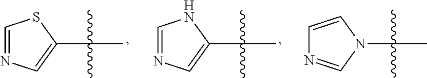 [SMILES: *c1cncs1 ; *c1cnc[nH]1 ; *n1ccnc1]
any of which may be optionally substituted with one R₄ group;
R₃ is chosen from isopropyl, cyclopropyl, trifluoromethyl and ethyl;
R₄ is chosen from alkyl, amino, and heteroaryl, any of which may be optionally substituted with one to three R^{x} groups;
L₁ is chosen from a bond, O, alkyl, alkynyl, alkoxy, amino, alkylamino, amidoalkoxy, acyl, and acylalkoxy;
R₅ is null, halogen, or is chosen from alkyl, cycloalkyl, heterocycloalkyl, aryl, and heteroaryl, any of which may be optionally substituted with one to three R^{y} groups;
L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;
R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and
each R^{x}, R^{y}, and R^{z} is independently chosen from hydroxy, alkyl, alkoxy, halo, oxo, perhalomethyl, perhalomethoxy, cyano, and NH₂.

14. The compound as related in claim 1, wherein the compound has formula IV: 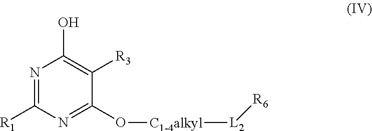
or a salt thereof, wherein:
R₁ is chosen from 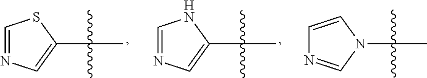 [SMILES: *c1cncs1 ; *c1cnc[nH]1 ; *n1ccnc1]
any of which may be optionally substituted with one R₄ group;
R₃ is chosen from isopropyl, cyclopropyl, trifluoromethyl and ethyl;
R₄ is chosen from alkyl, amino, and heteroaryl, any of which may be optionally substituted with one to three R^{x} groups;
L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;
R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and
each R^{x} and R^{z} is independently chosen from hydroxy, alkyl, alkoxy, halo, oxo, perhalomethyl, perhalomethoxy, cyano, and NH₂.

15. The compound as related in claim 1, wherein the compound has formula V: 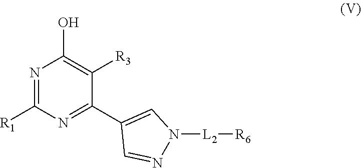
or a salt thereof, wherein:
R₁ is chosen from 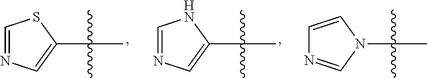 [SMILES: *c1cncs1 ; *c1cnc[nH]1 ; *n1ccnc1]
any of which may be optionally substituted with one R₄ group;
R₃ is chosen from isopropyl, cyclopropyl, trifluoromethyl and ethyl;
R₄ is chosen from alkyl, amino, and heteroaryl, any of which may be optionally substituted with one to three R^{x} groups;
L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;
R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and
each R^{x} and R^{z} is independently chosen from hydroxy, alkyl, alkoxy, halo, oxo, perhalomethyl, perhalomethoxy, cyano, and NH₂.

16. The compound as related in claim 1, wherein the compound has formula VI: 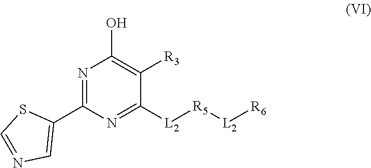
or a salt thereof; wherein:
R₃ is chosen from isopropyl, cyclopropyl, trifluoromethyl and ethyl;
L₁ is chosen from a bond, O, alkyl, alkynyl, alkoxy, amino, alkylamino, amidoalkoxy, acyl, and acylalkoxy;
R₅ is null, halogen, or is chosen from alkyl, cycloalkyl, heterocycloalkyl, aryl, and heteroaryl, any of which may be optionally substituted with one to three R^{y} groups;
L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;
R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and
each R^{y} and R^{z} is independently chosen from hydroxy, alkyl, alkoxy, halo, oxo, perhalomethyl, perhalomethoxy, cyano, and NH₂.

17. The compound as related in claim 1, wherein the compound has formula VII: 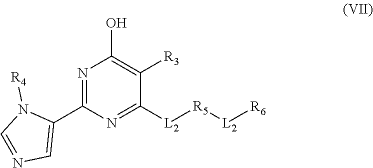
or a salt thereof; wherein
R₃ is chosen from isopropyl, cyclopropyl, trifluoromethyl and ethyl;
R₄ is chosen from alkyl, amino, and heteroaryl, any of which may be optionally substituted with one to three R^{x} groups;
L₁ is chosen from a bond, O, alkyl, alkynyl, alkoxy, amino, alkylamino, amidoalkoxy, acyl, and acylalkoxy;
R₅ is null, halogen, or is chosen from alkyl, cycloalkyl, heterocycloalkyl, aryl, and heteroaryl, any of which may be optionally substituted with one to three R^{y} groups;
L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;
R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and
each R^{x}, R^{y}, and R^{z} is independently chosen from hydroxy, alkyl, alkoxy, halo, oxo, perhalomethyl, perhalomethoxy, cyano, and NH₂.

18. The compound as related in claim 1, wherein the compound has formula VIII: 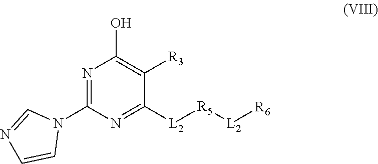
or a salt thereof; wherein:
R₃ is chosen from isopropyl, cyclopropyl, trifluoromethyl and ethyl;
L₁ is chosen from a bond, O, alkyl, alkynyl, alkoxy, amino, alkylamino, amidoalkoxy, acyl, and acylalkoxy;
R₅ is null, halogen, or is chosen from alkyl, cycloalkyl, heterocycloalkyl, aryl, and heteroaryl, any of which may be optionally substituted with one to three R^{y} groups;
L₂ is chosen from null, a bond, O, alkyl, alkoxy, amino, alkylamino, aminoalkyl, amido, alkylamido, amidoalkyl, alkylamidoalkyl, acyl, acylalkoxy, carbonyl, carbonylalkyl, carboxy, alkylcarbonyl and amidoalkylcarboxy;
R₆ is chosen from null, hydrogen, cyano, and hydroxy, or is chosen from alkyl, hydroxy, alkoxy, hydroxyalkyl, amino, aminoalkyl, alkylamino, sulfonylalkyl, sulfonamidoalkyl, carboxyl, cycloalkyl, heterocycloalkyl, aryl, arylalkyl, arylamino, and heteroaryl, any of which may be optionally substituted with one to three R^{z} groups; and
each R^{y} and R^{z} is independently chosen from hydroxy, alkyl, alkoxy, halo, oxo, perhalomethyl, perhalomethoxy, cyano, and NH₂.

19. A compound chosen from Examples 1-158, or a salt thereof.

20. A compound as recited in claim 1 for use as a medicament.

21. A compound as recited in claim 1 for use in the treatment of a hyperproliferative disease.

22. A compound as recited in claim 1 for use in the manufacture of a medicament for the prevention or treatment of a disease or condition ameliorated by the inhibition of KDM5.

23. A pharmaceutical composition comprising a compound as recited in claim 1 together with a pharmaceutically acceptable carrier.

24. A method of inhibition of KDM5 comprising contacting KDM5 with a compound as recited in claim 1.

25. A method of treatment of a KDM5-mediated disease comprising the administration of a therapeutically effective amount of a compound as recited in claim 1 to a patient in need thereof.

26. The method as recited in claim 25 wherein said disease is cancer.

27. A method of treatment of a KDM5-mediated disease comprising the administration of:
a. a therapeutically effective amount of a compound as recited in claim 1; and
b. another therapeutic agent.

28. The method as recited in claim 26 wherein said cancer is chosen from leukemia, lymphoma, oral cancer, laryngeal cancer, esophageal cancer, prostate cancer, bladder cancer, renal cancer, uterine cancer, ovarian cancer, testicular cancer, rectal cancer, colon cancer, lung cancer, brain cancer, breast cancer, pancreatic cancer, stomach cancer, liver cancer, thyroid cancer, melanoma, and multiple myeloma.

29. The method as recited in claim 27 wherein said other agent is chosen from aminoglutethimide, amsacrine, anastrozole, asparaginase, bcg, bicalutamide, bleomycin, buserelin, busulfan, camptothecin, capecitabine, carboplatin, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, irinotecan, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, porfimer, procarbazine, raltitrexed, rituximab, sorafenib, streptozocin, sunitinib, suramin, tamoxifen, temozolomide, temsirolimus, teniposide, testosterone, thioguanine, thiotepa, titanocene dichloride, topotecan, trastuzumab, tretinoin, vinblastine, vincristine, vindesine, and vinorelbine.

30. The method of claim 26, wherein the method further comprises administering non-chemical methods of cancer treatment.

31. The method of claim 30, wherein the method further comprises administering radiation therapy.

32. The method of claim 30, wherein the method further comprises administering surgery, thermoablation, focused ultrasound therapy, cryotherapy, or any combination thereof.

33. A method for achieving an effect in a patient comprising the administration of a therapeutically effective amount of a compound as recited in claim 1 to a patient, wherein the effect is chosen from decreased chemoresistance and decreased likelihood of relapse.
